(19) United States
(12) Patent Application Publication
(10) Pub. No.: US 2025/0057952 A1
(43) Pub. Date: Feb. 20, 2025

(54) ENHANCING EFFICACY OF T-CELL-MEDIATED IMMUNOTHERAPY BY MODULATING CANCER-ASSOCIATED FIBROBLASTS IN SOLID TUMORS

(71) Applicant: CELLECTIS S.A., Paris (FR)

(72) Inventors: Shipra DAS, New York, NY (US); Julien VALTON, Paris (FR); Laurent POIROT, Paris (FR); Philippe DUCHATEAU, Paris (FR)

(21) Appl. No.: 18/562,603

(22) PCT Filed: May 23, 2022
(86) PCT No.: PCT/EP2022/063899

§ 371 Date: Nov. 20, 2023

Related U.S. Application Data
(60) Provisional application No. 63/191,699, filed on May 21, 2021.

(30) Foreign Application Priority Data
Nov. 4, 2021 (DK) PA202170544

(51) Int. Cl. A61K 39/00; A61K 39/395; A61P 35/00; C12N 15/90

(52) U.S. Cl. CPC A61K 39/464458; A61K 39/39558; A61K 39/4611; A61K 39/4621; A61K 39/4631; A61K 39/464468; A61P 35/00; C12N 15/907

(57) ABSTRACT

The invention relates to methods of treatment of a solid tumor in a patient in need thereof, comprising administering to the patient: (i) an effective amount of engineered immune cells originating from a donor expressing at their cell surface a Chimeric Antigen Receptor (CAR) directed against Fibroblast Activation Protein (FAP), and (ii) an effective amount of an immunotherapy treatment that elicits an immune response in the patient.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Feb. 22, 2024, is named 18562603_SL 022224.txt and is 196,199 bytes in size.

### FIELD OF THE INVENTION

The present invention generally relates to the field of cancer, in particular, cell therapies and immunotherapies for the treatment of solid tumors in patients.

### BACKGROUND

Adoptive cell therapy, also known as cellular immunotherapy, is a form of treatment that uses the cells of our immune system to eliminate pathological cells, such as infected or malignant cells. Some of these approaches involve directly isolating our own immune cells and simply expanding their numbers, whereas others involve genetically engineering immune cells from patients (autologous approach) or donors (allogeneic approach) to boost and/or redirect them towards specific target tissues. In the case of cancer, immune cells, especially immune cytolytic lymphocytes, Natural Killers and Antigen Presenting Cells/Macrophages, are particularly powerful against cancer, due to their ability to bind to markers known as antigens on the surface of cancer cells. Cellular immunotherapies take advantage of this natural ability and can be deployed in different ways: Tumor-Infiltrating Lymphocyte (TIL) therapy, Engineered T Cell Receptor (TCR) therapy, Chimeric Antigen Receptor (CAR) T Cell therapy and Natural Killer (NK) Cell therapy.

Chimeric antigen receptors (“CAR”) expressing immune cells are cells which have been genetically engineered to express chimeric antigen receptors (CARs) usually designed to recognize specific tumor antigens and kill cancer cells that express said tumor antigen(s). These are generally T-cells expressing CARs (“CAR-T cells”) or Natural Killer cells expressing CARs (“CAR-NK cells”) or macrophages expressing CARs.

CARs are synthetic receptors consisting of a targeting moiety that is associated with one or more signalling domains in a single or multiple fusion molecule(s). In general, the binding moiety of a CAR consists of an antigen-binding domain of a single-chain antibody (scFv), comprising the light and heavy variable fragments of a monoclonal antibody joined by a flexible linker. Binding moieties based on receptor or ligand domains have also been used successfully. The signalling domains for first generation CARs are derived from the cytoplasmic region of the ζCD3zeta or the Fc receptor gamma chains.

First generation CARs have been shown to successfully redirect T-cell cytotoxicity, however, they failed to provide prolonged expansion and anti-tumor activity in vivo. Signalling domains from co-stimulatory molecules including CD28, OX-40 (CD134), ICOS and 4-1BB (CD137) have been added alone (second generation) or in combination (third generation) to enhance survival and increase proliferation of CAR modified T-cells. CARs have successfully allowed T-cells to be redirected against antigens expressed at the surface of tumor cells from various malignancies including lymphomas and solid tumors (Jena, Dotti et al. 2010, Blood 116 (7): 1035-44).

Adoptive immunotherapy, which involves the transfer of autologous or allogeneic antigen-specific T-cells generated ex vivo, is a promising strategy to treat viral infections and cancer as confirmed by the increase in the number of CAR-T cells clinical trials.

So far, only autologous CAR T-cells have been approved by the US Food and Drug Administration (FDA) (e.g. Novartis' anti-CD19 CAR-T tisagenlecleucel (Kymriah™) for the treatment of precursor B-cell acute lymphoblastic leukemia, Kite Pharma's anti-CD19 CAR-T axicabtagene ciloleucel (Yescarta™) for certain types of large B-cell lymphoma in adult patients expressing CD19 as a marker). Allogeneic approaches are more challenging due to the alloreactivity of the cells with respect to the patient's own immune cells. The most advanced programs consist of inactivating endogenous T-cell receptor genes by using specific rare-cutting endonucleases, in particular TALE-nucleases, to reduce the alloreactivity of the cells prior to administering them to patients as reported by Poirot et al. (Multiplex Genome-Edited T-cell Manufacturing Platform for “Off-the-Shelf” Adoptive T-cell Immunotherapies (2015) Cancer. Res. 75 (18): 3853-3864) and Qasim, W. et al. (Molecular remission of infant B-ALL after infusion of universal TALEN gene-edited CAR-T cells. Science Translational 9 (374)). Meanwhile, inactivation of TCR in primary T-cells can be combined with the inactivation of MHC components such as β2m and also further genes encoding checkpoint inhibitor proteins, such as described for instance in WO 2014/184744.

T-cell mediated anti-tumor cytotoxicity is a promising immunotherapeutic strategy for both leukemia and solid tumors. Prominent among these are checkpoint inhibitors (PD-1/PD-L1 inhibitors, CTLA4 inhibitors) as well as tumor-antigen targeted CAR-T therapy. However, several factors limit the efficacy of these strategies against solid tumors, including lack of tumor-infiltrating lymphocytes (TIL) and an immune suppressive tumor microenvironment (TME). Stern et al., Cancer Treat Res. (2020); 180:297-326. Most solid tumor microenvironments are characterized by the presence of activated fibroblasts called cancer-associated fibroblasts (CAFs) that express unique surface proteins such as FAP (Kalluri R., Nat Rev Cancer. (2016); 16:582-98). CAFs can inhibit TILs and promote immune suppression (Wang et al., Cancer Immunol Res. (2014); 2:154-66).

More recently, Choi et al. (CAR-T cells secreting BiTEs circumvent antigen escape without detectable toxicity (2019) Nature Biotech 37:1049-58) have engineered autologous CAR-T cells to circumvent antigen escape by the expression of bi-specific T-cells engagers (BiTE). These transgenic BiTEs, which are secreted by autologous CAR-T cells bind, on the one hand, to target antigens CD19 or EGFRvIII, and on the other hand, to TCR by targeting CD3 antigen. These BiTEs help bringing together a patient's autologous T-cells with the tumor cells that are either CD19 or EGFRvIII positive, thereby optimizing CAR-T efficiency and limiting antigen escape. However, this approach could not be applied in allogeneic treatment settings where patient's immune cells are generally depleted by a previous lymphodepletion regimen and the allogeneic immune cells are TCR deficient (lack CD3 at the cell surface).

The treatment of cancer, particularly treatment of cancers characterized by solid tumors remains a great challenge in healthcare. What is needed are new compositions and treatments that are effective against solid tumors in patients. More particularly needed are new “universal” compositions and treatments which are useful in treating solid tumors in all patients without any allogeneic limitations as, generally, the patients are not the donors of the cells from which said compositions have been prepared.

This background information is provided for informational purposes only. No admission is necessarily intended, nor should it be construed, that any of the preceding information constitutes prior art against the present invention.

### SUMMARY OF THE INVENTION

It is to be understood that both the foregoing general description of the embodiments and the following detailed description are exemplary, and thus do not restrict the scope of the embodiments.

The invention is particularly suited for a “universal” treatment of solid cancers, where the components thereof can be used in many unrelated patients.

In a general aspect, the invention provides a method of treating a solid tumor in a patient in need thereof, comprising administering to the patient (i) an effective amount of engineered immune cells originating from a donor, or from a cell line, expressing at their cell surface a Chimeric Antigen Receptor (CAR) directed against Fibroblast Activation Protein (FAP), and (ii) an effective amount of an immunotherapy treatment that elicits an immune response in the patient.

Said engineered immune cells may be T cells or NK cells.

Although the various aspects of the invention applied to the situation where said immune cells are T cells are detailed herewith, these various aspects similarly apply to NK cells and are, thus, included in the present application.

In a particular aspect, the invention provides a method of treating a solid tumor in a patient in need thereof, comprising administering to the patient (i) an effective amount of engineered T-cells comprising an inactivated TCR, or engineered NK cells, expressing at their cell surface a Chimeric Antigen Receptor (CAR) directed against Fibroblast Activation Protein (FAP), and (ii) an effective amount of an immunotherapy treatment that elicits an immune response in the patient.

In another aspect, the invention provides an engineered T-cell expressing at its cell surface a Chimeric Antigen Receptor (CAR) directed against Fibroblast Activation Protein (FAP),
wherein the CAR comprises:
   (a) an extracellular ligand binding-domain comprising VH and VL amino acid sequences from a monoclonal anti-FAP antibody,
   (b) a hinge amino acid sequence selected from a FcγRIII hinge, a CD8α hinge and an IgG1 hinge,
   (c) a transmembrane domain amino acid sequence comprising a CD8α transmembrane domain or a CD28 transmembrane domain, and
   (d) a cytoplasmic domain comprising amino acid sequences from a CD3 zeta signaling domain and a co-stimulatory domain from 4-1BB or from CD28; and
   wherein the T-cell has been genetically modified to suppress or repress expression of T-cell receptor (TCR) by inactivation of TCR and, optionally, to suppress or repress expression of at least one MHC protein, preferably β2m or HLA, and, optionally to suppress or repress expression of CD52, in the T-cell.

In another aspect, the invention provides an engineered NK-cell expressing at its cell surface a Chimeric Antigen Receptor (CAR) directed against Fibroblast Activation Protein (FAP),
wherein the CAR comprises:
   (a) an extracellular ligand binding-domain comprising VH and VL amino acid sequences from a monoclonal anti-FAP antibody,
   (b) a hinge amino acid sequence selected from a FcγRIII hinge, a CD8α hinge and an IgG1 hinge,
   (c) a transmembrane domain amino acid sequence comprising a CD8α transmembrane domain or a CD28 transmembrane domain, and
   (d) a cytoplasmic domain comprising amino acid sequences from a CD3 zeta signaling domain and a co-stimulatory domain from 4-1BB or from CD28; and
   wherein, optionally, the NK-cell has been genetically modified to suppress or repress expression of at least one MHC protein, preferably β2m or HLA, and, optionally to suppress or repress expression of CD52, in the NK-cell.

In another aspect, the invention provides a pharmaceutical composition comprising (i) engineered T-cells comprising an inactivated TCR, or engineered NK-cells, expressing at their cell surface a Chimeric Antigen Receptor (CAR) directed against Fibroblast Activation Protein (FAP), and (ii) an immunotherapy treatment for eliciting an immune response in a patient, wherein both components (i) and (ii) are formulated for separate administration.

In another aspect, the invention provides a composition comprising engineered T-cells comprising an inactivated TCR, or engineered NK-cells, expressing at their cell surface a Chimeric Antigen Receptor (CAR) directed against Fibroblast Activation Protein (FAP) for use in the treatment of a solid tumor in a patient in need thereof, wherein said engineered cells are administered in combination with an immunotherapy treatment for eliciting an immune response in said patient.

In another aspect, the invention provides a composition comprising an immunotherapy treatment for eliciting an immune response in a patient for use in the treatment of a solid tumor in said patient, wherein said immunotherapy treatment is administered in combination with engineered T-cells comprising an inactivated TCR, or engineered NK-cells, expressing at their cell surface a Chimeric Antigen Receptor (CAR) directed against Fibroblast Activation Protein (FAP).

In some embodiments, the anti-FAP-CAR will be constitutively expressed in engineered CAR-T or CAR-NK cells either through lentiviral integration or through nuclease-mediated cDNA insertion at active gene loci such as TRAC, β2M, or CD52. Additionally, in some embodiments, the TRAC and B2M gene loci can be disrupted, for instance, by TALE-Nuclease to inhibit graft versus host disease (GvHD) and increase CAR-T cell, or CAR-NK cell, persistence in an allogeneic setting.

In some embodiments, the anti-FAP-CAR treatment can be combined with checkpoint blockade that can be induced by using anti-PD-1 inhibitors, anti-PD-L1 inhibitors, anti-CTLA4 inhibitors, or anti-LAG-3 inhibitors. In some embodiments, the anti-FAP-CAR treatment can be combined with an immunotherapy such as tumor-targeting bispecific T-cell engagers. In some embodiments, said tumor-antigen targeting immune cell engagers can be directed against MUC1, Mesothelin, EGFR, VEGF, or Trop2.

In another aspect, the invention provides a method of producing a population of engineered T-cells, comprising:
(i) providing a population of genetically engineered T-cells originating from a donor, in which expression of a T-cell receptor gene is reduced or suppressed; or providing a population of T-cells originating from a donor and reducing or suppressing expression of a T-cell receptor gene in said T-cells;
(ii) expressing in the population of T-cells at least one exogenous polynucleotide encoding a CAR comprising (a) an extracellular ligand binding-domain comprising a Heavy Variable chain (VH) and a Light Variable chain (VL) from a monoclonal anti-FAP antibody, (b) a hinge selected from a FcγRIII hinge, a CD8α hinge and an IgG1 hinge, (c) a transmembrane domain selected from a CD8α transmembrane domain or a CD28 transmembrane domain, and (d) a cytoplasmic domain including a CD3 zeta signaling domain and a co-stimulatory domain from 4-1BB or from CD28; and
(iii) optionally, isolating the T-cells which TCR expression at their cell surface is reduced or suppressed.

In another aspect, the invention provides a method of producing a population of engineered NK-cells, comprising:
(i) providing a population of NK-cells originating from a donor or from a cell line;
(ii) expressing in the population of NK-cells at least one exogenous polynucleotide encoding a CAR comprising (a) an extracellular ligand binding-domain comprising a Heavy Variable chain (VH) and a Light Variable chain (VL) from a monoclonal anti-FAP antibody, (b) a hinge selected from a FcγRIII hinge, a CD8α hinge and an IgG1 hinge, (c) a transmembrane domain selected from a CD8α transmembrane domain or a CD28 transmembrane domain, and (d) a cytoplasmic domain including a CD3 zeta signaling domain and a co-stimulatory domain from 4-1BB or from CD28.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. A. Schematic representation of turning a cold tumor into a hot tumor according to one aspect of the invention. Eliminating CAFs by anti-FAP-CAR immune cells (such as UCART-FAP cells) from a “cold” tumor allows T cell infiltration turning the tumor “hot” and prone to elimination by T-cells. B. Schematic representation of cell behavior upon treatment: lymphodepletion leads to temporary elimination of endogenous T cells (black curve), injection of anti-FAP CAR immune cells (grey dotted curve) eliminate CAFs (black dotted curve) enhancing Tumor Infiltrating Lymphocyte (TIL) infiltration and activation of T cells in the tumor (grey curve), enhancing efficacy of an immunotherapy treatment (such as anti-PD1 treatment).

FIG. 2. A. Schematic diagram of B2M^{KO}UCART-FAP cells with knockout of TCRα/β and MHCI expression and FAP-CAR expression. B. Flow cytometry analysis of mock transfected T cells and B2M^{KO}UCART-FAP cells. Expression of FAP-CAR, TCRα/β and MHCI surface expression are shown by arrows.

FIG. 3. A. Schematic representation of FAP-CAR targeted at TRAC locus. B. Flow cytometry analysis of mock transfected T cells and UCART-FAP cells obtained in example 2. FAP-CAR and TCRα/β expression are shown by arrows.

FIG. 4. A. Schema for assessing specific cell lysis activity of engineered B2M^{KO}UCART-FAP cells towards CAFs. B. Flow cytometry analysis of CAF viability/mortality post incubation with mock transfected or B2M^{KO}UCART-FAP cells. Percentage of CAF lysis is indicated in boxes C. Graph representing quantitation of CAF survival upon mock or B2M^{KO}UCART-FAP treatment from three different donors at different CAF:T-cell ratio.

FIG. 5. A. Images of HCC70-NL-GFP with CAF cell spheroids treated with mock indicated CART cells. Green (bright) cells are the HCC70-NL-GFP tumor cells. B. Graph representing quantitation of tumor survival after indicated CAR-T cells treatment. The indicated CAR-T cells were generated from two different donors. MESO=B2M^{KO}UCART-MESO, FAP=B2M^{KO}UCART-FAP.

FIG. 6. A. Transduction efficiency of mouse T cells with FAP-CAR. B. Schematic of treatment: 4-T1 mouse breast cancer cells were orthotopically implanted in immune competent BALB/cJ mice at Day 0 (DO). At Day 9 (D9), mice were treated or not (Mock) with mouse CART-FAP or with anti-PD1 (α-mPD-1). At Day 17 (D17), mice were either euthanized for analysis or further treated with an anti-PD1 antibody (α-mPD-1). C. Number of infiltrated CD8+ T cells detected in tumors at D17. D. IFNγ level produced by infiltrated CD8+ T cells in tumors at D17. E. Tumor volume over time in the different cohorts of treated mice.

FIG. 7. Schematic representation of the experimental design for measuring the effect of UCART-FAP on UCART-MESO combined with an anti-PD-1 inhibitor in NSG mice implanted with HCC70-NanoLuc-GFP mixed with human triple-negative breast tumor derived CAF.

FIG. 8. Anti-tumor activity of the combination of UCART-FAP, UCART-MESO and anti-PD-1 inhibitor in tumor-engrafted mice. A. Tumor weight (g), B. Tumor volume (mm³) at different days post tumor engraftment, C. Survival curve.

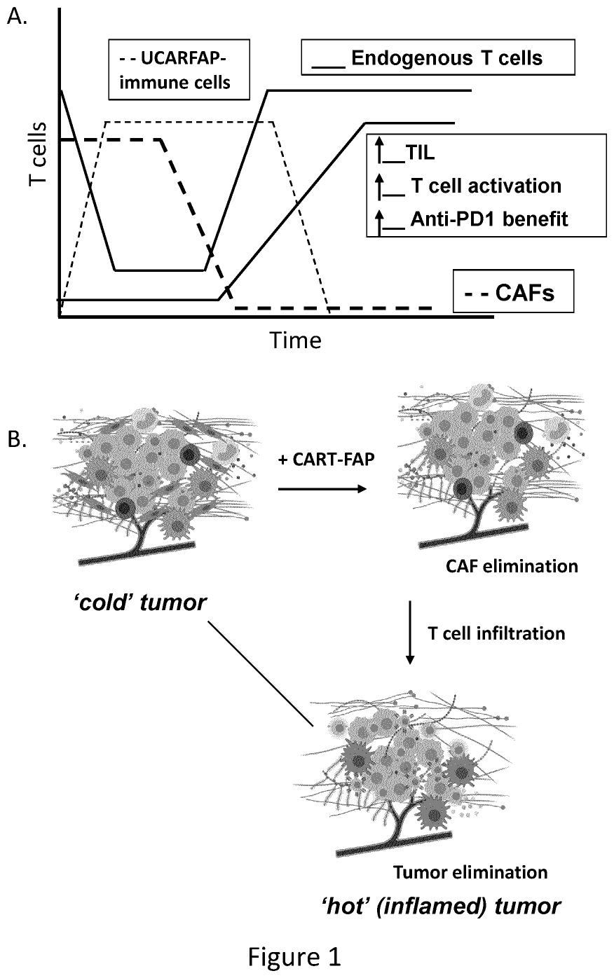

### DETAILED DESCRIPTION

Provided herein are compositions and methods that enable the harnessing of spatial characteristics of the tumor microenvironment (TME) to amplify anti-tumor activity of immunotherapies, such as checkpoint blockade and/or administration of immune cell engagers. More specifically, in some aspects, the invention provides engagement of cancer associated fibroblast (CAF)-targeting anti-FAP CAR as a combination treatment modality to reprogram the solid tumor microenvironment into an inflamed milieu and promote tumor infiltrating lymphocyte (TIL) levels. This TIL rich immune competent microenvironment can then promote efficacy of immunotherapy such as checkpoint blockade and/or immune cell engager therapy.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

Unless specifically defined herein, all technical and scientific terms used have the same meaning as commonly understood by a skilled artisan in the fields of gene therapy, biochemistry, genetics, immunology, cancer and molecular biology. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

As used herein, a “recipient” is a patient that receives a transplant, such as a transplant containing a population of engineered T-cells. The transplanted cells administered to a recipient may be, e.g., autologous, syngeneic, or allogeneic cells.

As used herein, a “donor” is a human or animal from which one or more cells are isolated prior to administration of the cells, or progeny thereof, into a recipient. The one or more cells may be, e.g., a population of immune cells or hematopoietic stem cells to be engineered, expanded, enriched, or maintained according to the methods of the invention prior to administration of the cells or the progeny thereof into a recipient. As contemplated herewith, a “donor” is not the patient to be treated.

“Expansion” in the context of cells refers to increase in the number of a characteristic cell type, or cell types, from an initial cell population of cells, which may or may not be identical. The initial cells used for expansion may not be the same as the cells generated from expansion.

“Cell population” refers to eukaryotic mammalian, preferably human, cells isolated from biological sources, for example, blood product or tissues and derived from more than one cell.

As used herein, the term “pharmaceutical composition” refers to the active agent in combination with a pharmaceutically acceptable carrier and/or excipient e.g. a carrier and/or excipient commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound, cell, or population of cells as disclosed herein into a subject by a method or route which results in at least partial delivery of the agent at a desired site. Pharmaceutical compositions comprising the compounds or cells disclosed herein can be administered by any appropriate route which results in an effective treatment in the subject.

As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

As used herein, the terms “treat,” “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

The term “subject” or “patient” as used herein includes all members of the animal kingdom including non-human primates and humans.

An “effective amount” or “therapeutically effective amount” refers to that amount of a composition described herein which, when administered to a subject (e.g., human), is sufficient to aid in treating a disease. The amount of a composition that constitutes a “therapeutically effective amount” will vary depending on the cell preparations, the condition and its severity, the manner of administration, and the age of the subject to be treated, but can be determined routinely by one of ordinary skill in the art having regard to his own knowledge and to this disclosure. When referring to an individual active ingredient or composition, administered alone, a therapeutically effective dose refers to that ingredient or composition alone. When referring to a combination, a therapeutically effective dose refers to combined amounts of the active ingredients, compositions or both that result in the therapeutic effect, whether administered concurrently, simultaneously, or sequentially.

By “vector” is meant a nucleic acid molecule capable of transporting another nucleic acid to which it has been linked. A “vector” in the present invention includes, but is not limited to, a viral vector, a plasmid, an oligonucleotide, a RNA vector or a linear or circular DNA or RNA molecule which may consists of a chromosomal, non-chromosomal, semisynthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e.g., adenoassociated viruses (AAV), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

As used herein, the term “locus” is the specific physical location of a DNA sequence (e.g. of a gene) into a genome. The term “locus” can refer to the specific physical location of a rare-cutting endonuclease target sequence on a chromosome or on an infection agent's genome sequence. Such a locus can comprise a target sequence that is recognized and/or cleaved by a sequence-specific endonuclease according to the invention. It is understood that the locus of interest of the present invention can not only qualify a nucleic acid sequence that exists in the main body of genetic material (i.e. in a chromosome) of a cell but also a portion of genetic material that can exist independently to said main body of genetic material such as plasmids, episomes, virus, transposons or in organelles such as mitochondria as non-limiting examples.

The term “cleavage” refers to the breakage of the covalent backbone of a polynucleotide. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. Double stranded DNA, RNA, or DNA RNA hybrid cleavage can result in the production of either blunt ends or staggered ends.

“Identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.

“Fibroblast Activation Protein” (“FAP”) is also generally called Prolyl endopeptidase FAP, or Fibroblast Activation Protein alpha.

In one aspect, the invention provides a method of treating a solid tumor in a patient in need thereof, comprising administering to the patient: (i) an effective amount of engineered T-cells, wherein the T-cells comprise an inactivated TCR and express at their cell surface a chimeric antigen receptor (CAR) directed against Fibroblast Activation Protein (FAP), and (ii) an effective amount of an immunotherapy treatment that elicits an immune response in said patient.

In another aspect, the invention provides an engineered T-cell expressing at its cell surface a Chimeric Antigen Receptor (CAR) directed against Fibroblast Activation Protein (FAP), wherein the CAR comprises:
(a) an extracellular ligand binding-domain comprising VH and VL amino acid sequences from a monoclonal anti-FAP antibody,
(b) a hinge amino acid sequence selected from a FcγRIII hinge, a CD8α hinge and an IgG1 hinge,
(c) a transmembrane domain amino acid sequence comprising a CD8α transmembrane domain or a CD28 transmembrane domain, and
(d) a cytoplasmic domain comprising amino acid sequences from a CD3 zeta signaling domain and a co-stimulatory domain from 4-1BB or from CD28; and
   wherein the T-cell has been genetically modified to suppress or repress expression of T-cell receptor (TCR) by inactivation of TCR and, optionally, to suppress or repress expression of at least one MHC protein, preferably β2M or HLA, and optionally to suppress or repress expression of CD52, in the T-cell.

In one embodiment, said engineered T cells comprise either the CD52 or the β2M gene inactivated.

In another aspect, the invention provides a pharmaceutical composition comprising (i) engineered T-cells comprising an inactivated TCR and expressing at their cell surface a Chimeric Antigen Receptor (CAR) directed against Fibroblast Activation Protein (FAP) (UCART-FAP), and (ii) an immunotherapy treatment for eliciting an immune response in a patient, wherein both components (i) and (ii) are formulated for separate administration.

In another aspect, the invention provides a composition comprising engineered T-cells comprising an inactivated TCR and expressing at their cell surface a Chimeric Antigen Receptor (CAR) directed against Fibroblast Activation Protein (FAP) for use in the treatment of a solid tumor in a patient in need thereof, wherein said engineered T-cells are administered in combination with an immunotherapy treatment for eliciting an immune response in said patient.

In another aspect, the invention provides composition comprising an immunotherapy treatment for eliciting an immune response in a patient for use in the treatment of a solid tumor in said patient, wherein said immunotherapy treatment is administered in combination with engineered T-cells comprising an inactivated TCR and expressing at their cell surface a Chimeric Antigen Receptor (CAR) directed against Fibroblast Activation Protein (FAP).

The compositions for use in the immunotherapy treatment and the engineered T-cells can be formulated for separate administration and can be administered concurrently or sequentially. In some embodiments, the composition for use in the immunotherapy treatment is administered after administration of the composition comprising engineered T-cells, for instance the immunotherapy treatment is administered 1 or 2 weeks after administration of the composition comprising the engineered T cells, such as between about 1 or 2 weeks and about 3 to 10 months, between 2 weeks and 8 months, or between 2 weeks and 4 months after administration of the composition comprising the engineered T cells.

In other particular embodiments, the pharmaceutical compositions described herewith further comprise engineered T-cells comprising an inactivated TCR and expressing at their cell surface a Chimeric Antigen Receptor (CAR) directed against an antigen associated with a cancer, preferably a solid tumor antigen, as defined herewith such as Mesothelin, Trop2, MUC1, EGFR, and VEGF. According to these embodiments, this further component is formulated for separate administration from the other two components (i) and (ii).

The engineered cells and methods herein can be part of an autologous or part of an allogenic treatment. By autologous, it is meant that cells used for treating patients are originating from said patient. By allogeneic is meant that the cells or population of cells used for treating patients are not originating from said patient but from a donor or from a cell line.

In some embodiments, the engineered cells are administered to patients undergoing an immunosuppressive treatment. In one embodiment, the administered cells have been made resistant to at least one immunosuppressive agent. In some embodiments, the immunosuppressive treatment helps the selection and expansion of the engineered T-cells within the patient.

The administration of the cells may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions are administered by intravenous injection, where there are capable of migrating to their desired site action.

While individual needs vary, determination of optimal ranges of effective amounts of a given cell type for a particular disease or conditions within the skill of the art. An effective amount means an amount which provides a therapeutic or prophylactic benefit. The dosage administered will be dependent upon the age, health and weight of the recipient, kind of concurrent treatment, if any, frequency of treatment and the nature of the effect desired. In some embodiments, the administration of the cells or population of cells comprises administration of about 10⁴-10⁹ cells per kg body weight. In some embodiments, about 10⁵ to 10⁶ cells/kg body weight are administered. All integer values of cell numbers within those ranges are contemplated.

The cells can be administered in one or more doses. In another embodiment, an effective amount of cells are administered as a single dose. In another embodiment, an effective amount of cells are administered as more than one dose over a period of time. Timing of administration is within the judgment of managing physician and depends on the clinical condition of the patient.

In some embodiments, administering engineered T-cells can include treating the patient with a myeloablative and/or immune suppressive regimen to deplete host bone marrow stem cells and prevent rejection. In some embodiments, the patient is administered chemotherapy and/or radiation therapy. In some embodiments, the patient is administered a reduced dose chemotherapy regimen. In some embodiments, reduced dose chemotherapy regimen with busulfan at 25% of standard dose can be sufficient to achieve significant engraftment of modified cells while reducing conditioning-related toxicity (Aiuti A. et al. (2013), Science 23; 341 (6148)). A stronger chemotherapy regimen can be based on administration of both busulfan and fludarabine as depleting agents for endogenous HSC. In some embodiments, the dose of busulfan and fludarabine are approximately 50% and 30% of the ones employed in standard allogeneic transplantation. In another embodiment, the cells are administered following B-cell ablative therapy such as agents that react with CD20, e.g., Rituxan. In some embodiments, the patient is administered chemotherapy agents such as fludarabine, external-beam radiation therapy (XRT), cyclophosphamide, or antibodies such as OKT3 or CAMPATH.

In certain embodiments, the engineered T-cells are administered to the subject as combination therapy comprising immunosuppressive agents. Exemplary immunosuppressive agents include sirolimus, tacrolimus, cyclosporine, mycophenolate, anti-thymocyte globulin, corticosteroids, calcineurin inhibitor, anti-metabolite, such as methotrexate, post-transplant cyclophosphamide or any combination thereof. In some embodiments, the subject is pretreated with only sirolimus or tacrolimus as prophylaxis against GVHD. In some embodiments, the cells are administered to the subject before an immunosuppressive agent. In some embodiments, the cells are administered to the subject after an immunosuppressive agent. In some embodiments, the cells are administered to the subject concurrently with an immunosuppressive agent. In some embodiments, the cells are administered to the subject without an immunosuppressive agent. In some embodiments, the patient receiving genetically modified cells receives immunosuppressive agent for less than 6 months, 5 months, 4 months, 3 months, 2 months, 1 month, 3 weeks, 2 weeks, or 1 week.

In still further embodiments, the method of treating a solid tumor in a patient in need thereof, comprising administering to the patient (i) an effective amount of engineered TCR-negative immune cells expressing at their cell surface a FAP-CAR, and (ii) an effective amount of an immunotherapy treatment that elicits an immune response in the patient, as described herewith, can further comprise administering (iii) an effective amount of engineered TCR-negative immune cells expressing at their cell surface a CAR binding an antigen associated with a cancer, such as Mesothelin, Trop2, MUC1, EGFR, and VEGF.

In particular further embodiments, the method of treating a solid tumor in a patient in need thereof, comprises administering to the patient (i) an effective amount of engineered TCR-negative immune cells expressing at their cell surface a FAP-CAR, and (ii) an effective amount of an immune checkpoint antagonist that is an antibody directed against an immune checkpoint protein and/or a receptor thereof, wherein the immune checkpoint protein or receptor thereof is selected from the group consisting of PD1, PDL1, CTLA4, LAG3, TIM3, TIGIT, VISTA, GITR and BTLA, and (iii) an effective amount of engineered TCR-negative immune cells expressing at their cell surface a CAR binding an antigen associated with a cancer, such as Mesothelin, Trop2, MUC1, EGFR, and VEGF.

### Engineered Anti-FAP-CAR T-Cells

The engineered T-cells expressing the chimeric antigen receptor directed against Fibroblast Activation Protein (FAP-CAR) are not particularly limiting.

By “chimeric antigen receptor” or “CAR” is generally meant a synthetic receptor comprising a targeting moiety that is associated with one or more signaling domains in a single fusion molecule. As defined herein, the term “chimeric antigen receptor” covers single chain CARs as well as multi-chain CARs. In some embodiments, the binding moiety of a CAR comprises an antigen-binding domain of a single-chain antibody (scFv), comprising light chain and heavy chain variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. First generation CARs have been shown to successfully redirect T-cell cytotoxicity. However, they failed to provide prolonged expansion and anti-tumor activity in vivo. Signaling domains from co-stimulatory molecules including CD28, OX-40 (CD134), and 4-1BB (CD137) have been added alone (second generation) or in combination (third generation) to enhance survival and increase proliferation of CAR modified T-cells. CARs are not necessarily only single chain polypeptides, as multi-chain CARs are also possible. According to the multi-chain CAR architecture, for instance as described in WO 2014/039523, the signalling domains and co-stimulatory domains are located on different polypeptide chains. Such multi-chain CARs can be derived from FcεRI, by replacing the high affinity IgE binding domain of FcεRI alpha chain by an extracellular ligand-binding domain such as scFv, whereas the N- and/or C-termini tails of FcεRI beta and/or gamma chains are fused to signal transducing domains and co-stimulatory domains respectively. The extracellular ligand binding domain has the role of redirecting T-cell specificity towards cell targets, while the signal transducing domains activate the immune cell response.

While the CARs of the present invention and useful in the methods herein are not limited to a specific CAR structure, a nucleic acid that can be used to engineer the immune cells generally encodes a CAR comprising: an extracellular antigen-binding domain that binds to an antigen associated with a disease state (i.e., cancer and the antigen being FAP), a hinge, a transmembrane domain, and an intracellular domain comprising a stimulatory domain and/or a primary signalling domain. Generally, the extracellular antigen-binding domain is a scFv comprising a Heavy variable chain (VH) and a Light variable chain (VL) of an antibody binding to a specific antigen (e.g., to a tumor antigen) connected via a Linker. The transmembrane domain can be, for example, a CD8α transmembrane domain, a CD28 transmembrane domain, or a 4-1BB transmembrane domain. The stimulatory domain can be, for example, the 4-1BB stimulatory domain or CD28 stimulatory domain. The primary signalling domain can be, for example, the CD3 signalling domain.

TABLE 1
Sequence of different domains typically present in a CAR

| Functional | | |
|---|---|---|
| domains | SEQ ID # | amino acid sequence |
| CD8α signal | SEQ ID NO: 114 | MALPVTALLLPLALLLHAARP |
| peptide (or | | |
| sequence leader) | | |
| Alternative signal | SEQ ID NO: 115 | METDTLLLWVLLLWVPGSTG |
| peptide | | |
| FcγRIIIα hinge | SEQ ID NO: 116 | GLAVSTISSFFPPGYQ |
| CD8α hinge | SEQ ID NO: 117 | TTTPAPRPPTPAPTIASQPLSLRPEACRPAAGG |
| | | AVHTRGLDFACD |
| IgG1 hinge | SEQ ID NO: 118 | EPKSPDKTHTCPPCPAPPVAGPSVFLFPPKPKD |
| | | TLMIARTPEVTCVVVDVSHEDPEVKFNWYVDGV |
| | | EVHNAKTKPREEQYNSTYRVVSVLTVLHQDWLN |
| | | GKEYKCKVSNKALPAPIEKTISKAKGQPREPQV |
| | | YTLPPSRDELTKNQVSLTCLVKGFYPSDIAVEW |
| | | ESNGQPENNYKTTPPVLDSDGSFFLYSKLTVDK |
| | | SRWQQGNVFSCSVMHEALHNHYTQKSLSLSPGK |
| CD8α | SEQ ID NO: 119 | IYIWAPLAGTCGVLLLSLVITLYC |
| transmembrane | | |
| domain | | |
| CD28 | SEQ ID NO: 120 | FWVLVVVGGVLACYSLLVTVAFIIFWV |
| transmembrane | | |
| domain | | |
| 4-1BB | SEQ ID NO: 121 | IISFFLALTSTALLFLLFFLTLRFSVV |
| transmembrane | | |
| domain | | |
| 4-1BB co- | SEQ ID NO: 122 | KRGRKKLLYIFKQPFMRPVQTTQEEDGCSCRFP |
| stimulatory | | EEEEGGCEL |
| domain | | |
| CD28 co- | SEQ ID NO: 123 | RSKRSRLLHSDYMNMTPRRPGPTRKHYQPYAP |
| stimulatory | | PRDFAAYRS |
| domain | | |
| CD3ζ signalling | SEQ ID NO: 124 | RVKFSRSADAPAYQQGQNQLYNELNLGRREEY |
| domain | | DVLDKRRGRDPEMGGKPRRKNPQEGLYNELQK |
| | | DKMAEAYSEIGMKGERRRGKGHDGLYQGLST |
| | | ATKDTYDALHMQALPPR |
| Linker 1 | SEQ ID NO: 45 | GSTSGSGKPGSGEGSTK |
| Linker 2 | SEQ ID NO: 46 | GGGGSGGGGSGGGGS |

The CAR comprises amino acid sequences encoding an extracellular ligand (or antigen) binding domain that recognizes FAP. The term “extracellular antigen binding domain” as used herein generally refers to an oligo- or polypeptide that is capable of binding a specific antigen, such as FAP. In some embodiments, the domain will be capable of interacting with a cell surface molecule, such as a ligand. For example, in some embodiments, an extracellular antigen-binding domain may be chosen to recognize an antigen that acts as a cell surface marker on target cells associated with a particular disease state. In a particular instance, said extracellular antigen-binding domain comprises a single chain antibody fragment (scFv) comprising the light (VL) and the heavy (VH) variable fragment of a target-antigen-specific monoclonal antibody joined by a flexible linker. The antigen binding domain of a CAR expressed on the cell surface of the engineered immune cells described herein can be any domain that binds to the target antigen and that derives from, for example, a monoclonal antibody, a recombinant antibody, a human antibody, a humanized antibody, and a functional fragment thereof.

TABLE 2
Sequences of the anti-FAP VH and VL comprised in the ScFv
of preferred anti-FAP CARs

| VH/VL/ | | |
|---|---|---|
| ScFv | CAR | amino acid sequence |
| Heavy | CLSFAP1- | QVQLVQSGAEVKKPGASVKVSCKTSRYTFTEYTIHWVR |
| variable | CAR | QAPGQRLEWIGGINPNNGIPNYNQKFKGRVTITVDTSAS |
| region | | TAYMELSSLRSEDTAVYYCARRRIAYGYDEGHAMDYW |
| | | GQGTLVTVSS (SEQ ID NO: 7) |
| Light | | DIVMTQSPDSLAVSLGERATINCKSSQSLLYSRNQKNYL |
| variable | | AWYQQKPGQPPKLLIFWASTRESGVPDRFSGSGFGTDFT |
| region | | LTISSLQAEDVAVYYCQQYFSYPLTFGQGTKVEI |
| | | (SEQ ID NO: 8) |
| ScFv | | SEQ ID NO: 9 |
| Heavy | CLSFAP2- | EVQLQQSGPELVKPGASVRMSCKASGYTFTDYYMKWV |
| variable | CAR | KQSLGKSLEWIGDIYPNNGEIPYNQKFKGKATLTADKTS |
| region | | STAYMQLNSLTSEDSAVYYCVRGYYYGLAMDYWGQG |
| | | TSVTSVV (SEQ ID NO: 18) |
| Light | | QAVVTQESALTSPGETVTLTCRSSTGAVTTSNYANWVQ |
| variable | | EKPDRLFTGLIGATNNRAPGVPARFSGSLIGDKAALTITG |
| region | | AQTEDEAIYFCALWYSNHFIFGSGTKVTVL |
| | | (SEQ ID NO: 19) |
| ScFv | | SEQ ID NO: 20 |
| Heavy | CLSFAP3- | QVQLVQSGAEVKKPGASVKVSCKASGYTFTENIIHWVR |
| variable | CAR | QAPGQGLEWMGWFHPGSGSIKYNEKFKDRVTMTADTS |
| region | | TSTVYMELSSLRSEDTAVYYCARHGGTGRGAMDYWG |
| | | QGTLVTVSS (SEQ ID NO: 29) |
| Light | | DIQMTQSPSSLSASVGDRVTITCRASKSVSTSAYSYMHW |
| variable | | YQQKPGKAPKLLIYLASNLESGVPSRFSGSGSGTDFTLTI |
| region | | SSLQPEDFATYYCQHSRELPYTFGQGTKLEIKR |
| | | (SEQ ID NO: 30) |
| ScFv | | SEQ ID NO: 31 |
| Heavy | CLSFAP4- | EVQLLESGGGLVQPGGSLRLSCAASGFTFSSYAMSWVR |
| variable | CAR | QAPGKGLEWVSAIIGSGASTYYADSVKGRFTISRDNSKN |
| region | | TLYLQMNSLRAEDTAVYYCAKGWFGGFNYWGQGTLV |
| | | TVSS (SEQ ID NO: 40) |
| Light | | EIVLTQSPGTLSLSPGERATLSCRASQSVTSSYLAWYQQ |
| variable | | KPGQAPRLLINVGSRRATGIPDRFSGSGSGTDFTLTISRL |
| region | | EPEDFAVYYCQQGIMLPPTFGQGTKVEIK (SEQ ID NO: |
| | | 41) |
| ScFv | | SEQ ID NO: 42 |

In some embodiments, the CAR comprises an extracellular binding-domain comprising a VH region comprising SEQ ID NO: 7 and a VL region comprising SEQ ID NO: 8. In some embodiments, the CAR comprises an extracellular binding-domain comprising an amino acid sequence having at least about 80%, 85%, 90%, 91%, 92%, 93%, 94%, 95%, 96%, 97%, 98%, or 99% identity to a VH region comprising SEQ ID NO: 7 and an amino acid sequence having at least about 80%, 85%, 90%, 91%, 92%, 93%, 94%, 95%, 96%, 97%, 98%, or 99% identity to a VL region comprising SEQ ID NO: 8. In some embodiments, the extracellular binding-domain comprises an amino acid sequence comprising complementarity determining regions (CDRs) comprised in SEQ ID NO: 7 and SEQ ID NO: 8. In some embodiments, the H-CDRs comprised in SEQ ID NO: 7 comprise amino acids sequences of SEQ ID NO: 1 to SEQ ID NO: 3. In some embodiments, the L-CDRs comprised in SEQ ID NO: 8 comprise amino acids sequences of SEQ ID NO: 4 to SEQ ID NO: 6. In some embodiments, the CAR comprises an extracellular binding-domain comprising the CDRs comprised in SEQ ID NOs: 7 and 8 and having an amino acid sequence having at least about 80%, 85%, 90%, 91%, 92%, 93%, 94%, 95%, 96%, 97%, 98%, or 99% identity to a VH region comprising SEQ ID NO: 7 and an amino acid sequence having at least about 80%, 85%, 90%, 91%, 92%, 93%, 94%, 95%, 96%, 97%, 98%, or 99% identity to a VL region comprising SEQ ID NO: 8.

In some embodiments, the CAR comprises an extracellular binding-domain comprising a VH region comprising SEQ ID NO: 18 and a VL region comprising SEQ ID NO: 19. In some embodiments, the CAR comprises an extracellular binding-domain comprising an amino acid sequence having at least about 80%, 85%, 90%, 91%, 92%, 93%, 94%, 95%, 96%, 97%, 98%, or 99% identity to a VH region comprising SEQ ID NO: 18 and an amino acid sequence having at least about 80%, 85%, 90%, 91%, 92%, 93%, 94%, 95%, 96%, 97%, 98%, or 99% identity to a VL region comprising SEQ ID NO: 19. In some embodiments, the extracellular binding-domain comprises an amino acid sequence comprising complementarity determining regions (CDRs) comprised in SEQ ID NO: 18 and SEQ ID NO: 19. In some embodiments, the H-CDRs comprised in SEQ ID NO: 18 comprise amino acids sequences of SEQ ID NO: 12 to SEQ ID NO: 14. In some embodiments, the L-CDRs comprised in SEQ ID NO: 19 comprise amino acids sequences of SEQ ID NO: 15 to SEQ ID NO: 17. In some embodiments, the CAR comprises an extracellular binding-domain comprising the CDRs comprised in SEQ ID NOs: 18 and 19 and having an amino acid sequence having at least about 80%, 85%, 90%, 91%, 92%, 93%, 94%, 95%, 96%, 97%, 98%, or 99% identity to a VH region comprising SEQ ID NO: 18, and an amino acid sequence having at least about 80%, 85%, 90%, 91%, 92%, 93%, 94%, 95%, 96%, 97%, 98%, or 99% identity to a VL region comprising SEQ ID NO: 19.

In some embodiments, the CAR comprises an extracellular binding-domain comprising a VH region comprising SEQ ID NO: 29 and a VL comprising SEQ ID NO: 30. In some embodiments, the CAR comprises an extracellular binding-domain comprising an amino acid sequence having at least about 80%, 85%, 90%, 91%, 92%, 93%, 94%, 95%, 96%, 97%, 98%, or 99% identity to a VH region comprising SEQ ID NO: 29 and an amino acid sequence having at least about 80%, 85%, 90%, 91%, 92%, 93%, 94%, 95%, 96%, 97%, 98%, or 99% identity to a VL region comprising SEQ ID NO: 30. In some embodiments, the extracellular binding-domain comprises an amino acid sequence comprising complementarity determining regions (CDRs) comprised in SEQ ID NO: 29 and SEQ ID NO: 30. In some embodiments, the CDRs comprised in SEQ ID NO: 29 comprise amino acids sequences of SEQ ID NO: 23 to SEQ ID NO: 25. In some embodiments, the CDRs comprised in SEQ ID NO: 30 comprise amino acids sequences of SEQ ID NO: 26 to SEQ ID NO: 28. In some embodiments, the CAR comprises an extracellular binding-domain comprising the CDRs comprised in SEQ ID NOs: 29 and 30 and having an amino acid sequence having at least about 80%, 85%, 90%, 91%, 92%, 93%, 94%, 95%, 96%, 97%, 98%, or 99% identity to a VH region comprising SEQ ID NO: 29 and an amino acid sequence having at least about 80%, 85%, 90%, 91%, 92%, 93%, 94%, 95%, 96%, 97%, 98%, or 99% identity to a VL region comprising SEQ ID NO: 30.

In some embodiments, the CAR comprises an extracellular binding-domain comprising a VH region comprising SEQ ID NO: 40 and a VL comprising SEQ ID NO: 41. In some embodiments, the CAR comprises an extracellular binding-domain comprising an amino acid sequence having at least about 80%, 85%, 90%, 91%, 92%, 93%, 94%, 95%, 96%, 97%, 98%, or 99% identity to a VH region comprising SEQ ID NO: 40 and an amino acid sequence having at least about 80%, 85%, 90%, 91%, 92%, 93%, 94%, 95%, 96%, 97%, 98%, or 99% identity to a VL region comprising SEQ ID NO: 41 In some embodiments, the extracellular binding-domain comprises an amino acid sequence comprising complementarity determining regions (CDRs) comprised in SEQ ID NO: 40 and SEQ ID NO: 41. In some embodiments, the CDRs comprised in SEQ ID NO: 40 comprise amino acids sequences of SEQ ID NO: 34 to SEQ ID NO: 36. In some embodiments, the CDRs comprised in SEQ ID NO: 41 comprise amino acids sequences of SEQ ID NO: 37 to SEQ ID NO: 39. In some embodiments, the CAR comprises an extracellular binding-domain comprising the CDRs comprised in SEQ ID NOs: 40 and 41 and having an amino acid sequence having at least about 80%, 85%, 90%, 91%, 92%, 93%, 94%, 95%, 96%, 97%, 98%, or 99% identity to a VH region comprising SEQ ID NO: 40 and an amino acid sequence having at least about 80%, 85%, 90%, 91%, 92%, 93%, 94%, 95%, 96%, 97%, 98%, or 99% identity to a VL region comprising SEQ ID NO: 41.

TABLE 3
Sequences of the CDRs comprised in the scFvs of preferred anti-FAP
CARs

| Chain | CDR1 | CDR2 | CDR3 |
|---|---|---|---|
| CLSFAP | YTFTEYTIH | GINPNNGIPNYNQKF | RRIAYGYDEGHAM |
| 1-heavy | (SEQ ID NO: 1) | (SEQ ID NO: 2) | DY (SEQ ID NO: 3) |
| chain | | | |
| CLSFAP | QSLLYSRNQKNYL | LLIFWASTRES | QQYFSYPLT |
| 1-light | A (SEQ ID NO: 4) | (SEQ ID NO: 5) | (SEQ ID NO: 6) |
| chain | | | |
| CLSFAP | YTFTDYYMK | DIYPNNGEIPYNQKF | VRGYYYGLAMDY |
| 2-heavy | (SEQ ID NO: 12) | (SEQ ID NO: 13) | (SEQ ID NO: 14) |
| chain | | | |
| CLSFAP | TGAVTTSNYAN | GLIGATNNRAP | ALWYSNHFI |
| 2-light | (SEQ ID NO: 15) | (SEQ ID NO: 16) | (SEQ ID NO: 17) |
| chain | | | |
| CLSFAP | YTFTENIIH | WFHPGSGSIKYNEKF | HGGTGRGAMDY |
| 3-heavy | (SEQ ID NO: 23) | (SEQ ID NO: 24) | (SEQ ID NO: 25) |
| chain | | | |
| CLSFAP | KSVSTSAYSYMH | LLIYLASNLES | QHSRELPYT |
| 3-light | (SEQ ID NO: 26) | (SEQ ID NO: 27) | (SEQ ID NO: 28) |
| chain | | | |
| CLSFAP | FTFSSYAMS | VSAIIGSGASTYYAD | KGWFGGFNY |
| 4-heavy | (SEQ ID NO: 34) | SV | (SEQ ID NO: 36) |
| chain | | (SEQ ID NO: 35) | |
| CLSFAP | QSVTSSYLA | LLINVGSRRATGI | QQGIMLPPT |
| 4-light | (SEQ ID NO: 37) | (SEQ ID NO: 38) | (SEQ ID NO: 39) |
| chain | | | |

In some embodiments, the amino acid sequence comprising a VH region and the amino acid sequence comprising a VL region are separated by one or more linker amino acid residues. The number of amino acids constituting the linker is not necessarily limiting, but in some embodiments the linker is at least about 5 amino acids in length, preferably at least about 10 amino acids in length. In some embodiments, the linker is between about 10-25 amino acids in length. In some embodiments, the linker sequence is selected from any one of SEQ ID NOs: 45-46.

In some embodiments, the extracellular ligand binding-domain comprising the VH region and the VL region from a monoclonal anti-FAP antibody comprises a sequence selected from any one of SEQ ID NO: 9, SEQ ID NO: 20, SEQ ID NO: 31 and SEQ ID NO: 42. In some embodiments, the extracellular ligand binding-domain comprises an amino acid sequence having at least about 80%, 85%, 90%, 91%, 92%, 93%, 94%, 95%, 96%, 97%, 98%, or 99% identity to any one of SEQ ID NO: 9, SEQ ID NO: 20, SEQ ID NO: 31 and SEQ ID NO: 42.

In a specific embodiment, the extracellular ligand binding-domain comprising the VH region and the VL region from a monoclonal anti-FAP antibody comprises the amino acid sequence of SEQ ID NO: 9.

In some embodiments, the CAR comprises amino acid sequences encoding an extracellular ligand binding domain that recognizes FAP, a transmembrane domain, and one or more intracellular signalling domains. In some embodiments, the CAR comprises a hinge region that separates the extracellular ligand binding domain and the transmembrane domains.

In some embodiments, the CAR comprises:
(a) an extracellular ligand binding-domain comprising VH and VL from a monoclonal anti-FAP antibody,
(b) a hinge selected from a FcγRIII hinge, a CD8α hinge and an IgG1 hinge,
(c) a transmembrane domain selected from a CD8α transmembrane domain and a CD28 transmembrane domain, and
(d) a cytoplasmic domain including a CD3 zeta signalling domain and a co-stimulatory domain from 4-1BB or CD28.

In some embodiments, the CAR comprises a CD8α hinge.

In some embodiments, said CAR comprises a CD8α hinge, a CD8α transmembrane domain, and a co-stimulatory domain from 4-1BB.

In some embodiments, said CAR comprises a CD8α hinge, a CD28 transmembrane domain, and a co-stimulatory domain from CD28.

In some embodiments, the CAR has an amino acid sequence selected from any one of SEQ ID NO: 10, SEQ ID NO: 11, SEQ ID NO: 21, SEQ ID NO: 22, SEQ ID NO: 32, SEQ ID NO: 33, SEQ ID NO: 43, SEQ ID NO: 44. In some embodiments, the CAR comprises an amino acid sequence having at least about 80%, 85%, 90%, 91%, 92%, 93%, 94%, 95%, 96%, 97%, 98%, or 99% identity to any one of SEQ ID NO: 10, SEQ ID NO: 21, SEQ ID NO: 32, SEQ ID NO: 43.

In some embodiments, the nucleic acid sequence encoding the anti-FAP CAR described herewith comprises a nucleic acid sequence selected from any one of SEQ ID NO: 129, SEQ ID NO: 130, SEQ ID NO: 131, and SEQ ID NO: 132. In some embodiments, the nucleic acid sequence encoding the anti-FAP CAR described herewith comprises a nucleic acid sequence having at least about 80%, 85%, 90%, 91%, 92%, 93%, 94%, 95%, 96%, 97%, 98%, or 99% identity to any one of SEQ ID NO: 129, SEQ ID NO: 130, SEQ ID NO: 131, or SEQ ID NO: 132, and encodes an anti-FAP CAR of amino acid sequence comprising an amino acid sequence selected from any one of SEQ ID NO: 10, SEQ ID NO: 21, SEQ ID NO: 32, and SEQ ID NO: 43, respectively.

In some embodiments, the CAR-T cells according to the present invention for their use in allogeneic settings are endowed with anti-FAP CARs as described herewith comprising a co-stimulatory domain from CD28 in order to trigger a faster T-cells activation. Although such CAR-T-cells usually get more quickly exhausted, it can be advantageous to use CD28 induced CAR-T-cells even if they are not persisting, as the primary goal of the present invention is to make the tumors permeable to the second wave of immunotherapy that is eliciting a specific immune response in the patient.

In some embodiments, the CAR comprises an amino acid sequence having at least about 80%, 85%, 90%, 91%, 92%, 93%, 94%, 95%, 96%, 97%, 98%, or 99% identity to any one of SEQ ID NO: 11, SEQ ID NO: 22, SEQ ID NO: 33, SEQ ID NO: 44.

In some embodiments, the engineered cells according to the invention are made by a process comprising integration, in the genome of said cells, of a lentiviral vector comprising a polynucleotide encoding a FAP-CAR as described herewith.

In some embodiments, the engineered cells expressing the chimeric antigen receptor directed against Fibroblast Activation Protein (FAP-CAR) are made by a process comprising (a) editing at least one gene, by inactivating the gene by inserting into said gene at least one polynucleotide encoding a chimeric antigen receptor specific for FAP (for example, as in any one of the above). In some embodiments, a polynucleotide encoding the CAR is integrated into the endogenous TRAC, B2m, or CD52 locus in the genome of said T-cell.

Therefore, in one embodiment, the invention provides a method of producing a population of engineered T-cells comprising:
(i) providing a population of T-cells originating from a donor;
(ii) inactivating a TCR gene by inserting into the TRAC locus of said T-cells' genome at least one exogenous polynucleotide encoding a CAR comprising: (a) an extracellular ligand binding-domain comprising a Heavy Variable chain (VH) and a Light Variable chain (VL) from a monoclonal anti-FAP antibody, (b) a hinge selected from a FcγRIII hinge, a CD8α hinge and an IgG1 hinge, (c) a CD8α transmembrane domain or a CD28 transmembrane domain, and (d) a cytoplasmic domain including a CD3 zeta signaling domain and a co-stimulatory domain from 4-1BB or from CD28;
(iii) optionally, isolating the T-cells that do not express TCR at their cell surface.

In another embodiment, the T-cell is genetically engineered to have its TCR gene inactivated and the CAR is integrated outside of the TRAC locus in the T-cell's genome.

In one embodiment, the invention provides a method of producing a population of engineered T-cells comprising:
(i) providing a population of T-cells originating from a donor;
(ii) inactivating at least one component of the TCR by site-specific nuclease such as a TALE-nuclease targeting said TCR component;
(iii) expressing in the population of T-cells at least one exogenous polynucleotide encoding a CAR comprising (a) an extracellular ligand binding-domain comprising a Heavy Variable chain (VH) and a Light Variable chain (VL) from a monoclonal anti-FAP antibody, (b) a hinge selected from a FcγRIII hinge, a CD8α hinge and an IgG1 hinge, (c) a CD8α transmembrane domain or a CD28 transmembrane domain, and (d) a cytoplasmic domain including a CD3 zeta signaling domain and a co-stimulatory domain from 4-1BB or from CD28;
(iv) optionally, isolating the T-cells that do not express TCR at their cell surface.

In another embodiment, the invention provides a method of producing a population of engineered T-cells comprising:
(i) providing a population of genetically engineered T-cells originating from a donor, in which expression of a T-cell receptor gene is inactivated;
(ii) expressing in the population of T-cells at least one exogenous polynucleotide encoding a CAR comprising (a) an extracellular ligand binding-domain comprising a Heavy Variable chain (VH) and a Light Variable chain (VL) from a monoclonal anti-FAP antibody, (b) a hinge selected from a FcγRIII hinge, a CD8α hinge and an IgG1 hinge, (c) a CD8α transmembrane domain or a CD28 transmembrane domain, and (d) a cytoplasmic domain including a CD3 zeta signaling domain and a co-stimulatory domain from 4-1BB or from CD28;
(iii) optionally, isolating the T-cells that do not express TCR at their cell surface.

The source for the engineered CAR T-cells is not particularly limiting. T-cells are a type of “immune cells” and by “immune cell” is meant a cell of hematopoietic origin functionally involved in the initiation and/or execution of innate and/or adaptative immune response, such as typically CD45, CD3, CD8 or CD4 positive cells. Immune cells include dendritic cells, killer dendritic cells, mast cells, macrophages, natural killer cells (NK-cell), cytokine-induced killer cells (CIK cells), B-cells or T-cells selected from the group consisting of inflammatory T-lymphocytes, cytotoxic T-lymphocytes, or helper T-lymphocytes, gamma delta T-cells, and Natural killer T-cells (“NKT cell).

In some embodiments, the source of the engineered T-cells are primary cells, and by “primary cell” or “primary cells” are intended cells taken directly from living tissue (e.g. biopsy material) and established for growth in vitro for a limited amount of time, meaning that they can undergo a limited number of population doublings. Primary cells are opposed to continuous tumorigenic or artificially immortalized cell lines. Non-limiting examples of such cell lines are CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; and Molt 4 cells.

Primary immune cells can be obtained from a number of non-limiting sources, including peripheral blood mononuclear cells (PBMC), bone marrow, lymph node tissue, cord blood, thymus tissue, tissue from a site of infection, ascites, pleural effusion, spleen tissue, and from tumors, such as tumor infiltrating lymphocytes. In some embodiments, said immune cell can be derived from a healthy donor, from a patient diagnosed with cancer or from a patient diagnosed with an infection. In another embodiment, said cell is part of a mixed population of immune cells which present different phenotypic characteristics, such as comprising CD4, CD8 and CD56 positive cells. Primary immune cells are provided from donors or patients through a variety of methods known in the art, as for instance by leukapheresis techniques as reviewed by Schwartz J. et al. (Guidelines on the use of therapeutic apheresis in clinical practice-evidence-based approach from the Writing Committee of the American Society for Apheresis: the sixth special issue (2013) J Clin Apher. 28 (3): 145-284).

The immune cells derived from stem cells are also regarded as primary immune cells according to the present invention, in particular those deriving from induced pluripotent stem cells (iPS) (Yamanaka, K. et al. (2008). “Generation of Mouse Induced Pluripotent Stem Cells Without Viral Vectors”. Science. 322 (5903): 949-53). Lentiviral expression of reprogramming factors has been used to induce multipotent cells from human peripheral blood cells (Staerk, J. et al. (2010). “Reprogramming of human peripheral blood cells to induced pluripotent stem cells”. Cell stem cell. 7 (1): 20-4) (Loh, Y H. et al. (2010). “Reprogramming of T cells from human peripheral blood”. Cell stem cell. 7 (1): 15-9).

According to one embodiment of the invention, the immune cells can be derived from human embryonic stem cells by techniques well known in the art that do not involve the destruction of human embryos (Chung et al. (2008) Human Embryonic Stem Cell lines generated without embryo destruction, Cell Stem Cell 2 (2): 113-117).

In some embodiments, the engineered T-cells derive from inflammatory T-lymphocytes, cytotoxic T-lymphocytes, or helper T-lymphocytes.

In some embodiments, the T-cell according to the present invention can be derived from a stem cell. The stem cells can be adult stem cells, embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells.

In another embodiment, the engineered cells can be derived from the group consisting of CD4+T-lymphocytes and CD8+T-lymphocytes. Prior to expansion and genetic modification of the cells of the invention, a source of cells can be obtained from a subject through a variety of non-limiting methods. T-cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T-cell lines available and known to those skilled in the art, may be used. In another embodiment, said cell can be derived from a healthy donor, from a patient diagnosed with cancer or from a patient diagnosed with an infection. In another embodiment, said cell is part of a mixed population of cells which present different phenotypic characteristics. In the scope of the present invention is also encompassed a cell line obtained from a transformed T-cell according to the method previously described. Modified cells resistant to an immunosuppressive treatment and susceptible to be obtained by the previous method are encompassed in the scope of the present invention.

In some embodiments, the engineered immune cells (e.g., T-cells or NK cells) are allogenic. By “allogeneic” is meant that the cells originate from a donor, from a cell line, or are produced and/or differentiated from stem cells in view of being infused into patients having a different haplotype. Such immune cells are generally engineered to be less alloreactive and/or become more persistent with respect to their patient host. More specifically, the method of engineering the allogeneic cells can comprise the step of reducing or inactivating TCR expression into T-cells, or into the stem cells to be derived into T-cells. This can be obtained by different sequence specific-reagents, such as by gene silencing or gene editing techniques by using for instance nucleases, base editing techniques, shRNA and RNAi as non-limited examples.

In some embodiments, the engineered T-cells originate from a human, wherein preferably the human is a donor, not the patient.

The engineered T-cells comprise an inactivated T-cell receptor (TCR) and have been modified by inactivating at least one component of the TCR, e.g., by using a RNA guided endonuclease associated with a specific guide RNA, or using other gene editing approaches such as TALE-nucleases. T cell receptors (TCR) are cell surface receptors that participate in the activation of T-cells in response to the presentation of antigen. The TCR is generally made from two chains, alpha and beta, which assemble to form a heterodimer and associates with the CD3-transducing subunits to form the T-cell receptor complex present on the cell surface. Each alpha and beta chain of the TCR consists of an immunoglobulin-like N-terminal variable (V) and constant (C) region, a hydrophobic transmembrane domain, and a short cytoplasmic region. As for immunoglobulin molecules, the variable region of the alpha and beta chains are generated by V (D) J recombination, creating a large diversity of antigen specificities within the population of T-cells. However, in contrast to immunoglobulins that recognize intact antigen, T-cells are activated by processed peptide fragments in association with an MHC molecule, introducing an extra dimension to antigen recognition by T-cells, known as MHC restriction. Recognition of MHC disparities between the donor and recipient through the T cell receptor leads to T-cell proliferation and the potential development of GvHD. It has been shown that normal surface expression of the TCR depends on the coordinated synthesis and assembly of all seven components of the complex (Ashwell and Klusner 1990). The inactivation of TCRalpha or TCRbeta can result in the elimination of the TCR from the surface of T-cells preventing recognition of alloantigen and thus GVHD. However, TCR disruption generally results in the elimination of the CD3 signaling component and alters the means of further T-cell expansion.

According to preferred embodiments, at least 50%, preferably at least 70%, preferably at least 90%, more preferably at least 95% of said engineered T-cells in the population are mutated in their TCRA, TCRB and/or CD3 alleles.

In some embodiments, the TCR is inactivated by using specific TALE-nucleases, better known under the trademark TALEN™ (Cellectis, 8, rue de la Croix Jarry, 75013 PARIS). This method has proven to be highly efficient in primary cells using RNA transfection as part of a platform allowing the mass production of allogeneic T-cells. See, e.g., WO 2013/176915, which is incorporated by reference herein in its entirety.

In some embodiments, the TCR is inactivated using an RNA guided endonuclease associated with a specific guide RNA. U.S. Pat. No. 10,870,864 describes methods for inactivating a TCR in cells using such methods, which is incorporated by reference herein. Engraftment of allogeneic T-cells is possible by inactivating at least one gene encoding a TCR component. In some embodiments, the TCR is rendered not functional in the cells by inactivating a TCR alpha gene and/or a TCR beta gene(s). TCR inactivation in allogeneic T-cells aims to prevent or reduce GvHD.

By inactivating a gene, it is intended that the gene of interest is not expressed in a functional protein form. In particular embodiments, genetic modification of the cells relies on the expression, in provided cells to engineer, of an RNA guided endonuclease such that it catalyzes cleavage in one targeted gene thereby inactivating the targeted gene. The nucleic acid strand breaks caused by the endonuclease are commonly repaired through the distinct mechanisms of homologous recombination or non-homologous end joining (NHEJ). However, NHEJ is an imperfect repair process that often results in changes to the DNA sequence at the site of the cleavage. Mechanisms involve rejoining of what remains of the two DNA ends through direct re-ligation (Critchlow and Jackson 1998) or via the so-called microhomology-mediated end joining (Betts, Brenchley et al. 2003; Ma, Kim et al. 2003). Repair via non-homologous end joining (NHEJ) often results in small insertions or deletions and can be used for the creation of specific gene knockouts. The modification may be a substitution, deletion, or addition of at least one nucleotide. Cells in which a cleavage-induced mutagenesis event, i.e. a mutagenesis event consecutive to an NHEJ event, has occurred can be identified and/or selected by well-known method in the art.

In some embodiments, the engineered T-cells that have been modified to express the CAR directed against FAP have one or more additional modifications. Additional genetic attributes may be conferred by gene editing T-cells in order to improve their therapeutic potency.

In some embodiments, the engineered cell can be further modified to improve its persistence or its lifespan into the patient, in particular inactivating a gene encoding MHC-I component(s) such as HLA or β2m, such as described in WO 2015/136001 or by Liu et al. (2017, Cell Res 27:154-157).

Beta-2 microglobulin, also known as β2m, is the light chain of MHC class I molecules, and as such an integral part of the major histocompatibility complex. In human, β2m is encoded by the β2m gene which is located on chromosome 15, as opposed to the other MHC genes which are located as gene cluster on chromosome 6. The human protein is composed of 119 amino acids and has a molecular weight of 11,800 Daltons. According to certain embodiments, inhibition of expression of β2m is achieved by a genome modification, more particularly through the expression in the T-cell of a rare-cutting endonuclease able to selectively inactivate by DNA cleavage the gene encoding β2m, such as the human β2m gene (NCBI Reference Sequence: NG_012920.1), or a gene having at least 70%, such as at least 80%, at least 90% at least 95%, or at least 99%, sequence identify with the human β2m gene over the entire length. Such rare-cutting endonuclease may be a TALE-nuclease, meganuclease, zing-finger nuclease (ZFN), or RNA guided endonuclease (such as Cas9).

According to certain other embodiments, inhibition of expression of β2m can be achieved by using (e.g., introducing into the T-cell) a nucleic acid molecule that specifically hybridizes (e.g. binds) under cellular conditions with the cellular mRNA and/or genomic DNA encoding β2m, thereby inhibiting transcription and/or translation of the gene. In accordance with particular embodiments, the inhibition of expression of β2m is achieved by using (e.g., introducing into the T-cell) an antisense oligonucleotide, ribozyme or interfering RNA (RNAi) molecule. Preferably, such nucleic acid molecule comprises at least 10 consecutive nucleotides of the complement of the mRNA encoding human β2m.

According to certain embodiments, a T-cell or precursor cell is provided which expresses a rare-cutting endonuclease able to selectively inactivate by DNA cleavage the gene encoding β2m. More particularly, such T-cell comprises an exogenous nucleic acid molecule comprising a nucleotide sequence encoding said rare-cutting endonuclease, which may be a TALE-nuclease, meganuclease, zing-finger nuclease (ZFN), or RNA guided endonuclease. Thus, in order to provide less alloreactive T-cells, the method of the invention can further comprise the step of inactivating or mutating one HLA gene.

In some embodiments, the engineered T-cells have been modified to suppress or repress expression of HLA in said T-cells. The class I HLA gene cluster in humans comprises three major loci, B, C and A, as well as several minor loci. The class II HLA cluster also comprises three major loci, DP, DQ and DR, and both the class I and class II gene clusters are polymorphic, in that there are several different alleles of both the class I and II genes within the population. There are also several accessory proteins that play a role in HLA functioning as well. The Tap1 and Tap2 subunits are parts of the TAP transporter complex that is essential in loading peptide antigens on to the class I HLA complexes, and the LMP2 and LMP7 proteosome subunits play roles in the proteolytic degradation of antigens into peptides for display on the HLA. Reduction in LMP7 has been shown to reduce the amount of MHC class I at the cell surface, perhaps through a lack of stabilization (Fehling et al. (1999) Science 265:1234-1237). In addition to TAP and LMP, there is the tapasin gene, whose product forms a bridge between the TAP complex and the HLA class I chains and enhances peptide loading. Reduction in tapasin results in cells with impaired MHC class I assembly, reduced cell surface expression of the MHC class I and impaired immune responses (Grandea et al. (2000) Immunity 13:213-222 and Garbi et al. (2000) Nat. Immunol. 1:234-238). Any of the above genes may be inactivated as part of the present invention as disclosed, for instance in WO 2012/012667.

In accordance with certain embodiments, the engineered T-cells are inactivated in at least one gene selected from the group consisting of RFXANK, RFX5, RFXAP, TAP1, TAP2, ZXDA, ZXDB and ZXDC. Inactivation may, for instance, be achieved by using a genome modification, more particularly through the expression in the T-cell of a rare-cutting endonuclease able to selectively inactivate by DNA cleavage a gene selected from the group consisting of RFXANK, RFX5, RFXAP, TAP1, TAP2, ZXDA, ZXDB and ZXDC. Such modifications can permit the engineered immune cells to be less alloreactive when infused into patients.

In some embodiments, said engineered T-cells have been genetically modified to suppress or repress expression of an immune checkpoint protein and/or the receptor thereof, in said T-cells, such as PD1 or CTLA4 as described in WO 2014/184744.

It will be understood by those of ordinary skill in the art, that the term “immune checkpoints” means a group of molecules expressed by T-cells. These molecules effectively serve as “brakes” to down-modulate or inhibit an immune response. Immune checkpoint molecules include, but are not limited to Programmed Death 1 (PD-1, also known as PDCD1 or CD279, accession number: NM_005018), Cytotoxic T-Lymphocyte Antigen 4 (CTLA-4, also known as CD152, GenBank accession number AF414120.1), LAG3 (also known as CD223, accession number: NM_002286.5), Tim3 (also known as HAVCR2, GenBank accession number: JX049979.1), BTLA (also known as CD272, accession number: NM_181780.3), BY55 (also known as CD160, GenBank accession number: CR541888.1), TIGIT (also known as IVSTM3, accession number: NM_173799), LAIR1 (also known as CD305, GenBank accession number: CR542051.1), SIGLEC10 (GeneBank accession number: AY358337.1), 2B4 (also known as CD244, accession number: NM_001166664.1), PPP2CA, PPP2CB, PTPN6, PTPN22, CD96, CRTAM, SIGLEC7, SIGLEC9, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL1-ORA, IL1-ORB, HMOX2, IL6R, IL6ST, EIF2AK4, CSK, PAG1, SIT1, FOXP3, PRDM1, BATF, GUCY1A2, GUCY1A3, GUCY1B2, and GUCY1B3 which directly inhibit immune cells. For example, CTLA-4 is a cell-surface protein expressed on certain CD4 and CD8 T-cells; when engaged by its ligands (B7-1 and B7-2) on antigen presenting cells, T-cell activation and effector function are inhibited. In some embodiments, the engineered T-cells and further genetically modified by inactivating at least one protein involved in the immune checkpoint, in particular PD1 and/or CTLA-4 or any immune-checkpoint proteins referred to herein.

In some embodiments, at least two genes encoding immune checkpoint proteins are inactivated, selected from the group consisting of: CTLA4, PPP2CA, PPP2CB, PTPN6, PTPN22, PDCD1, LAG3, HAVCR2, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, and GUCY1B3.

In some embodiments, the engineered T-cells can be modified or selected to confer resistance to at least one immune suppressive or chemotherapy drug, and optionally to comprise a suicide gene.

In some embodiments, the engineered T-cells cell can be further modified to confer resistance to at least one immune suppressive drug, such as by inactivating CD52 that is the target of anti-CD52 antibody (e.g.: alemtuzumab), as described for instance in WO 2013/176915.

To improve cancer therapy and selective engraftment of allogeneic T-cells, drug resistance can be conferred to the engineered T-cells to protect them from the toxic side effects of chemotherapy or immunosuppressive agents. In some embodiments, the engineered immune cell can be further modified to confer resistance to a chemotherapy drug, in particular a purine analogue drug, for example by inactivating DCK as described in WO 2015/75195.

Drug resistance of T-cells also permits their enrichment in or ex vivo, as T-cells which express a drug resistance gene, will survive and multiply relative to drug sensitive cells. In some embodiments, the methods further comprise methods of engineering allogeneic and drug resistance T-cells resistant for immunotherapy comprising: (a) providing a T-cell; (b) selecting at least one drug; (c) modifying a T-cell to confer drug resistance to said T-cell; and (d) expanding said engineered T-cell in the presence of said drug. The preceding steps may be combined with a step of modifying the T-cell by inactivating at least one gene encoding a T-cell receptor (TCR) component, and then sorting the transformed T-cells, which do not express TCR on their cell surface.

Thus, the engineered T-cells can be further modified to confer a resistance to a drug, more particularly a chemotherapy agent. The resistance to a drug can be conferred to a T-cell by expressing a drug resistance gene. Variant alleles of several genes such as dihydrofolate reductase (DHFR), inosine monophosphate dehydrogenase 2 (IMPDH2), calcineurin or methylguanine transferase (MGMT) have been identified to confer drug resistance to a cell. In some embodiments, the drug resistance gene can be expressed in the cell either by introducing a transgene encoding said gene into the cell or by integrating said drug resistance gene into the genome of the cell by homologous recombination.

The resistance to a drug can be conferred to a T-cell by inactivating one or more gene(s) responsible for the cell's sensitivity to the drug (drug sensitizing gene(s)), such as the hypoxanthine-guanine phosphoribosyl transferase (HPRT) gene (Genbank: M26434.1). In particular HPRT can be inactivated in engineered T-cells to confer resistance to a cytostatic metabolite, the 6-thioguanine (6TG) which is converted by HPRT to cytotoxic thioguanine nucleotide and which is currently used to treat patients with cancer, in particular leukemias (Hacke, Treger et al. 2013). Another example if the inactivation of the CD3 normally expressed at the surface of the T-cell can confer resistance to anti-CD3 antibodies such as teplizumab.

Otherwise, drug resistance can be conferred to the T-cell by the expression of at least one drug resistance gene. The drug resistance gene refers to a nucleic acid sequence that encodes “resistance” to an agent, such as a chemotherapeutic agent (e.g. methotrexate). In other words, the expression of the drug resistance gene in a cell permits proliferation of the cells in the presence of the agent to a greater extent than the proliferation of a corresponding cell without the drug resistance gene. A drug resistance gene of the invention can encode resistance to anti-metabolite, methotrexate, vinblastine, cisplatin, alkylating agents, anthracyclines, cytotoxic antibiotics, anti-immunophilins, their analogs or derivatives, and the like.

Several drug resistance genes have been identified that can potentially be used to confer drug resistance to targeted cells (Takebe, Zhao et al. 2001; Sugimoto, Tsukahara et al. 2003; Zielske, Reese et al. 2003; Nivens, Felder et al. 2004; Bardenheuer, Lehmberg et al. 2005; Kushman, Kabler et al. 2007).

One example of drug resistance gene can also be a mutant or modified form of Dihydrofolate reductase (DHFR). DHFR is an enzyme involved in regulating the amount of tetrahydrofolate in the cell and is essential to DNA synthesis. Folate analogs such as methotrexate (MTX) inhibit DHFR and are thus used as anti-neoplastic agents in clinic. Different mutant forms of DHFR which have increased resistance to inhibition by anti-folates used in therapy have been described. In a particular embodiment, the drug resistance gene according to the present invention can be a nucleic acid sequence encoding a mutant form of human wild type DHFR (GenBank: AAH71996.1) which comprises at least one mutation conferring resistance to an anti-folate treatment, such as methotrexate. In particular embodiment, mutant form of DHFR comprises at least one mutated amino acid at position G15, L22, F31 or F34, preferably at positions L22 or F31 ((Schweitzer, Dicker et al. 1990); International application WO94/24277; U.S. Pat. No. 6,642,043).

As used herein, “antifolate agent” or “folate analogs” refers to a molecule directed to interfere with the folate metabolic pathway at some level. Examples of antifolate agents include, e.g., methotrexate (MTX); aminopterin; trimetrexate (Neutrexin™); edatrexate; N10-propargyl-5,8-dideazafolic acid (CB3717); ZD1694 (Tumodex), 5,8-dideazaisofolic acid (IAHQ); 5,10-dideazatetrahydrofolic acid (DDATHF); 5-deazafolic acid; PT523 (N alpha-(4-amino-4-deoxypteroyl)-N delta-hemiphthaloyl-L-ornithine); 10-ethyl-10-deazaaminopterin (DDATHF, Iomatrexol); piritrexim; 10-EDAM; ZD1694; GW1843; Pemetrexate and PDX (10-propargyl-10-deazaaminopterin).

Another example of drug resistance gene can also be a mutant or modified form of ionisine-5′-monophosphate dehydrogenase II (IMPDH2), a rate-limiting enzyme in the de novo synthesis of guanosine nucleotides. The mutant or modified form of IMPDH2 is a IMPDH inhibitor resistance gene. IMPDH inhibitors can be mycophenolic acid (MPA) or its prodrug mycophenolate mofetil (MMF). The mutant IMPDH2 can comprises at least one, preferably two mutations in the MAP binding site of the wild type human IMPDH2 (NP_000875.2) that lead to a significantly increased resistance to IMPDH inhibitor. The mutations are preferably at positions T333 and/or S351 (Yam, Jensen et al. 2006; Sangiolo, Lesnikova et al. 2007; Jonnalagadda, Brown et al. 2013). In a particular embodiment, the threonine residue at position 333 is replaced with an isoleucine residue and the serine residue at position 351 is replaced with a tyrosine residue.

Another drug resistance gene is the mutant form of calcineurin. Calcineurin (PP2B) is an ubiquitously expressed serine/threonine protein phosphatase that is involved in many biological processes and which is central to T-cell activation. Calcineurin is a heterodimer composed of a catalytic subunit (CnA; three isoforms) and a regulatory subunit (CnB; two isoforms). After engagement of the T-cell receptor, calcineurin dephosphorylates the transcription factor NFAT, allowing it to translocate to the nucleus and active key target gene such as 1L2. FK506 in complex with FKBP12, or cyclosporine A (CsA) in complex with CyPA block NFAT access to calcineurin's active site, preventing its dephosphorylation and thereby inhibiting T-cell activation (Brewin, Mancao et al. 2009). The drug resistance gene of the present invention can be a nucleic acid sequence encoding a mutant form of calcineurin resistant to calcineurin inhibitor such as FK506 and/or CsA. In a particular embodiment, said mutant form can comprise at least one mutated amino acid of the wild type calcineurin heterodimer a at positions: V314, Y341, M347, T351, W352, L354, K360, preferably double mutations at positions T351 and L354 or V314 and Y341. Correspondence of amino acid positions described herein is frequently expressed in terms of the positions of the amino acids of the form of wild-type human calcineurin heterodimer (GenBank: ACX34092.1).

In another particular embodiment, said mutant form can comprise at least one mutated amino acid of the wild type calcineurin heterodimer b at positions: V120, N123, L124 or K125, preferably double mutations at positions L124 and K125. Correspondence of amino acid positions described herein is frequently expressed in terms of the positions of the amino acids of the form of wild-type human calcineurin heterodimer b polypeptide (GenBank: ACX34095.1).

Another drug resistance gene is O⁶-methylguanine methyltransferase (MGMT) encoding human alkyl guanine transferase (hAGT). AGT is a DNA repair protein that confers resistance to the cytotoxic effects of alkylating agents, such as nitrosoureas and temozolomide (TMZ). 6-benzylguanine (6-BG) is an inhibitor of AGT that potentiates nitrosourea toxicity and is co-administered with TMZ to potentiate the cytotoxic effects of this agent. Several mutant forms of MGMT that encode variants of AGT are highly resistant to inactivation by 6-BG, but retain their ability to repair DNA damage (Maze, Kurpad et al. 1999). In a particular embodiment, AGT mutant form can comprise a mutated amino acid of the wild type AGT position P140 (UniProtKB: P16455).

Another drug resistance gene can be multidrug resistance protein 1 (MDR1) gene. This gene encodes a membrane glycoprotein, known as P-glycoprotein (P-GP) involved in the transport of metabolic byproducts across the cell membrane. The P-Gp protein displays broad specificity towards several structurally unrelated chemotherapy agents. Thus, drug resistance can be conferred to cells by the expression of nucleic acid sequence that encodes MDR-1 (NP_000918).

Drug resistance genes can also be cytotoxic antibiotics, such as ble gene or mcrA gene. Ectopic expression of ble gene or mcrA in an immune cell gives a selective advantage when exposed to the chemotherapeutic agent, respectively the bleomycine or the mitomycin C.

With respect to the immunosuppressive agents, the present invention provides the possible optional steps of: (a) providing a T-cell, preferably from a cell culture or from a blood sample; (b) selecting a gene in said T-cell expressing a target for an immunosuppressive agent; (c) introducing into said T-cell RNA guided endonuclease able to selectively inactivate by DNA cleavage, preferably by double-strand break, said gene encoding a target for said immunosuppressive agent, (d) expanding said cells, optionally in presence of said immunosuppressive agent. In a more preferred embodiment, said method comprises a further step of inactivating a component of the T-cell receptor (TCR).

An immunosuppressive agent is an agent that suppresses immune function by one of several mechanisms of action. In other words, an immunosuppressive agent is a role played by a compound which is exhibited by a capability to diminish the extent and/or voracity of an immune response. As non-limiting example, an immunosuppressive agent can be a calcineurin inhibitor, a target of rapamycin, an interleukin-2α-chain blocker, an inhibitor of inosine monophosphate dehydrogenase, an inhibitor of dihydrofolic acid reductase, a corticosteroid or an immunosuppressive antimetabolite. Classical cytotoxic immunosuppressants act by inhibiting DNA synthesis. Others may act through activation of T-cells or by inhibiting the activation of helper cells. The method according to the invention allows conferring immunosuppressive resistance to T-cells for immunotherapy by inactivating the target of the immunosuppressive agent in T-cells. As non-limiting examples, targets for immunosuppressive agent can be a receptor for an immunosuppressive agent such as: CD52, glucocorticoid receptor (GR), a FKBP family gene member and a cyclophilin family gene member.

In immunocompetent hosts, allogeneic cells are normally rapidly rejected by the host immune system. It has been demonstrated that, allogeneic leukocytes present in non-irradiated blood products will persist for no more than 5 to 6 days. (Boni, Muranski et al. (2008) Blood 112 (12): 4746-54). Thus, to prevent rejection of allogeneic cells, the host's immune system must be effectively suppressed. Glucocorticoid steroids are widely used therapeutically for immunosuppression (Coutinho and Chapman (2011) Mol. Cell Endocrinol. 335 (1): 2-13). This class of steroid hormones binds to the glucocorticoid receptor (GR) present in the cytosol of T-cells resulting in the translocation into the nucleus and the binding of specific DNA motifs that regulate the expression of a number of genes involved in the immunologic process. Treatment of T-cells with glucocorticoid steroids results in reduced levels of cytokine production leading to T-cell anergy and interfering in T-cell activation. Alemtuzumab, also known as CAMPATHI-H, is a humanized monoclonal antibody targeting CD52, a 12 amino acid glycosylphosphatidyl-inositol-(GPI) linked glycoprotein (Waldmann and Hale (2005) Philos. Trans. R. Soc. Lond. B. Biol Sci. 360:1701-11). CD52 is expressed at high levels on T and B lymphocytes and lower levels on monocytes while being absent on granulocytes and bone marrow precursors. Treatment with Alemtuzumab, a humanized monoclonal antibody directed against CD52, has been shown to induce a rapid depletion of circulating lymphocytes and monocytes. It is frequently used in the treatment of T-cell lymphomas and in certain cases as part of a conditioning regimen for transplantation. However, in the case of adoptive immunotherapy the use of immunosuppressive drugs will also have a detrimental effect on the introduced therapeutic T-cells. Therefore, to effectively use an adoptive immunotherapy approach in these conditions, the introduced cells would need to be resistant to the immunosuppressive treatment.

In some embodiments, the gene that is specific for an immunosuppressive treatment is CD52, and the immunosuppressive treatment comprises a humanized antibody targeting CD52 antigen. In another embodiment, the gene that is specific for an immunosuppressive treatment is a glucocorticoid receptor (GR) and the immunosuppressive treatment comprises a corticosteroid such as dexamethasone. In another embodiment, the gene that is specific for an immunosuppressive treatment is a FKBP family gene member or a variant thereof and the immunosuppressive treatment comprises FK506 also known as Tacrolimus or fujimycin. In another embodiment, the gene that is specific for an immunosuppressive treatment is a FKBP family gene member such as FKBP12 or a variant thereof. In another embodiment, the gene that is specific for an immunosuppressive treatment is a cyclophilin family gene member or a variant thereof and the immunosuppressive treatment comprises cyclosporine.

### Immunotherapy Treatments

As provided herein, the treatment methods comprise administering to the patient an effective amount of an immunotherapy treatment that elicits an immune response in the patient.

Such immunotherapy treatment can include immune checkpoint antagonists, immune cell engagers, tumor specific vaccines (e.g. such vaccination allows expression of a tumor-specific antigen in the patient so as to raise an immune response against a tumor in said patient), and combination thereof. The invention can thus combine the use of a universal anti-FAP-CAR-expressing immune cell prepared to be active against any tumor, with that of an immunotherapy treatment specific to the patient's tumor, said immunotherapy treatment being preferably personalized, for instance by using a vaccine designed to elicit an immune response against one specific tumor antigen of said patient's tumor (i.e. a specific tumor antigen that is FAP).

### Immune Checkpoint Antagonists

In some embodiments, the immunotherapy treatment comprises administering at least one immune checkpoint antagonist to the patient.

In some embodiments, the one or more immune checkpoint inhibitors is a proteinaceous (e.g., antibody or fragment thereof, or antibody mimetic) inhibitor of PD-L1 (CD274), PD-1 (PDCD1), CTLA4, LAG-3, TIM3, TIGIT, VISTA, GITR and BTLA. In some embodiments, the one or more immune checkpoint inhibitors comprises a small organic molecule inhibitor of PD-L1 (CD274), PD-1 (PDCD1) CTLA4, LAG3, TIM3, TIGIT, VISTA, GITR or BTLA.

In some embodiments, the immune checkpoint antagonist is a CTLA4 inhibitor. In some embodiments, the inhibitor is selected from ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, HBM-4003, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

In some embodiments, the immune checkpoint antagonist is a PD-L1 (CD274) or PD-1 (PDCD1) inhibitor. In some embodiments, the inhibitor is selected from pembrolizumab, nivolumab, cemiplimab, pidilizumab, AMG-404, AMP-224, MEDI0680 (AMP-514), spartalizumab, atezolizumab, avelumab (MSB0010718C), durvalumab, BMS-936559, CK-301, PF-06801591, BGB-A317 (tislelizumab), GEN-1046 (PD-L1/4-1BB), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), RO-7247669 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGF.beta.-EC domain), CA-170 (PD-L1/VISTA), CDX-527 (CD27/PD-L1), LY-3415244 (TIM-3/PDL1), RG7769 (PD-1/TIM-3) and INBRX-105 (4-1BB/PDL1), GNS-1480 (PD-L1/EGFR), RG-7446 (Tecentriq, atezolizumab), ABBV-181, nivolumab (OPDIVO, BMS-936558, MDX-1106), pembrolizumab (KEYTRUDA, MK-3477, SCH-900475, lambrolizumab, CAS Reg. No. 1374853-91-4), pidilizumab, PF-06801591, BGB-A317 (tislelizumab), GLS-010 (WBP-3055), AK-103 (HX-008), CS-1003, HLX-10, MGA-012, BI-754091, REGN-2810 (cemiplimab), AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181, AK-105, PD1-PIK, BAT-1306, BMS-936559, atezolizumab (MPDL3280A), durvalumab (MEDI-4736), avelumab, CK-301, (MSB0010718C), MEDI-0680, CX-072, CBT-502, PDR-001 (spartalizumab), PDR001+Tafinlar®+Mekinist®, MSB-2311, JTX-4014, BGB-A333, SHR-1316, CS-1001 (WBP-3155), KN-035 (Envafolimab), IBI-308 (sintilimab), HLX-20, KL-A167, STI-A1014, STI-A1015 (IMC-001), BCD-135, FAZ-053, TQB-2450, and MDX1105-01, and those described, e.g., in WO 2018/195321, WO 2020/014643, WO 2019/160882, and WO 2018/195321.

In some embodiments, the immune checkpoint antagonist is a LAG-3 inhibitor. In some embodiments, the LAG-3 inhibitor is selected from the group consisting of relatlimab, LAG525, BMS-986016, and TSR-033.

In some embodiments, said immune checkpoint antagonist is an anti-PD1 antibody or an anti-PDL1 antibody. In some embodiments, said immune checkpoint antagonist is an anti-PD1 antibody selected from the group consisting of pembrolizumab, nivolumab, cemiplimab, and spartalizumab, or an anti-PDL1 antibody selected from the group consisting of durvalumab, atezolizumab and avelumab.

In a specific embodiment, the immune checkpoint antagonist is pembrolizumab. Pembrolizumab comprises a heavy chain of amino acid sequence SEQ ID NO: 135 and a light chain of amino acid sequence SEQ ID NO: 136.

In another specific embodiment, the immune checkpoint antagonist is nivolumab. Nivolumab comprises a heavy chain of amino acid sequence SEQ ID NO: 133 and a light chain of amino acid sequence SEQ ID NO: 134.

In some embodiments, the immune checkpoint antagonist is an anti-CTLA4 antibody that is ipilimumab. Ipilimumab comprises a heavy chain of amino acid sequence SEQ ID NO: 137 and a light chain of amino acid sequence SEQ ID NO: 138.

In some embodiments, the immune checkpoint antagonist is an anti-LAG3 antibody that is relatlimab. Relatlimab comprises a heavy chain of amino acid sequence SEQ ID NO: 139 and a light chain of amino acid sequence SEQ ID NO: 140.

Some embodiments of the invention combine more than one immune checkpoint antagonists. For instance, the immune checkpoint antagonists comprise an anti-PD1 antibody (such as nivolumab or pembrolizumab) or an anti-PDL1 antibody (such as durvalumab), and an anti-CTLA4 antibody (such as ipilimumab). In another example, the immune checkpoint antagonists comprise an anti-PD1 antibody (such as nivolumab or pembrolizumab) and an anti-LAG3 antibody (such as relatlimab).

### Immune Cell Engager

In some embodiments, the immunotherapy treatment comprises administering an effective amount of an immune cell engager comprising at least two binding sites, wherein said first binding site binds an immune cell and said second binding site binds an antigen associated with a solid tumor.

In some embodiments, the immunotherapy treatment comprises administering an effective amount of an immune cell engager and an effective amount of an immune checkpoint antagonist as described herewith.

In some embodiments, the immune cell engagers are used for the redirection of T-cells, natural killer (NK) cells and/or cytotoxic/phagocytic cells.

In a particular embodiment, the immune cell engagers comprise a first binding site binding a T-cell.

As used herein, the phrase “immune cell engager” (or “IC engager”) refers to a recombinant protein construct comprising two or more flexibly connected ligand binding domains. In some embodiments, the ligand binding domains comprise single chain antibodies (scFv). One of these ligand binding domains selectively binds at least one selected type of immune cell, such as T-cells, NK cells or APCs. The ligand binding domain preferably binds an “immune cells activating receptor” as defined below. An IC engager generally comprises a second binding domain that specifically binds a cell surface antigen, preferably an “antigen associated with a disease state,” more preferably an “antigen associated with a cancer,” which is generally chosen for being a marker of a pathological cell and for not being present at the surface of the allogeneic engineered T-cell itself. The IC engager used in the present invention preferably binds an antigen associated with a solid tumor. The function of the IC engager is to bring together selected types of immune cells with targeted malignant cells.

Various types of soluble immune cell engagers are provided in the literature as reviewed for example by Kontermann et al. (Bispecific antibodies (2015) Drug Discovery Today 20 (7): 838-847), which are suitable for the methods of the present invention. As a non-limiting list, IC engagers can be bispecific T-cell engagers (BITE), dual-affinity re-targeting antibodies (DART), bispecific engagement by antibodies based on the T-cell receptor (BEAT), CROSSMAB, TRIOMAB, tandem diabody (TANDAB), ADAPTIR, affinity-tailored adaptors for T-cells (ATAC), DUOBODY, XMAB, T-cell redirecting antibody (TRAB), BICLONICS, DUTAMAB, VELOCI-BI, hinge-mutated, bispecific antibody-armed activated T-cells (AATC), and bi-& tri-specific killer cell engagers (BIKE and TRIKE). Tetravalent heterodimeric antibodies as described in WO 2020/113164 can also be used.

“Antigen associated with a disease state” refers to an antigen present or over-expressed in a given disease. The disease can be, for instance, a cancer, in particular a solid tumor. An antigen associated with a disease state, wherein said disease state is a cancer, i.e. “an antigen associated with a cancer” can be a tumor antigen as defined herein.

The term “tumor antigen” is meant to cover “tumor-specific antigens” and “tumor associated antigens.” Tumor-Specific Antigens (TSA) are generally present only on tumor cells and not on any other cell, while Tumor-Associated Antigens (TAA) are present on some tumor cells and also present on some normal cells. “Tumor antigen,” as meant herein, also refers to mutated forms of a protein, which only appears in that form in tumors, while the non-mutated form is observed in non-tumoral tissues. A “tumor antigen” as defined herein also includes an antigen associated with the tumor microenvironment and/or the tumor stroma, such as for example VEGF present in tumor stromal fibroblasts.

In some embodiments, the immune cell engager comprises a first binding site that binds a surface antigen of a T-cell, a NK-cell, or an APC/macrophage. “Immune cell's activating receptor” refers to a receptor that triggers immune activity of immune cells, such as, preferably the TCR for T-cells, CD16 for NK cells, and CD40 for APC. In some embodiments, the specificity for the effector immune cell is able to trigger an appropriate signal transduction cascade to activate the killing machinery of the immune cell directed against the cancer cell.

In some embodiments, the immune cell engager targets T-cells. In some embodiments, the immune cell engager is a bispecific T-cell engager (BiTes). In some embodiments, the bispecific T-cell engagers comprises a tumor antigen-targeting-scFv linked with an scFv activating a specific chain of the CD3 complex (mainly the CD38 chain) that is associated with the T-cell receptor (TCR) complex and participates in TCR-mediated signaling. By utilizing this approach, T-cells are physically redirected against tumor cells and at the same time activated. The formation of this ‘artificial’ immunological synapse can be accompanied by the redistribution of signaling and secretory granule proteins in T-cells, leading to the release of perforin and granzyme. Without being bound by theory, such contact-dependent cytotoxicity is likely the main mechanism for BiTes-induced direct killing of tumor cells, as EDTA chelation of Ca2+ (required for perforin multimerization and pore formation) leads to the complete inhibition of target cell apoptosis. The activation of T-cells can also result in the secretion of cytokines and T-cell proliferation, which may be required to sustain a durable antitumor immune response. Canonical cytotoxic T-cells (CD8+ T-cells), CD4+ T-cells, γδ T-cells and NK T-cells (NKT cells) can be activated by and contribute to the antitumor activity of BiTes specific for the CD3 complex. In some embodiments, the immune cell engager can also target a co-stimulation molecule (e.g. CD28 or 4-1BB), which can be exploited to engage activated T-cells, making the immune cell engager trispecific.

In some embodiments, the first binding site binds a component of T-cell activating receptor complex (i.e. TCR), such as CD3, TCR alpha, TCR beta, TCR gamma and/or TCR delta.

In some embodiments, the first binding site binds CD3 and comprises an amino acid sequence selected from SEQ ID NO: 53 and SEQ ID NO: 60. In some embodiments, the first binding site binds CD3 and comprises an amino acid sequence having at least about 80%, 85%, 90%, 91%, 92%, 93%, 94%, 95%, 96%, 97%, 98%, or 99% identity to an amino acid sequence selected from SEQ ID NO: 53 and SEQ ID NO: 60.

In some embodiments, the first binding site binds CD3 and comprises CDRs comprising amino acids sequences of SEQ ID NO: 47 to 52 comprised in SEQ ID NO: 53. In some embodiments, the first binding site binds CD3 and comprises CDRs comprising amino acids sequences of SEQ ID NO: 54 to 59 comprised in SEQ ID NO: 60.

In some embodiments, the first binding site binds CD3 and comprises CDRs comprising amino acids sequences of SEQ ID NO: 47 to 52 and comprises an amino acid sequence having at least about 80%, 85%, 90%, 91%, 92%, 93%, 94%, 95%, 96%, 97%, 98%, or 99% identity to SEQ ID NO: 53.

In some embodiments, the first binding site binds CD3 and comprises CDRs comprising amino acids sequences of SEQ ID NO: 54 to 59 and comprises an amino acid sequence having at least about 80%, 85%, 90%, 91%, 92%, 93%, 94%, 95%, 96%, 97%, 98%, or 99% identity to SEQ ID NO: 60.

In some embodiments, the immune cell engager targets NK cells. NK cells are cytotoxic innate lymphoid cells capable of recognizing viral infected or transformed cells by a set of germline-encoded receptors, and are characterized by the lack of TCR and CD3 molecules and by the expression of CD56 (also known as neural cell adhesion molecule) and CD16 (also known as FcγRIII). NK cells activity is balanced by specific membrane receptors with activating (e.g. natural cytotoxicity receptors, like CD16) or inhibitory (e.g. inhibitory killer immunoglobulin-like receptors) functions. In some embodiments, the immune cell engager binds to CD16 on NK cells. In another embodiment, the immune cell engager binds to the activating NKG2D receptor. In some embodiments, the first binding site of the immune cell engager binds a surface antigen of a NK cell, such as a CD16 surface antigen.

In some embodiments, the immune cell engager targets cytotoxic/phagocytic immune cells (e.g., monocytes, macrophages, dendritic cells and cytokine-activated neutrophils). In some embodiments, these cells can be engaged via the non-ligand binding site of the high-affinity receptor for immunoglobulin G (FcγRI, also known as CD64) which is selectively expressed by these immune cells. In some embodiments, the first binding site of the immune cell engager binds a surface antigen such as CD40 on an antigen presenting cell. In some embodiments, the antigen presenting cell is a macrophage.

The immune cell engager comprises a second binding site that binds an antigen associated with a cancer, preferably a solid tumor antigen. The cancer antigen is not limiting. In some embodiments, the cancer antigen is selected from CEA, ERBB2, EGFR, GD2, mesothelin, MUC1, PSMA, GD2, PSMA1, LAP3, ANXA3, Tumor-associated glycoprotein 72 (TAG72), MUC16, 5T4, FRα, MUC28z, NKG2D, HRG1β, prostate stem cell antigen (PSCA), prostate-specific membrane antigen (PSMA), carboxy-anhydrase-IX (CA-IX), Trop2, claudin 18.2, folate receptor 1 (FOLR1), CXCR2, B7-H3, CD133, CD24, receptor tyrosine kinase-like orphan receptor 1-specific (ROR1), EGFRvIII, erythropoietin-producing hepatocellular carcinoma A2 (EphA2), DLL3, glypican-3, epithelial cell adhesion molecule, GUCY2C (Guanylate Cyclase 2C), and doublecortin-like kinase 1 (DCLK1), EpCAM, HER receptors HER1, HER2, HER3, HER4, PEM, A33, G250, carbohydrate antigens Le^{y}, Le^{x}, Le^{b}, STEAP1, CD166, CD24, CD44, E-cadherin, SPARC, and ErbB3. See, e.g., Marofi et al. Stem Cell Res Ther 12, 81 (2021), which is incorporated by reference herein.

In some embodiments, the antigen associated with a cancer is selected from the group consisting of mesothelin, Trop2, MUC1, EGFR, and VEGF. In preferred embodiments, the antigen is selected from Mesothelin, Trop2, and MUC1.

In some embodiments, the immune cell engager is a bispecific T-cell engager that binds to CD3 on T-cells and Trop2 on cancer cells. In some embodiments, the immune cell engager that binds to CD3 and Trop2 comprises an amino acid sequence of SEQ ID NO: 103. In some embodiments, the immune cell engager comprises an amino acid sequence having at least about 80%, 85%, 90%, 91%, 92%, 93%, 94%, 95%, 96%, 97%, 98%, or 99% identity to SEQ ID NO: 103. In some embodiments, the bispecific T-cell engager that binds to CD3 on T-cells and Trop2 on cancer cells comprises a first binding site that binds CD3 and comprises CDRs comprising amino acids sequences of SEQ ID NO: 47 to SEQ ID NO: 52; a second binding site that binds Trop2 and comprises CDRs comprising amino acids sequences of SEQ ID NO: 68 to SEQ ID NO: 73, and comprises an amino acid sequence having at least about 80%, 85%, 90%, 91%, 92%, 93%, 94%, 95%, 96%, 97%, 98%, or 99% identity to SEQ ID NO: 103 or SEQ ID NO: 74.

In some embodiments, the immune cell engager is a bispecific T-cell engager that binds to CD3 on T-cells and mesothelin on cancer cells. In some embodiments, the immune cell engager that binds to CD3 and mesothelin comprises an amino acid sequence of SEQ ID NO: 104. In some embodiments, the immune cell engager comprises an amino acid sequence having at least about 80%, 85%, 90%, 91%, 92%, 93%, 94%, 95%, 96%, 97%, 98%, or 99% identity to SEQ ID NO: 104.

In some embodiments, the bispecific T-cell engager that binds to CD3 on T-cells and mesothelin on cancer cells comprises a first binding site that binds CD3 and comprises CDRs comprising amino acids sequences of SEQ ID NO: 54 to SEQ ID NO: 59; a second binding site that binds mesothelin and comprises CDRs comprising amino acids sequences of SEQ ID NO: 82 to SEQ ID NO: 87, and comprises an amino acid sequence having at least about 80%, 85%, 90%, 91%, 92%, 93%, 94%, 95%, 96%, 97%, 98%, or 99% identity to SEQ ID NO: 104 or SEQ ID NO: 88.

In some embodiments, the immune cell engager is a bispecific T-cell engager that binds to CD3 on T-cells and MUC1 on cancer cells. In some embodiments, the immune cell engager that binds to CD3 and MUC1 comprises an amino acid sequence of SEQ ID NO: 105. In some embodiments, the immune cell engager comprises an amino acid sequence having at least about 80%, 85%, 90%, 91%, 92%, 93%, 94%, 95%, 96%, 97%, 98%, or 99% identity to SEQ ID NO: 105.

In some embodiments, the bispecific T-cell engager that binds to CD3 on T-cells and MUC1 on cancer cells comprises a first binding site that binds CD3 and comprises CDRs comprising amino acids sequences of SEQ ID NO: 54 to SEQ ID NO: 59; a second binding site that binds MUC1 and comprises CDRs comprising amino acids sequences SEQ ID NO: 75 to SEQ ID NO: 80, and comprises an amino acid sequence having at least about 80%, 85%, 90%, 91%, 92%, 93%, 94%, 95%, 96%, 97%, 98%, or 99% identity to SEQ ID NO: 105 or SEQ ID NO: 81.

In one embodiment, the bispecific T-cell engager that binds to CD3 on T-cells and Trop2 on cancer cells comprises the amino acid sequences SEQ ID NO: 53 and SEQ ID NO: 74.

In one embodiment, the bispecific T-cell engager that binds to CD3 on T-cells and Trop2 on cancer cells comprises the amino acid sequence SEQ ID NO: 103.

In one embodiment, the bispecific T-cell engager that binds to CD3 on T-cells and Mesothelin on cancer cells comprises the amino acid sequence SEQ ID NO: 60 and SEQ ID NO: 88.

In one embodiment, the bispecific T-cell engager that binds to CD3 on T-cells and Mesothelin on cancer cells comprises the amino acid sequence SEQ ID NO: 104.

In one embodiment, the bispecific T-cell engager that binds to CD3 on T-cells and MUC1 on cancer cells comprises the amino acid sequence SEQ ID NO: 60 and SEQ ID NO: 81.

In one embodiment, the bispecific T-cell engager that binds to CD3 on T-cells and MUC1 on cancer cells comprises the amino acid sequence SEQ ID NO: 105.

TABLE 4
Sequences of the CDRs comprised in the preferred BiTE ScFvs

| chain | CDR1 | CDR2 | CDR3 |
|---|---|---|---|
| CD3 | YTFTRYTMH | WIGYINPSRGYTNYNQK | YYDDHYCLDY |
| - | (SEQ ID NO: 47) | FKD | (SEQ ID NO: 49) |
| heavy | | (SEQ ID NO: 48) | |
| chain | | | |
| CD3 | TMTCRASSSVSYM | RWIYDTSKVAS | QQWSSNPLT |
| -light | N | (SEQ ID NO: 51) | (SEQ ID NO: 52) |
| chain | (SEQ ID NO: 50) | | |
| CD3a | FTFSGYGMH | SVAYITSSSINIKYADAV | FDWDKNY |
| - | (SEQ ID NO: 54) | (SEQ ID NO: 55) | (SEQ ID NO: 56) |
| heavy | | | |
| chain | | | |
| CD3a | QDISNYLN | LLIYYTNKLAD | QQYYNYPWT |
| -light | (SEQ ID NO: 57) | (SEQ ID NO: 58) | (SEQ ID NO: 59) |
| chain | | | |
| CD16 | FTFDDYGMS | WVSGINWNGGSTGYAD | GRSLLFDY |
| - | (SEQ ID NO: 61) | SV | (SEQ ID NO: 63) |
| heavy | | (SEQ ID NO: 62) | |
| chain | | | |
| CD16 | QGDSLRSYYAS | LVIYGKNNRPS | NSRDSSGNH |
| -light | (SEQ ID NO: 64) | (SEQ ID NO: 65) | (SEQ ID NO: 66) |
| chain | | | |
| Trop2 | YTFTNYGMN | MGWINTYTGEPTYT | GGFGSSYWYFDV |
| - | (SEQ ID NO: 68) | (SEQ ID NO: 69) | (SEQ ID NO: 70) |
| heavy | | | |
| chain | | | |
| Trop2 | QDVSIAVA | LLIYSASYRYT | QQHYITPLT |
| -light | (SEQ ID NO: 71) | (SEQ ID NO: 72) | (SEQ ID NO: 73) |
| chain | | | |
| MUC | NYGLS | ENHPGSGIIYHNEKFR | SSGTRGFAY |
| 1 - | (SEQ ID NO: 75) | (SEQ ID NO: 76) | (SEQ ID NO: 77) |
| heavy | | | |
| chain | | | |
| MUC | RSSQSIVHSNGNTY | LLIYKVSNRFS | FQGSHGPWT |
| 1 - | LE | (SEQ ID NO: 79) | (SEQ ID NO: 80) |
| light | (SEQ ID NO: 78) | | |
| chain | | | |
| Meso | INNNNYYWT | WIGYIYYSGSTFYNPSLK | EDTMTGLDV |
| - | | S | |
| heavy | (SEQ ID NO: 82) | (SEQ ID NO: 83) | (SEQ ID NO: 84) |
| chain | | | |
| Meso | QSINNYLN | LLIYAASSLOS | QQTYSNPT |
| -light | (SEQ ID NO: 85) | (SEQ ID NO: 86) | (SEQ ID NO: 87) |
| chain | | | |
| CD40 | FTFSDYYMY | WVAYINSGGGSTYYPDT | RGLPFHAMDY |
| - | (SEQ ID NO: 89) | V | (SEQ ID NO: 91) |
| heavy | | (SEQ ID NO: 90) | |
| chain | | | |
| CD40 | QGISNYLN | LLIYYTSILHS | QQFNKLPPT |
| -light | (SEQ ID NO: 92) | (SEQ ID NO: 93) | (SEQ ID NO: 94) |
| chain | | | |
| CD40 | YTFTSYWMH | IGNIDPSNGETHYNQKFD | ERIYYSGSTYDGYF |
| -2 - | | R | DV |
| heavy | (SEQ ID NO: 96) | (SEQ ID NO: 97) | (SEQ ID NO: 98) |
| chain | | | |
| CD40 | SSLSYMH | RWIYDTSKLAS | QQWSSNPWT |
| -2- | (SEQ ID NO: 99) | (SEQ ID NO: 100) | (SEQ ID NO: 101) |

light
chain

The cancer comprising the solid tumor is not particularly limiting. In some embodiments, the cancer expressing a tumor antigen that binds the immune cell engager is any one of breast cancer, ovarian cancer, endometrial cancer, cervical cancer, bladder cancer, renal cancer, melanoma, lung cancer, prostate cancer, testicular cancer, thyroid cancer, brain cancer, esophageal cancer, gastric cancer, pancreatic cancer, colorectal cancer, or liver cancer. All of the above listed cancers can be treated with the immune cell engagers 1) that bind to CD3 on T-cells and mesothelin; 2) that bind to CD3 on T-cells and Trop2; 3) that bind to CD3 on T-cells and MUC1; or 4) any of the immune cell engagers that are described herein and any of the cancer antigens described herein.

In some embodiments, the tumor is an ovarian cancer tumor and the antigen is selected from one or more of mesothelin, glycoprotein 72 (TAG72), MUC16, Her2, 5T4, and FRα.

In some embodiments, the tumor is a breast cancer tumor and the antigen is selected from one or more of MUC28z, NKG2D, HRG1β, and HER2.

In some embodiments, the tumor is a prostate cancer tumor and the antigen is selected from one or more of prostate stem cell antigen (PSCA) and prostate-specific membrane antigen (PSMA).

In some embodiments, the tumor is a renal cancer tumor and the antigen is carboxy-anhydrase-IX (CA-IX).

In some embodiments, the tumor is a gastric cancer tumor and the antigen is selected from one or more of Trop2, claudin18.2, NKG2D, folate receptor 1 (FOLR1), and HER2.

In some embodiments, the tumor is a pancreatic cancer tumor and the antigen is selected from one or more of mesothelin, MUC1, CXCR2, B7-H3, CD133, CD24, PSCA, CEA, and Her-2.

In some embodiments, the tumor is a lung cancer tumor and the antigen is selected from one or more of mesothelin, receptor tyrosine kinase-like orphan receptor 1-specific (ROR1), EGFRvIII, erythropoietin-producing hepatocellular carcinoma A2 (EphA2), PSCA, MUC1, and DLL3.

In some embodiments, the tumor is a liver cancer tumor and the antigen is selected from one or more of MUC1, CEA, glypican-3, and epithelial cell adhesion molecule.

In some embodiments, the tumor is a colorectal cancer tumor and the antigen is selected from one or more of MUC1, NKG2D, CD133, GUCY2C (Guanylate Cyclase 2C), TAG-72 Doublecortin-like kinase 1 (DCLK1), and CEA.

In some embodiments, the immune cell engagers are made by engineered immune cells that have been provided to the patient. In some embodiments, the immune cell engagers are made by engineered T-cells. In some embodiments, expression of T-cell receptor (TCR) is reduced or suppressed in the engineered T-cells.

In some embodiments, the immune cell engagers are administered as purified proteins to the patient.

In some embodiments, the immune cell engager is specifically directed toward the non-engineered immune cells produced by the patient. Such immune cells are preferably selected from T-cell, NK-cell, macrophage or antigen presenting cells (APC). The immune cell engager preferably binds an immune cell's activating receptor complex with the effect of activating patient's immune cells.

In some embodiments, the immune cell engagers bind at least:
CD3 and Mesothelin; and/or
CD3 and Trop2; and/or
CD3 and MUC1; and/or
CD3 and EGFR; and/or
CD3 and VEGF.

According to some embodiments, the immune cell engagers bind at least:
CD16 and Mesothelin; and/or
CD16 and Trop2; and/or
CD16 and MUC1; and/or
CD16 and EGFR; and/or
CD16 and VEGF.

According to some embodiments, the immune cell engagers bind at least:
CD40 and Mesothelin; and/or
CD40 and Trop2; and/or
CD40 and MUC1; and/or
CD40 and EGFR; and/or
CD40 and VEGF.

The immune cell engagers administered to the patient or expressed in engineered immune cells as described above that are administered to the patient preferably comprise polypeptide sequences that have at least 70%, preferably 80%, more preferably 90%, and even more preferably 95 or 99% sequence identity with those referred to in Table 5.

TABLE 5
Preferred sequences of constituents of immune cell engagers

| SEQ | IC engagers | |
|---|---|---|
| ID: # | sequence | Amino acid sequence |
| 35 | Signal Peptide IC | MYRMQLLSCIALSLALVINS |
| | engager (Trop2) | |
| 18 | CD3 scFv | DIKLQQSGAELARPGASVKMSCKTSGYTFTRYTMHWV |
| | | KQRPGQGLEWIGYINPSRGYTNYNQKFKDKATLTTDK |
| | | SSSTAYMQLSSLTSEDSAVYYCARYYDDHYCLDYWGQ |
| | | GTTLTVSSVEGGSGGSGGSGGSGGVDDIQLTQSPAIM |
| | | SASPGEKVTMTCRASSSVSYMNWYQQKSGTSPKRWIY |
| | | DTSKVASGVPYRFSGSGSGTSYSLTISSMEAEDAATY |
| | | YCQQWSSNPLTFGAGTKLELK |
| 20 | Alternative | EVQLVESGGGLVQPGKSLKLSCEASGFTFSGYGMHWV |
| | CD3 scFv | RQAPGRGLESVAYITSSSINIKYADAVKGRFTVSRDN |
| | | AKNLLFLQMNILKSEDTAMYYCARFDWDKNYWGQGTM |
| | | VTVSSGGGGSGGGGSGGGGSDIQMTQSPSSLPASLGD |
| | | RVTINCQASQDISNYLNWYQQKPGKAPKLLIYYTNKL |
| | | ADGVPSRFSGSGSGRDSSFTISSLESEDIGSYYCQQY |
| | | YNYPWTFGPGTKLEIKR |
| 36 | CD16 scFv | MEVQLVESGGGVVRPGGSLRLSCAASGFTFDDYGMSW |
| | | VRQAPGKGLEWVSGINWNGGSTGYADSVKGRFTISRD |
| | | NAKNSLYLQMNSLRAEDTAVYYCARGRSLLFDYWGQG |
| | | TLVTVSRGGGGSGGGGSGGGGSSELTQDPAVSVALGQ |
| | | TVRITCQGDSLRSYYASWYQQKPGQAPVLVIYGKNNR |
| | | PSGIPDRFSGSSSGNTASLTITGAQAEDEADYYCNSR |
| | | DSSGNHVVFGGGTKLTVL |
| 37 | Trop2 ScFv | DIQLTQSPSSLSASVGDRVSITCKASQDVSIAVAWYQQ |
| | | KPGKAPKLLIYSASYRYTGVPDRFSGSGSGTDFTLTIS |
| | | SLQPEDFAVYYCQQHYITPLTFGAGTKVEIKGGGGSGG |
| | | GGSGGGGSQVQLQQSGSELKKPGASVKVSCKASGYTFT |
| | | NYGMNWVKQAPGQGLKWMGWINTYTGEPTYTDDFKGRF |
| | | AFSLDTSVSTAYLQISSLKADDTAVYFCARGGFGSSYW |
| | | YFDVWGQGSLVTVSS |
| 38 | CD40 scFv1 | EVKLVESGGGLVQPGGSLKLSCATSGFTFSDYYMYWV |
| | | RQTPEKRLEWVAYINSGGGSTYYPDTVKGRFTISRDN |
| | | AKNTLYLQMSRLKSEDTAMYYCARRGLPFHAMDYWGQ |
| | | GTSVTVSGSTSGSGKPGSGEGSTKDIQMTQTTSSLSA |
| | | SLGDRVTISCSASQGISNYLNWYQQKPDGTVKLLIYY |
| | | TSILHSGVPSRFSGSGSGTDYSLTIGNLEPEDIATYY |
| | | CQQFNKLPPTFGGGTKLEIK |
| 39 | CD40 scFv2 | EVQLVQSGAEGVKKPGSSVKVSCKASGYTFTSYWMH |
| | | WVRQAPGQGLEWIGNIDPSNGETHYNQKFDRATLTVD |
| | | KSTSTAYMEGLSSLRSEDTAVYYCARERIYYSGSTYD |
| | | GYFDVWGQGTTVTVSSGSTSGSGKPGSGEGSTKDIQL |
| | | TQSPSPLSASVGDRVTITCSASSSLSYMHWYQQKPGK |
| | | SPKRWIYDTSKLASGVPSRFSGSGSGTEYTLTISSLQ |
| | | PEDFATYYCQQWSSNPWTFGGGTKVEIK |
| 19 | Mesothelin ScFv | QVQLQESGPGLVKPSQTLSLTCTVSGGSINNNNYYWT |
| | | WIRQHPGKGLEWIGYIYYSGSTFYNPSLKSRVTISVD |
| | | TSKTQFSLKLSSVTAADTAVYYCAREDTMTGLDVWGQ |
| | | GTTVTVSSGGGGSGGGGSGGGGSDIQMTQSPSSLSAS |
| | | VGDRVTITCRASQSINNYLNWYQQKPGKAPTLLIYAA |
| | | SSLQSGVPSRFSGSRSGTDFTLTISSLQPEDFAAYFC |
| | | QQTYSNPTFGQGTKVEVK |
| 21 | MUC1 ScFv | MEWIWIFLFILSGTAGVQSQVQLQQSGAELARPGASV |
| | | KLSCKASGYTFTNYGLSWVKQRTGQGLEWIGENHPGS |
| | | GIIYHNEKFRGKATLTADKSSSTAYVQLSSLTSEDSA |
| | | VYFCARSSGTRGFAYWGQGTLVTVSAGGGGSGGGGSG |
| | | GGGSMKLPVRLLVLMFWIPASSSDVLMTQTPLSLPVS |
| | | LGDQASISCRSSQSIVHSNGNTYLEWYLQKPGQSPKL |
| | | LIYKVSNRFSGVPDRFSGSGSGTDFTLKISRVEAEDL |
| | | GVYYCFQGSHGPWTFGGGTKLEIKRA |

In some embodiments, the immune cell engagers that can be used in the present invention comprise one or more of the bispecific antibodies shown in Table 6 below.

TABLE 6
Bispecific T-Cell Engagers directed against solid tumors (CD3 TARGETING)

| | PRODUCT | | IC | PREFERRED |
|---|---|---|---|---|
| TARGET | EXAMPLES | Manufacturers | Engager type | INDICATION(S) |
| B7H3/CD3 | MGD009 | MacroGenics | DART | Solid tumors |
| CDH3/CD3 | PF06671008 | MacroGenics & Pfizer | DART | Solid tumors |
| CEA/CD3 | AMG111 | Amgen | BiTE | Solid tumors |
| | AMG211 | | | Gastrointestinal |
| | | | | adenocarcinoma |
| | RO6958688 | Roche | CrossMab | Solid tumors, NSCLC |
| DLL3/CD3 | AMG757 | Amgen | BiTE | SCLC |
| EGFR/CD3 | EGFRBi-armed | National Cancer | AATC | Solid tumors, |
| | autologous T-cells | Institute | | pancreatic |
| | | | | adenocarcinoma, |
| | | | | lung cancer |
| EpCAM/CD3 | Solitomab | Amgen | BiTE | Epithelial cancer, |
| | | | | ovarian cancer, |
| | | | | gastric |
| | | | | adenocarcinoma, |
| | | | | malignant ascites, |
| | | | | stomach neoplasms |
| | Catumaxomab | Fresenius & Trion | TrioMab | Epithelial cancer, |
| | | Pharma | | ovarian cancer, |
| | | | | gastric |
| | | | | adenocarcinoma, |
| | | | | malignant ascites, |
| | | | | stomach neoplasms |
| GD2/CD3 | GD2Bi-armed | National Cancer | AATC | Neuroblastoma |
| | autologous T-cells | Institute | | |
| GPA33/CD3 | MGD007 | MacroGenics | DART | Colorectal cancer |
| GPC3/CD3 | ERY974 | Chugai & Roche | TRAB | Solid tumors |
| HER2/CD3 | GBR1302 | Glenmark | BEAT | Solid tumors, breast |
| | | Pharmaceuticals | | cancer |
| | Ertumaxomab | Fresenius | Triomab | Solid tumors, breast |
| | | | | cancer |
| PSCA/CD33 | GEM3PSCA | GeMoAb & Celgene | ATAC | NSCLC, breast, |
| | | | | pancreatic and |
| | | | | urogenital cancers |
| PSMA/CD3 | APVO414 | Aptevo Therapeutics | Adaptir | Prostate cancer |
| | Pasotuxizumab | Amgen | BiTE | Prostate cancer |
| SSTR2/CD3 | Tidutamab | Xencor | XmAb | Neuroendocrine & |
| | | | | gastrointestinal |
| | | | | cancers |

### Engineering and Gene Editing

The methods that can be employed herein to engineer or gene edit cells are not particularly limiting. In some embodiments, the cells are contacted with a sequence specific reagent to modify the cells. By “sequence-specific reagent” is meant any active molecule that has the ability to specifically recognize a selected polynucleotide sequence at a genomic locus, referred to as “target sequence,” which is generally of at least 9 bp, more preferably of at least 10 bp and even more preferably of at least 12 pb in length, in view of modifying the expression of said genomic locus. Said expression can be modified by mutation, deletion or insertion into coding or regulatory polynucleotide sequences, by epigenetic change, such as by methylation or histone modification, or by interfering at the transcriptional level by interacting with transcription factors or polymerases.

Examples of sequence-specific reagents are endonucleases, RNA guides, RNAi, methylases, exonucleases, histone deacetylases, endonucleases, end-processing enzymes such as exonucleases, and more particularly cytidine deaminases such as those coupled with the CRISPR/cas9 system to perform base editing (i.e. nucleotide substitution) without necessarily resorting to cleavage by nucleases as described for instance by Hess, G. T. et al. (Methods and applications of CRISPR-mediated base editing in eukaryotic genomes (2017) Mol Cell. 68 (1): 26-43) and Liu et al. (Rees, H. A. & Liu, D. R. Base editing: precision chemistry on the genome and transcriptome of living cells. Nat. Rev. Genet. 19, 770-788 (2018)).

According to one aspect, at least 50%, preferably at least 70%, preferably at least 90%, more preferably at least 95% of the cell population express a short hairpin RNA (shRNA) or small interfering (siRNA) directed against a polynucleotide sequence encoding a component of the TCR.

According to another aspect, at least 50%, preferably at least 70%, preferably at least 90%, more preferably at least 95% of the cell population express a short hairpin RNA (shRNA) or small interfering (siRNA) directed against a polynucleotide sequence encoding a component of the TCR, as well as a short hairpin RNA (shRNA) or small interfering (siRNA) directed against a polynucleotide sequence encoding β2M and/or a short hairpin RNA (shRNA) or small interfering (siRNA) directed against a polynucleotide sequence encoding CD3.

According to another embodiment of the invention, the sequence-specific reagent is preferably a sequence-specific nuclease reagent, such as an endonuclease like a rare-cutting endonuclease like TALE Nuclease, or a RNA guide coupled with a guided endonuclease like CRISPR.

The present invention aims to improve the therapeutic potential of immune cells through gene editing techniques, especially by gene targeted integration.

By “gene targeting integration” is meant any known site-specific methods allowing to insert, replace or correct a genomic coding sequence into a living cell.

According to a preferred aspect of the present invention, the gene targeted integration involves homologous gene recombination at the locus of the targeted gene to result in the insertion of, or replacement of the targeted gene by, at least one exogenous nucleotide, preferably a sequence of several nucleotides (i.e. polynucleotide), and more preferably a coding sequence.

By “DNA target,” “DNA target sequence,” “target DNA sequence,” “nucleic acid target sequence,” “target sequence,” or “processing site” is intended a polynucleotide sequence that can be targeted and processed by a sequence-specific nuclease reagent according to the present invention. These terms refer to a specific DNA location, preferably a genomic location in a cell, but also a portion of genetic material that can exist independently to the main body of genetic material such as plasmids, episomes, virus, transposons or in organelles such as mitochondria as non-limiting example. As non-limiting examples of RNA guided target sequences, are those genome sequences that can hybridize the guide RNA which directs the RNA guided endonuclease to a desired locus.

“Rare-cutting endonucleases” are sequence-specific endonuclease reagents of choice, insofar as their recognition sequences generally range from 10 to 50 successive base pairs, preferably from 12 to 30 bp, and more preferably from 14 to 20 bp.

According to a preferred aspect of the invention, said endonuclease reagent is a nucleic acid encoding an “engineered” or “programmable” rare-cutting endonuclease, such as a homing endonuclease as described for instance by Arnould S., et al. (WO2004067736), a zinc finger nuclease (ZFN) as described, for instance, by Urnov F., et al. (Highly efficient endogenous human gene correction using designed zinc-finger nucleases (2005) Nature 435:646-651), a TALE-Nuclease as described, for instance, by Mussolino et al. (A novel TALE nuclease scaffold enables high genome editing activity in combination with low toxicity (2011) Nucl. Acids Res. 39 (21): 9283-9293), or a MegaTAL nuclease as described, for instance by Boissel et al. (MegaTALs: a rare-cleaving nuclease architecture for therapeutic genome engineering (2013) Nucleic Acids Research 42 (4): 2591-2601).

According to another embodiment, the endonuclease reagent is a RNA-guide to be used in conjunction with a RNA guided endonuclease, such as Cas9 or Cpf1, as per, inter alia, the teaching by Doudna, J., and Chapentier, E., (The new frontier of genome engineering with CRISPR-Cas9 (2014) Science 346 (6213): 1077), which is incorporated herein by reference.

According to a preferred aspect of the invention, the endonuclease reagent is transiently expressed into the cells, meaning that said reagent is not supposed to integrate into the genome or persist over a long period of time, such as would be the case of RNA, more particularly mRNA, proteins or complexes mixing proteins and nucleic acids (e.g., ribonucleoproteins).

An endonuclease under mRNA form is preferably synthetized with a cap to enhance its stability according to techniques well known in the art, as described, for instance, by Kore A. L., et al. (Locked nucleic acid (LNA)-modified dinucleotide mRNA cap analogue: synthesis, enzymatic incorporation, and utilization (2009) J Am Chem Soc. 131 (18): 6364-5).

The nucleases, polynucleotides encoding these nucleases, donor polynucleotides and compositions comprising the proteins and/or polynucleotides described herein for genetically modifying the cells may be delivered in vivo or ex vivo by any suitable means.

In some embodiments, polypeptides may be synthesized in situ in a cell as a result of the introduction of polynucleotides encoding the polypeptides into the cell. In some embodiments, the polypeptides can be produced outside the cell and then introduced into the cell. Methods for introducing a polynucleotide construct into cells are known in the art and include, as non-limiting examples, stable transformation methods wherein the polynucleotide construct is integrated into the genome of the cell, transient transformation methods wherein the polynucleotide construct is not integrated into the genome of the cell and virus mediated methods. In some embodiments, the polynucleotides may be introduced into a cell by recombinant viral vectors (e.g. retroviruses, adenoviruses), liposomes and the like. For example, transient transformation methods include, for example microinjection, electroporation or particle bombardment. The polynucleotides can be included in vectors, more particularly plasmids or virus, in view of being expressed in cells.

In some embodiments, the cells are transfected with a nucleic acid encoding an endonuclease reagent. In some embodiments, 80% of the endonuclease reagent is degraded by 30 hours, preferably by 24, more preferably by 20 hours after transfection.

In some embodiments, nucleases and/or donor constructs as described herein may also be delivered using vectors containing sequences encoding one or more of the CRISPR/Cas system(s), zinc finger or TALEN protein(s).

Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more of the sequences needed for treatment. Thus, when one or more nucleases and a donor construct are introduced into the cell, the nucleases and/or donor polynucleotide may be carried on the same vector or on different vectors. When multiple vectors are used, each vector may comprise a sequence encoding one or multiple nucleases and/or donor constructs.

Conventional viral and non-viral based gene transfer methods can be used to introduce nucleic acids encoding nucleases and donor constructs in cells (e.g., mammalian cells) and target tissues.

Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6 (10): 1149-1154 (1988); Vigne, Restorative Neurology and Neuroscience 8:35-36 (1995); Kremer & Perricaudet, British Medical Bulletin 51 (1): 31-44 (1995); Haddada et al., in Current Topics in Microbiology and Immunology, Doerfler and Bohm (eds.) (1995); and Yu et al., Gene Therapy 1:13-26 (1994).

In some embodiments, methods of non-viral delivery of nucleic acids include electroporation, lipofection, microinjection, biolistics, virosomes, liposomes, immunoliposomes, polycation or lipid: nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids.

In general, electroporation steps that are used to transfect primary immune cells, such as PBMCs are typically performed in closed chambers comprising parallel plate electrodes producing a pulse electric field between said parallel plate electrodes greater than 100 volts/cm and less than 5,000 volts/cm, substantially uniform throughout the treatment volume such as described in WO 2004/083379, which is incorporated by reference, especially from page 23, line 25 to page 29, line 11. One such electroporation chamber preferably has a geometric factor (cm⁻¹) defined by the quotient of the electrode gap squared (cm2) divided by the chamber volume (cm³), wherein the geometric factor is less than or equal to 0.1 cm⁻¹, wherein the suspension of the cells and the sequence-specific reagent is in a medium which is adjusted such that the medium has conductivity in a range spanning 0.01 to 1.0 milliSiemens. In general, the suspension of cells undergoes one or more pulsed electric fields. With the method, the treatment volume of the suspension is scalable, and the time of treatment of the cells in the chamber is substantially uniform.

In some embodiments, different transgenes or multiple copies of the transgene can be included in one vector. The vector can comprise a nucleic acid sequence encoding ribosomal skip sequence such as a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see Donnelly et al., J. of General Virology 82:1013-1025 (2001); Donnelly et al., J. of Gen. Virology 78:13-21 (1997); Doronina et al., Mol. And. Cell. Biology 28 (13): 4227-4239 (2008); Atkins et al., RNA 13:803-810 (2007)).

By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA.

In one embodiment, a polynucleotide encoding a sequence specific reagent according to the present invention can be mRNA which is introduced directly into the cells, for example by electroporation. In some embodiments, the cells can be electroporated using cytoPulse technology which allows, by the use of pulsed electric fields, to transiently permeabilize living cells for delivery of material into the cells. The technology, based on the use of PulseAgile (BTX Havard Apparatus, 84 October Hill Road, Holliston, Mass. 01746, USA) electroporation waveforms grants the precise control of pulse duration, intensity as well as the interval between pulses (see U.S. Pat. No. 6,010,613 and published International Application WO 2004/083379). All these parameters can be modified in order to reach the best conditions for high transfection efficiency with minimal mortality. The first high electric field pulses allow pore formation, while subsequent lower electric field pulses allow moving the polynucleotide into the cell.

Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam and Lipofectin). Cationic and neutral lipids that are suitable for efficient receptor-recognition lipofection of polynucleotides include those of Felgner, WO 91/17424, WO 91/16024.

The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In some embodiments, the donor sequence and/or sequence specific reagent is encoded by a viral vector. In some embodiments, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Recombinant adeno-associated virus vectors (rAAV) are a promising alternative gene delivery system based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system (Wagner et al., Lancet 351:9117 1702-3 (1998), Kearns et al., Gene Ther. 9:748-55 (1996)). Other AAV serotypes, including by non-limiting example, AAV1, AAV3, AAV4, AAV5, AAV6, AAV8, AAV 8.2, AAV9, and AAV rh10 and pseudotyped AAV such as AAV2/8, AAV2/5 and AAV2/6 can also be used in accordance with the present invention.

In some embodiments, the cells are administered with an effective amount of one or more caspase inhibitors in combination with an AAV vector.

In some embodiments, the donor sequence and/or sequence specific reagent is encoded by a recombinant lentiviral vector (rLV).

The nuclease-encoding sequences and donor constructs can be delivered using the same or different systems. For example, a donor polynucleotide can be carried by a viral vector, while the one or more nucleases can be delivered as mRNA compositions.

In some embodiments, one or more reagents can be delivered to cells using nanoparticles. In some embodiments, nanoparticles are coated with ligands, such as antibodies, having a specific affinity towards HSC surface proteins, such as CD105 (Uniprot #P17813). In some embodiments, the nanoparticles are biodegradable polymeric nanoparticles in which the sequence specific reagents under polynucleotide form are complexed with a polymer of polybeta amino ester and coated with polyglutamic acid (PGA).

Due to their higher specificity, TALE-nuclease have proven to be particularly appropriate sequence specific nuclease reagents for therapeutic applications, especially under heterodimeric forms—i.e. working by pairs with a “right” monomer (also referred to as “5′” or “forward”) and ‘left” monomer (also referred to as “3″” or “reverse”) as reported for instance by Mussolino et al. (TALEN facilitate targeted genome editing in human cells with high specificity and low cytotoxicity (2014) Nucl. Acids Res. 42 (10): 6762-6773).

As previously stated, the sequence specific reagent is preferably under the form of nucleic acids, such as under DNA or RNA form encoding a rare cutting endonuclease or a subunit thereof, but they can also be part of conjugates involving polynucleotide(s) and polypeptide(s) such as so-called “ribonucleoproteins.” Such conjugates can be formed with reagents as Cas9 or Cpf1 (RNA-guided endonucleases) as respectively described by Zetsche, B. et al. (Cpf1 Is a Single RNA-Guided Endonuclease of a Class 2 CRISPR-Cas System (2015) Cell 163 (3): 759-771), which involve RNA or DNA guides that can be complexed with their respective nucleases.

“Exogenous sequence” refers to any nucleotide or nucleic acid sequence that was not initially present at the selected locus. This sequence may be homologous to, or a copy of, a genomic sequence, or be a foreign sequence introduced into the cell. By opposition “endogenous sequence” means a cell genomic sequence initially present at a locus.

As used herewith, a “donor construct” or “donor polynucleotide” comprises the exogenous nucleotide sequence to be inserted at, or replacing, the targeted gene. A donor construct can comprise a nucleotide sequence encoding a CAR described herewith, and/or an immune checkpoint antagonist and/or an immune cell engager as described herewith.

Stable expression of CARs, in particular the anti-FAP CAR described herewith, in the above-described immune cells, in particular T-cells, can be achieved using, for example, viral vectors (e.g., lentiviral vectors, retroviral vectors, Adeno-Associated Virus (AAV) vectors) or transposon/transposase systems or plasmids or PCR products integration. Other approaches include direct mRNA electroporation.

Non-limitative examples of TALE-nuclease targeting the endogenous genes expressing TRAC, CD52, and β2M are provided in Table 7. The invention can be practiced as described herein with such polynucleotides or polypeptides having at least 70%, preferably 80%, more preferably 90% and even more preferably 95 or 99% identity with the sequences referred to in Table 7.

TABLE 7
Examples of TALE-nucleases and their
target sequences

| | SEQ | |
|---|---|---|
| Targeted gene | ID # | Target sequence |
| TRAC_T01-target | 125 | TTGTCCCACAGATATCCagaaccctg |
| | | accctgCCGTGTACCAGCTGAGA |
| CD52_T02-target | 126 | TTCCTCCTACTCACCATcagcctcct |
| | | ggttatGGTACAGGTAAGAGCAA |
| B2M_T02-target | 127 | TTAGCTGTGCTCGCGCTactctctct |
| | | ttctGGCCTGGAGGCTATCCA |
| TALE-Nuclease | SEQ | TALE-Nuclease monomer |
| monomer | ID # | sequence |
| TRAC T01-L | 108 | MGDPKKKRKVIDIADLRTLGYSQQQ |
| | | QEKIKPKVRSTVAQHHEALVGHGFT |
| | | HAHIVALSQHPAALGTVAVKYQDMI |
| | | AALPEATHEAIVGVGKQWSGARALE |
| | | ALLTVAGELRGPPLQLDTGQLLKIA |
| | | KRGGVTAVEAVHAWRNALTGAPLNL |
| | | TPQQVVAIASNGGGKQALETVQRLL |
| | | PVLCQAHGLTPQQVVAIASNNGGKQ |
| | | ALETVQRLLPVLCQAHGLTPQQVVA |
| | | IASNGGGKQALETVQRLLPVLCQAH |
| | | GLTPEQVVAIASHDGGKQALETVQR |
| | | LLPVLCQAHGLTPEQVVAIASHDGG |
| | | KQALETVQRLLPVLCQAHGLTPEQV |
| | | VAIASHDGGKQALETVQRLLPVLCQ |
| | | AHGLTPEQVVAIASNIGGKQALETV |
| | | QALLPVLCQAHGLTPEQVVAIASHD |
| | | GGKQALETVQRLLPVLCQAHGLTPE |
| | | QVVAIASNIGGKQALETVQALLPVL |
| | | CQAHGLTPQQVVAIASNNGGKQALE |
| | | TVQRLLPVLCQAHGLTPEQVVAIAS |
| | | NIGGKQALETVQALLPVLCQAHGLT |
| | | PQQVVAIASNGGGKQALETVQRLLP |
| | | VLCQAHGLTPEQVVAIASNIGGKQA |
| | | LETVQALLPVLCQAHGLTPQQVVAI |
| | | ASNGGGKQALETVQRLLPVLCQAHG |
| | | LTPEQVVAIASHDGGKQALETVQRL |
| | | LPVLCQAHGLTPQQVVAIASNGGGR |
| | | PALESIVAQLSRPDPALAALTNDHL |
| | | VALACLGGRPALDAVKKGLGDPISR |
| | | SQLVKSELEEKKSELRHKLKYVPHE |
| | | YIELIEIARNSTQDRILEMKVMEFF |
| | | MKVYGYRGKHLGGSRKPDGAIYTVG |
| | | SPIDYGVIVDTKAYSGGYNLPIGQA |
| | | DEMQRYVEENQTRNKHINPNEWWKV |
| | | YPSSVTEFKFLFVSGHFKGNYKAQL |
| | | TRLNHITNCNGAVLSVEELLIGGEM |
| | | IKAGTLTLEEVRRKFNNGEINFAAD |
| TRAC T01-R | 109 | MGDPKKKRKVIDIADLRTLGYSQQQ |
| | | QEKIKPKVRSTVAQHHEALVGHGFT |
| | | HAHIVALSQHPAALGTVAVKYQDMI |
| | | AALPEATHEAIVGVGKQWSGARALE |
| | | ALLTVAGELRGPPLQLDTGQLLKIA |
| | | KRGGVTAVEAVHAWRNALTGAPLNL |
| | | TPEQVVAIASHDGGKQALETVQRLL |
| | | PVLCQAHGLTPQQVVAIASNGGGKQ |
| | | ALETVQRLLPVLCQAHGLTPEQVVA |
| | | IASHDGGKQALETVQRLLPVLCQAH |
| | | GLTPEQVVAIASNIGGKQALETVQA |
| | | LLPVLCQAHGLTPQQVVAIASNNGG |
| | | KQALETVQRLLPVLCQAHGLTPEQV |
| | | VAIASHDGGKQALETVQRLLPVLCQ |
| | | AHGLTPQQVVAIASNGGGKQALETV |
| | | QRLLPVLCQAHGLTPQQVVAIASNN |
| | | GGKQALETVQRLLPVLCQAHGLTPQ |
| | | QVVAIASNNGGKQALETVQRLLPVL |
| | | CQAHGLTPQQVVAIASNGGGKQALE |
| | | TVQRLLPVLCQAHGLTPEQVVAIAS |
| | | NIGGKQALETVQALLPVLCQAHGLT |
| | | PEQVVAIASHDGGKQALETVQRLLP |
| | | VLCQAHGLTPEQVVAIASNIGGKQA |
| | | LETVQALLPVLCQAHGLTPEQVVAI |
| | | ASHDGGKQALETVQRLLPVLCQAHG |
| | | LTPQQVVAIASNNGGKQALETVQRL |
| | | LPVLCQAHGLTPQQVVAIASNGGGR |
| | | PALESIVAQLSRPDPALAALTNDHL |
| | | VALACLGGRPALDAVKKGLGDPISR |
| | | SQLVKSELEEKKSELRHKLKYVPHE |
| | | YIELIEIARNSTQDRILEMKVMEFF |
| | | MKVYGYRGKHLGGSRKPDGAIYTVG |
| | | SPIDYGVIVDTKAYSGGYNLPIGQA |
| | | DEMQRYVEENQTRNKHINPNEWWKV |
| | | YPSSVTEFKFLFVSGHFKGNYKAQL |
| | | TRLNHITNCNGAVLSVEELLIGGEM |
| | | IKAGTLTLEEVRRKFNNGEINFAAD |
| CD52 T01-L | 110 | MGDPKKKRKVIDIADLRTLGYSQQQ |
| | | QEKIKPKVRSTVAQHHEALVGHGFT |
| | | HAHIVALSQHPAALGTVAVKYQDMI |
| | | AALPEATHEAIVGVGKQWSGARALE |
| | | ALLTVAGELRGPPLQLDTGQLLKIA |
| | | KRGGVTAVEAVHAWRNALTGAPLNL |
| | | TPQQVVAIASNGGGKQALETVQRLL |
| | | PVLCQAHGLTPEQVVAIASHDGGKQ |
| | | ALETVQRLLPVLCQAHGLTPEQVVA |
| | | IASHDGGKQALETVQRLLPVLCQAH |
| | | GLTPQQVVAIASNGGGKQALETVQR |
| | | LLPVLCQAHGLTPEQVVAIASHDGG |
| | | KQALETVQRLLPVLCQAHGLTPEQV |
| | | VAIASHDGGKQALETVQRLLPVLCQ |
| | | AHGLTPQQVVAIASNGGGKQALETV |
| | | QRLLPVLCQAHGLTPEQVVAIASNI |
| | | GGKQALETVQALLPVLCQAHGLTPE |
| | | QVVAIASHDGGKQALETVQRLLPVL |
| | | CQAHGLTPQQVVAIASNGGGKQALE |
| | | TVQRLLPVLCQAHGLTPEQVVAIAS |
| | | HDGGKQALETVQRLLPVLCQAHGLT |
| | | PEQVVAIASNIGGKQALETVQALLP |
| | | VLCQAHGLTPEQVVAIASHDGGKQA |
| | | LETVQRLLPVLCQAHGLTPEQVVAI |
| | | ASHDGGKQALETVQRLLPVLCQAHG |
| | | LTPEQVVAIASNIGGKQALETVQAL |
| | | LPVLCQAHGLTPQQVVAIASNGGGR |
| | | PALESIVAQLSRPDPALAALTNDHL |
| | | VALACLGGRPALDAVKKGLGDPISR |
| | | SQLVKSELEEKKSELRHKLKYVPHE |
| | | YIELIEIARNSTQDRILEMKVMEFF |
| | | MKVYGYRGKHLGGSRKPDGAIYTVG |
| | | SPIDYGVIVDTKAYSGGYNLPIGQA |
| | | DEMQRYVEENQTRNKHINPNEWWKV |
| | | YPSSVTEFKFLFVSGHFKGNYKAQL |
| | | TRLNHITNCNGAVLSVEELLIGGEM |
| | | IKAGTLTLEEVRRKENNGEINFAAD |
| CD52 T01-R | 111 | MGDPKKKRKVIDIADLRTLGYSQQQ |
| | | QEKIKPKVRSTVAQHHEALVGHGFT |
| | | HAHIVALSQHPAALGTVAVKYQDMI |
| | | AALPEATHEAIVGVGKQWSGARALE |
| | | ALLTVAGELRGPPLQLDTGQLLKIA |
| | | KRGGVTAVEAVHAWRNALTGAPLNL |
| | | TPQQVVAIASNGGGKQALETVQRLL |
| | | PVLCQAHGLTPQQVVAIASNNGGKQ |
| | | ALETVQRLLPVLCQAHGLTPEQVVA |
| | | IASHDGGKQALETVQRLLPVLCQAH |
| | | GLTPQQVVAIASNGGGKQALETVQR |
| | | LLPVLCQAHGLTPEQVVAIASHDGG |
| | | KQALETVQRLLPVLCQAHGLTPQQV |
| | | VAIASNGGGKQALETVQRLLPVLCQ |
| | | AHGLTPQQVVAIASNGGGKQALETV |
| | | QRLLPVLCQAHGLTPEQVVAIASNI |
| | | GGKQALETVQALLPVLCQAHGLTPE |
| | | QVVAIASHDGGKQALETVQRLLPVL |
| | | CQAHGLTPEQVVAIASHDGGKQALE |
| | | TVQRLLPVLCQAHGLTPQQVVAIAS |
| | | NGGGKQALETVQRLLPVLCQAHGLT |
| | | PQQVVAIASNNGGKQALETVQRLLP |
| | | VLCQAHGLTPQQVVAIASNGGGKQA |
| | | LETVQRLLPVLCQAHGLTPEQVVAI |
| | | ASNIGGKQALETVQALLPVLCQAHG |
| | | LTPEQVVAIASHDGGKQALETVQRL |
| | | LPVLCQAHGLTPQQVVAIASNGGGR |
| | | PALESIVAQLSRPDPALAALTNDHL |
| | | VALACLGGRPALDAVKKGLGDPISR |
| | | SQLVKSELEEKKSELRHKLKYVPHE |
| | | YIELIEIARNSTQDRILEMKVMEFF |
| | | MKVYGYRGKHLGGSRKPDGAIYTVG |
| | | SPIDYGVIVDTKAYSGGYNLPIGQA |
| | | DEMQRYVEENQTRNKHINPNEWWKV |
| | | YPSSVTEFKFLFVSGHFKGNYKAQL |
| | | TRLNHITNCNGAVLSVEELLIGGEM |
| | | IKAGTLTLEEVRRKENNGEINFAAD |
| B2M T01-L4 | 112 | MGDPKKKRKVIDIADLRTLGYSQQQ |
| | | QEKIKPKVRSTVAQHHEALVGHGFT |
| | | HAHIVALSQHPAALGTVAVKYQDMI |
| | | AALPEATHEAIVGVGKQWSGARALE |
| | | ALLTVAGELRGPPLQLDTGQLLKIA |
| | | KRGGVTAVEAVHAWRNALTGAPLNL |
| | | TPQQVVAIASNGGGKQALETVQRLL |
| | | PVLCQAHGLTPEQVVAIASNIGGKQ |
| | | ALETVQALLPVLCQAHGLTPQQVVA |
| | | IASNNGGKQALETVQRLLPVLCQAH |
| | | GLTPEQVVAIASHDGGKQALETVQR |
| | | LLPVLCQAHGLTPQQVVAIASNGGG |
| | | KQALETVQRLLPVLCQAHGLTPQQV |
| | | VAIASNNGGKQALETVQRLLPVLCQ |
| | | AHGLTPQQVVAIASNGGGKQALETV |
| | | QRLLPVLCQAHGLTPQQVVAIASNN |
| | | GGKQALETVQRLLPVLCQAHGLTPE |
| | | QVVAIASHDGGKQALETVQRLLPVL |
| | | CQAHGLTPQQVVAIASNGGGKQALE |
| | | TVQRLLPVLCQAHGLTPEQVVAIAS |
| | | HDGGKQALETVQRLLPVLCQAHGLT |
| | | PQQVVAIASNNGGKQALETVQRLLP |
| | | VLCQAHGLTPEQVVAIASHDGGKQA |
| | | LETVQRLLPVLCQAHGLTPQQVVAI |
| | | ASNNGGKQALETVQRLLPVLCQAHG |
| | | LTPEQVVAIASHDGGKQALETVQRL |
| | | LPVLCQAHGLTPQQVVAIASNGGGR |
| | | PALESIVAQLSRPDPALAALTNDHL |
| | | VALACLGGRPALDAVKKGLGDPISR |
| | | SQLVKSELEEKKSELRHKLKYVPHE |
| | | YIELIEIARNSTQDRILEMKVMEFF |
| | | MKVYGYRGKHLGGSRKPDGAIYTVG |
| | | SPIDYGVIVDTKAYSGGYNLPIGQA |
| | | DEMQRYVEENQTRNKHINPNEWWKV |
| | | YPSSVTEFKFLFVSGHFKGNYKAQL |
| | | TRLNHITNCNGAVLSVEELLIGGEM |
| | | IKAGTLTLEEVRRKENNGEINFAAD |
| B2M T01-R4 | 113 | MGDPKKKRKVIDIADLRTLGYSQQQ |
| | | QEKIKPKVRSTVAQHHEALVGHGFT |
| | | HAHIVALSQHPAALGTVAVKYQDMI |
| | | AALPEATHEAIVGVGKQWSGARALE |
| | | ALLTVAGELRGPPLQLDTGQLLKIA |
| | | KRGGVTAVEAVHAWRNALTGAPLNL |
| | | TPQQVVAIASNNGGKQALETVQRLL |
| | | PVLCQAHGLTPQQVVAIASNNGGKQ |
| | | ALETVQRLLPVLCQAHGLTPEQVVA |
| | | IASNIGGKQALETVQALLPVLCQAH |
| | | GLTPQQVVAIASNGGGKQALETVQR |
| | | LLPVLCQAHGLTPEQVVAIASNIGG |
| | | KQALETVQALLPVLCQAHGLTPQQV |
| | | VAIASNNGGKQALETVQRLLPVLCQ |
| | | AHGLTPEQVVAIASHDGGKQALETV |
| | | QRLLPVLCQAHGLTPEQVVAIASHD |
| | | GGKQALETVQRLLPVLCQAHGLTPQ |
| | | QVVAIASNGGGKQALETVQRLLPVL |
| | | CQAHGLTPEQVVAIASHDGGKQALE |
| | | TVQRLLPVLCQAHGLTPEQVVAIAS |
| | | HDGGKQALETVQRLLPVLCQAHGLT |
| | | PEQVVAIASNIGGKQALETVQALLP |
| | | VLCQAHGLTPQQVVAIASNNGGKQA |
| | | LETVQRLLPVLCQAHGLTPQQVVAI |
| | | ASNNGGKQALETVQRLLPVLCQAHG |
| | | LTPEQVVAIASHDGGKQALETVQRL |
| | | LPVLCQAHGLTPQQVVAIASNGGGR |
| | | PALESIVAQLSRPDPALAALTNDHL |
| | | VALACLGGRPALDAVKKGLGDPISR |
| | | SQLVKSELEEKKSELRHKLKYVPHE |
| | | YIELIEIARNSTQDRILEMKVMEFF |
| | | MKVYGYRGKHLGGSRKPDGAIYTVG |
| | | SPIDYGVIVDTKAYSGGYNLPIGQA |
| | | DEMQRYVEENQTRNKHINPNEWWKV |
| | | YPSSVTEFKFLFVSGHFKGNYKAQL |
| | | TRLNHITNCNGAVLSVEELLIGGEM |
| | | IKAGTLTLEEVRRKENNGEINFAAD |
| TGFBR2_L | 141 | MGDPKKKRKVIDIADLRTLGYSQQQ |
| | | QEKIKPKVRSTVAQHHEALVGHGFT |
| | | HAHIVALSQHPAALGTVAVKYQDMI |
| | | AALPEATHEAIVGVGKQWSGARALE |
| | | ALLTVAGELRGPPLQLDTGQLLKIA |
| | | KRGGVTAVEAVHAWRNALTGAPLNL |
| | | TPEQVVAIASHDGGKQALETVQRLL |
| | | PVLCQAHGLTPEQVVAIASHDGGKQ |
| | | ALETVQRLLPVLCQAHGLTPEQVVA |
| | | IASHDGGKQALETVQRLLPVLCQAH |
| | | GLTPQQVVAIASNGGGKQALETVQR |
| | | LLPVLCQAHGLTPEQVVAIASNIGG |
| | | KQALETVQALLPVLCQAHGLTPQQV |
| | | VAIASNGGGKQALETVQRLLPVLCQ |
| | | AHGLTPQQVVAIASNNGGKQALETV |
| | | QRLLPVLCQAHGLTPEQVVAIASNI |
| | | GGKQALETVQALLPVLCQAHGLTPQ |
| | | QVVAIASNNGGKQALETVQRLLPVL |
| | | CQAHGLTPQQVVAIASNNGGKQALE |
| | | TVQRLLPVLCQAHGLTPEQVVAIAS |
| | | NIGGKQALETVQALLPVLCQAHGLT |
| | | PQQVVAIASNNGGKQALETVQRLLP |
| | | VLCQAHGLTPQQVVAIASNGGGKQA |
| | | LETVQRLLPVLCQAHGLTPEQVVAI |
| | | ASNIGGKQALETVQALLPVLCQAHG |
| | | LTPQQVVAIASNGGGKQALETVQRL |
| | | LPVLCQAHGLTPQQVVAIASNGGGR |
| | | PALESIVAQLSRPDPALAALTNDHL |
| | | VALACLGGRPALDAVKKGLGDPISR |
| | | SQLVKSELEEKKSELRHKLKYVPHE |
| | | YIELIEIARNSTQDRILEMKVMEFF |
| | | MKVYGYRGKHLGGSRKPDGAIYTVG |
| | | SPIDYGVIVDTKAYSGGYNLPIGQA |
| | | DEMQRYVEENQTRNKHINPNEWWKV |
| | | YPSSVTEFKFLFVSGHFKGNYKAQL |
| | | TRLNHITNCNGAVLSVEELLIGGEM |
| | | IKAGTLTLEEVRRKFNNGEINFAAD |
| TGFBR2_R | 142 | MGDPKKKRKVIDIADLRTLGYSQQQ |
| | | QEKIKPKVRSTVAQHHEALVGHGFT |
| | | HAHIVALSQHPAALGTVAVKYQDMI |
| | | AALPEATHEAIVGVGKQWSGARALE |
| | | ALLTVAGELRGPPLQLDTGQLLKIA |
| | | KRGGVTAVEAVHAWRNALTGAPLNL |
| | | TPQQVVAIASNNGGKQALETVQRLL |
| | | PVLCQAHGLTPEQVVAIASNIGGKQ |
| | | ALETVQALLPVLCQAHGLTPQQVVA |
| | | IASNNGGKQALETVQRLLPVLCQAH |
| | | GLTPEQVVAIASNIGGKQALETVQA |
| | | LLPVLCQAHGLTPEQVVAIASNIGG |
| | | KQALETVQALLPVLCQAHGLTPQQV |
| | | VAIASNNGGKQALETVQRLLPVLCQ |
| | | AHGLTPEQVVAIASNIGGKQALETV |
| | | QALLPVLCQAHGLTPQQVVAIASNG |
| | | GGKQALETVQRLLPVLCQAHGLTPQ |
| | | QVVAIASNNGGKQALETVQRLLPVL |
| | | CQAHGLTPQQVVAIASNGGGKQALE |
| | | TVQRLLPVLCQAHGLTPEQVVAIAS |
| | | HDGGKQALETVQRLLPVLCQAHGLT |
| | | PEQVVAIASHDGGKQALETVQRLLP |
| | | VLCQAHGLTPQQVVAIASNGGGKQA |
| | | LETVQRLLPVLCQAHGLTPQQVVAI |
| | | ASNGGGKQALETVQRLLPVLCQAHG |
| | | LTPEQVVAIASHDGGKQALETVQRL |
| | | LPVLCQAHGLTPQQVVAIASNGGGR |
| | | PALESIVAQLSRPDPALAALTNDHL |
| | | VALACLGGRPALDAVKKGLGDPISR |
| | | SQLVKSELEEKKSELRHKLKYVPHE |
| | | YIELIEIARNSTQDRILEMKVMEFF |
| | | MKVYGYRGKHLGGSRKPDGAIYTVG |
| | | SPIDYGVIVDTKAYSGGYNLPIGQA |
| | | DEMQRYVEENQTRNKHINPNEWWKV |
| | | YPSSVTEFKFLFVSGHFKGNYKAQL |
| | | TRLNHITNCNGAVLSVEELLIGGEM |
| | | IKAGTLTLEEVRRKFN |
| | | NGEINFAAD |

In some embodiments, the exogenous polynucleotide sequences for expression of the anti-FAP CAR, can be integrated at a locus regulated by or encoding TCR, HLA, β2m, PD1, CTLA4, TIM3, LAG3, CD69, IL2Ra and/or CD52. As a consequence, in these embodiments, the targeted gene's expression is reduced or suppressed.

In some embodiments, the vector can comprise an exogenous sequence coding for a chimeric receptor, for instance an anti-FAP chimeric antigen receptor (CAR), which is optionally co-expressed with an immune checkpoint antagonist or an immune cell engager.

Gene targeted insertion of the sequences encoding CARs and/or other exogenous genetic sequences can be performed by using AAV vectors, especially vectors from the AAV6 family or chimeric vectors AAV2/6 previously described by Sharma A., et al. (Transduction efficiency of AAV 2/6, 2/8 and 2/9 vectors for delivering genes in human corneal fibroblasts. (2010) Brain Research Bulletin. 81 (2-3): 273-278).

One aspect of the present invention is thus the transduction of such AAV vectors encoding a CAR, in particular an anti-FAP CAR as described herewith, in human primary T-cells, in conjunction with the expression of sequence-specific endonuclease reagents, such as TALE endonucleases, to increase gene integration at the loci previously cited.

Another aspect of the present invention is the transduction of a recombinant lentiviral vector (rLV) encoding a CAR, in particular an anti-FAP CAR as described herewith, in human primary T-cells, that can be performed before or after introduction of a sequence-specific endonuclease reagent, such as a TALE endonuclease, to inactivate the genes previously cited (e.g. TCR, HLA, β2m, PD1, CTLA4, TIM3, LAG3, CD69, IL2Ra and/or CD52).

According to a preferred aspect of this invention, sequence specific endonuclease reagents can be introduced into the cells by transfection, more preferably by electroporation of mRNA encoding said sequence specific endonuclease reagents.

Accordingly, the invention provides a method for inserting an exogenous nucleic acid sequence coding for a CAR, in particular an anti-FAP CAR as described herein, at one of the previously cited locus, which comprises at least one of the following steps:
transducing into said cell an AAV vector comprising an exogenous nucleic acid sequence encoding an anti-FAP CAR and the sequences homologous to the targeted endogenous DNA sequence, and optionally:
inducing the expression of a sequence specific endonuclease reagent to cleave said endogenous sequence at the locus of insertion.

The obtained insertion of the exogenous nucleic acid sequence may result into the introduction of genetic material and replacement of the endogenous sequence, and, thus, inactivation of the endogenous locus.

As one object of the present invention, the AAV vector used in the method can comprise an exogenous coding sequence that is “promoterless,” the coding sequence being any of those referred to in this specification.

Many other vectors known in the art, such as plasmids, episomal vectors, linear DNA matrices, etc. can also be used to perform gene insertions at those loci by following the teachings of the present invention.

As stated before, the DNA vector used for gene integration according to the invention preferably comprises: (1) the exogenous nucleic acid to be inserted comprising the exogenous coding sequence of an anti-FAP CAR as described herewith, and (2) a sequence encoding the sequence specific endonuclease reagent that promotes the insertion. According to a more preferred aspect, said exogenous nucleic acid under (1) does not comprise any promoter sequence, whereas the sequence under (2) has its own promoter.

According to another aspect, when said anti-FAP CAR is a multi-chain CAR, the nucleic acid under (1) further comprises an Internal Ribosome Entry Site (IRES) or “self-cleaving” 2A peptides, such as T2A, P2A, E2A or F2A, so that the exogenous coding sequence inserted is multi-cistronic. The IRES of 2A Peptide can precede or follow said exogenous coding sequence.

The integration of the exogenous polynucleotide sequences for expression of said anti-FAP CAR can also be introduced into the T-cells by using a viral vector, in particular lentiviral vectors. The present invention thus provides with viral vectors encoding anti-FAP CARs as described herein.

In some embodiments, lentiviral or AAV vectors according to the invention can comprise sequences encoding different elements of an anti-FAP CAR separated by a T2A or P2A sequence, as forming one transcriptional unit. In lentiviral vectors said sequences generally form an expression cassette transcribed under control of a constitutive exogenous promoter, such as a EF1alpha promoter derived from the human EEF1A1 gene.

### Activation and Expansion of T-Cells

Whether prior to or after genetic modification, the immune cells according to the present invention can be activated or expanded, even if they can activate or proliferate independently of antigen binding mechanisms. T-cells, in particular, can be activated and expanded using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and 7,572,631. T-cells can be expanded in vitro or in vivo. T-cells are generally expanded by contact with an agent that stimulates a CD3 TCR complex and a co-stimulatory molecule on the surface of the T-cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T-cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T-cells, a ligand that binds the accessory molecule is used. For example, a population of T-cells can be contacted with an anti-CD3 antibody and an anti-CD28 antibody, under conditions appropriate for stimulating proliferation of the T-cells. Conditions appropriate for T-cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, IL-4, IL-7, GM-CSF, IL-10, IL-12, IL-15, TGFp, and TNF- or any other additives for the growth of cells known to the skilled artisan. Other additives for the growth of cells include, but are not limited to, surfactant, plasmanate, and reducing agents such as N-acetyl-cysteine and 2-mercaptoethanoi. Media can include RPMI 1640, A1M-V, DMEM, MEM, a-MEM, F-12, X-Vivo 1, and X-Vivo 20, OptTmizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T-cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02). T-cells that have been exposed to varied stimulation times may exhibit different characteristics.

In another particular embodiment, said cells can be expanded by co-culturing with tissue or cells. Said cells can also be expanded in vivo, for example in the subject's blood after administrating said cell into the subject.

Any biological activity exhibited by the engineered immune cell expressing a CAR can be determined, including, for instance, cytokine production and secretion, degranulation, proliferation, or any combination thereof.

In a particular instance, the biological activity determined in step (iii) is cytokine secretion, cell proliferation, or both.

The biological activities can be measured by standard methods well known by the skilled person, in particular by in vitro and/or ex vivo methods.

Secretion of any cytokine can be measured, in particular secretion of IFNγ, TNFα, can be determined. Standard methods to determine cytokine secretion includes ELISA, flow cytometry. These methods are described for instance in Sachdeva et al. (Front Biosci, 2007, 12:4682-95) and Pike et al (2016) (Methods in Molecular Biology, vol 1458. Humana Press, New York, NY).

The level of cytokine secretion can be measured, for instance, as the maximum level of cytokine (e.g., IFNγ) secreted per CAR-expressing immune cell (e.g., CAR-T cell), e.g. maximum amount of IFNγ secreted per CAR-T cell.

To evaluate “degranulation,” standard methods can be used, including for instance CD107a degranulation assay or measurement of secreted Granzyme B or Perforin (such as described in Lorenzo-Herrero et al, [Methods Mol Biol (2019) 1884:119-130; Betts et al. Methods in Cell Biology (2004) 75:497-512].

To evaluate “proliferation” activity, standard methods can be carried out, which are mainly based on methods involving measurement of DNA synthesis, detection of proliferation-specific markers, measurement of successive cell divisions by the use of cell membrane binding dyes, measurement of cellular DNA content and measurement of cellular metabolism.

In some embodiments, the methods of the present invention allow producing engineered T-cells within a limited time frame of about 15 to 30 days, preferably between 15 and 20 days, and most preferably between 18 and 20 days so that the cells keep their full immune therapeutic potential, especially with respect to their cytotoxic activity.

These cells can be from or be members of populations of cells, which preferably originate from a single donor or patient. In some embodiments, these populations of cells can be expanded under closed culture recipients to comply with highest manufacturing practices requirements and can be frozen prior to infusion into a patient, thereby providing “off the shelf” or “ready to use” therapeutic compositions.

In some embodiments, a significant number of cells originating from the same leukapheresis can be obtained, which can be important to obtain sufficient doses for treating a patient. Although variations between populations of cells originating from various donors may be observed, the number of immune cells procured by a leukapheresis is generally about from 10⁸ to 10¹⁰ cells of PBMC. PBMC comprises several types of cells: granulocytes, monocytes and lymphocytes, among which from 30 to 60% of T-cells, which generally represents between 10⁸ to 10⁹ of primary T-cells from one donor.

In some embodiments, methods of the present invention generally end up with a population of engineered cells that reaches generally more than about 10⁸ T-cells, more generally more than about 10⁹ T-cells, even more generally more than about 10¹⁰ T-cells, and usually more than 10¹¹ T-cells. In some embodiments, the T-cells are gene edited in at least at two different loci.

Such compositions or populations of engineered cells can therefore be used as a therapeutic; especially for treating any of the cancers herein, particularly for the treatment of solid tumors in patients such as melanomas, neuroblastomas, gliomas or carcinomas such as lung, breast, colon, prostate or ovary tumors in a patient in need thereof.

The invention is more particularly drawn to populations of primary TCR negative T-cells originating from a single donor, wherein at least 20%, preferably 30%, more preferably 50% of the cells in said population have been modified using sequence-specific reagents in at least two, preferably three different loci.

By “TCR negative immune cell” is meant an immune cell, preferably T cell or NK cell, in which expression of TCR is either absent naturally (i.e. without having to engineer the cell for making this cell TCR negative) or is reduced by at least 50% compared to a non-engineered cell if the cell has been engineered to become TCR negative. TCR negative immune cells include immune cells which have at least one of the endogenous allele encoding a component of the T-cell receptor that has been genetically modified (e.g., disrupted), so that TCR expression in said engineered cell is repressed or suppressed. TCR negative immune cells also include immune cells which, in their natural non-engineered state, generally do not express TCR gene, such as is the case of NK cells.

The treatments involving the engineered primary immune cells according to the present invention can be ameliorating, curative or prophylactic.

In some embodiments, the patient can undergo preparative lymphodepletion—the temporary ablation of the immune system-prior to administration of the engineered T-cells. In some embodiments, the lymphodepletion is only partial and not a complete ablation of the patient's immune system. In some embodiments, a combination of IL-2 treatment and preparative lymphodepletion can enhance persistence of a cellular therapeutic.

In some embodiments, the engineered anti-FAP CAR T-cells can be administered in an amount of about 10⁶ to 10⁹ cells/kg, with or without a course of lymphodepletion, for example by administering cyclophosphamide.

In some embodiments, the cells or population of cells comprising the engineered anti-FAP CAR T-cells described herewith are administered in an amount of about 10⁴-10⁹ cells per kg body weight, preferably 10⁵ to 10⁶ cells/kg body weight including all integer values of cell numbers within those ranges. Dosing in CAR-T cell therapies may for example involve administration of from 10⁵ or 10⁶ to 10⁹ cells/kg, with or without a course of lymphodepletion, for example with cyclophosphamide.

The cells or population of cells can be administered in one or more doses. In another embodiment, the effective amount of cells are administered as a single dose. In another embodiment, the effective amount of cells are administered as more than one dose over a period of time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art.

In some embodiments, the immune checkpoint antagonist can be administered intravenously in an amount of about 200 mg to 400 mg including all integer values within those ranges. The immune checkpoint antagonist can be administered in one or more doses. In an embodiment, the effective amount of immune checkpoint antagonist is administered as a single dose. In another embodiment, the effective amount immune checkpoint antagonist is administered as more than one dose over a period of time. Timing of administration is within the judgment of managing physician and depends on the clinical condition of the patient. Administration of the immune checkpoint antagonist may start 1 or 2 weeks after administration of the CAR-T cells, such as between about 1 or 2 weeks and about 3 to 10 months, between 2 weeks and 8 months, or between 2 weeks and 4 months after administration of the CAR-T cells. The immune checkpoint antagonist may be administered as a purified protein or indirectly by administering an engineered cell expressing said immune checkpoint antagonist. While individual needs vary, determination of optimal ranges of effective amounts of checkpoint antagonist, or engineered cell expressing thereof, for a particular disease or conditions are within the skill of one in the art.

In some embodiments, the immune cell engager can be administered at a dose of about 10 to 50 microgram per day including all integer values within those ranges, e.g. about 30 microgram/day, as continuous intravenous infusion at constant flow rate for a time period. The immune cell engager can be administered in one or more doses. In an embodiment, the effective amount of immune cell engager is administered as a single dose. In another embodiment, the effective amount of immune cell engager is administered as more than one dose over a period of time. Timing of administration is within the judgment of managing physician and depends on the clinical condition of the patient. Administration of the immune cell engager may start 1 or 2 weeks after administration of the CAR-T cells, such as between about 1 or 2 weeks and about 3 to 10 months, between 2 weeks and 8 months, or between 2 weeks and 4 months after administration of the CAR-T cells.

The immune cell engager may be administered as a purified protein or indirectly by administering an engineered cell expressing said immune cell engager. While individual needs vary, determination of optimal ranges of effective amounts of immune cell engager, or engineered cell expressing thereof, for a particular disease or conditions are within the skill of one in the art.

An effective amount of CAR-T cells, immune checkpoint antagonist or immune cell engager, means an amount which provides a therapeutic or prophylactic benefit. The dosage administered will be dependent upon the age, health and weight of the recipient, kind of concurrent treatment, if any, frequency of treatment and the nature of the effect desired.

The combined treatment with the engineered T-cells and the immunotherapy according to the invention may be carried out in further combination with one or more therapies against cancer selected from the group of antibodies therapy, chemotherapy, cytokines therapy, dendritic cell therapy, gene therapy, hormone therapy, laser light therapy and radiation therapy.

What is described herewith with engineered T-cells comprising an inactivated TCR and expressing a FAP-CAR can equally be applied to engineered Natural Killer cells expressing a FAP-CAR.

Such engineered NK cells are naturally TCR negative. The NK cells according to the invention originate from a donor or from a cell line such as NK92 cell line.

Optionally, said engineered NK cells have a reduced expression of β2M gene mediated by gene inactivation and/or by gene silencing and/or by inserting into the β2M locus of said NK-cells' genome at least one exogenous polynucleotide encoding a CAR as defined herewith.

Said engineered NK cells may have a reduced expression of CD52 gene mediated by gene inactivation and/or by gene silencing and/or by inserting into the CD52 locus of said NK-cells' genome at least one exogenous polynucleotide encoding a CAR as defined herewith.

In one embodiment, said engineered NK cells comprise either the CD52 or the β2M gene inactivated.

Thus, is also provided herewith an engineered NK-cell expressing at its cell surface a Chimeric Antigen Receptor (CAR) directed against Fibroblast Activation Protein (FAP), wherein the CAR comprises:
(a) an extracellular ligand binding-domain comprising VH and VL amino acid sequences from a monoclonal anti-FAP antibody,
(b) a hinge amino acid sequence selected from a FcγRIII hinge, a CD8α hinge and an IgG1 hinge,
(c) a transmembrane domain amino acid sequence comprising a CD8α transmembrane domain or a CD28 transmembrane domain, and
(d) a cytoplasmic domain comprising amino acid sequences from a CD3 zeta signaling domain and a co-stimulatory domain from 4-1BB or from CD28;
wherein, optionally, the NK-cell has been genetically modified to suppress or repress expression of at least one MHC protein, preferably β2m or HLA, in the NK-cell.

Similar FAP-CARs as described herewith can be expressed in said NK cells to produce engineered CAR-NK-FAP, which can be used in methods of treatment of a solid tumor in combination with immunotherapy treatment that elicits an immune response in the patient as described herewith.

Thus, are also described herewith a pharmaceutical composition comprising (i) engineered NK-cells, optionally comprising an inactivated β2M gene, and expressing at their cell surface a Chimeric Antigen Receptor (CAR) directed against Fibroblast Activation Protein (FAP) (UCARNK-FAP), and (ii) an immunotherapy treatment for eliciting an immune response in a patient, wherein both components (i) and (ii) are formulated for separate administration.

A composition comprising engineered NK-cells, optionally comprising an inactivated β2M gene, and expressing at their cell surface a Chimeric Antigen Receptor (CAR) directed against Fibroblast Activation Protein (FAP) (UCARNK-FAP) for use in the treatment of a solid tumor in a patient in need thereof, wherein said engineered NK-cells are administered in combination with an immunotherapy treatment for eliciting an immune response in said patient.

A composition comprising an immunotherapy treatment for eliciting an immune response in a patient for use in the treatment of a solid tumor in said patient, wherein said immunotherapy treatment is administered in combination with engineered NK-cells, optionally comprising an inactivated β2M gene, and expressing at their cell surface a Chimeric Antigen Receptor (CAR) directed against Fibroblast Activation Protein (FAP) (UCARNK-FAP).

The above written description of the invention provides a manner and process of making and using it such that any person skilled in this art is enabled to make and use the same, this enablement being provided in particular for the subject matter of the appended claims, which make up a part of the original description.

Where a numerical limit or range is stated herein, the endpoints are included. Also, all values and subranges within a numerical limit or range are specifically included as if explicitly written out.

Having generally described this invention, a further understanding can be obtained by reference to certain specific examples, which are provided herein for purposes of illustration only, and are not intended to limit the scope of the claimed invention.

### EXAMPLES

### Example 1. B2M^{KO} UCART-FAP Cell Production by Lentiviral Transduction

In this example it is shown that primary T cells can be transfected with TALEN to knockout TRAC and β2M genes and transduced with lentivirus to express CAR against FAP protein.

### Expression of FAP-CAR

To express a FAP-CAR on the surface of primary T cells, cryopreserved PBMC were thawed at 37° C., washed and re-suspended in OpTmizer medium supplemented with AB human serum (5%) for overnight incubation at 37° C. in 5% CO₂ incubator. Cells were then activated with Transact in OpTmizer medium supplemented with AB human serum (5%) and recombinant human interleukin-2 (rhIL-2, 350 IU/mL) in a CO₂ incubator (culture medium). Three days after activation, T cells were transduced with lentiviral particle containing a nucleotide sequence encoding an anti-FAP CAR of SEQ ID NO: 10 at an MOI of 15. The nucleotide sequence used in this example was SEQ ID NO: 129.

### Knockout of TRAC and β2M

Two days after transduction, anti-FAP-CAR-T cells were electroporated with 5 μg of mRNAs encoding TRAC TALEN® arms (SEQ ID NO: 108 and SEQ ID NO: 109) and 5 μg of mRNAs encoding β2M TALEN® arms (SEQ ID NO: 112 and SEQ ID NO: 113). Transfection was performed using Pulse Agile technology by applying two 0.1 mS pulses at 800V followed by four 0.2 mS pulses at 130 V in 0.4 cm gap cuvettes in Cytoporation buffer T (BTX Harvard Apparatus, Holliston, Massachusetts). The electroporated cells were then immediately transferred into prewarmed Optmizer serum-free media and incubated at 37° C. for 15 min and then at 30° C. for 16 h. Cells were thereafter cultivated at 37° C. in the presence of 5% CO₂.

The cells were analyzed for anti-FAP-CAR expression and TRAC and β2M knockout five days later.

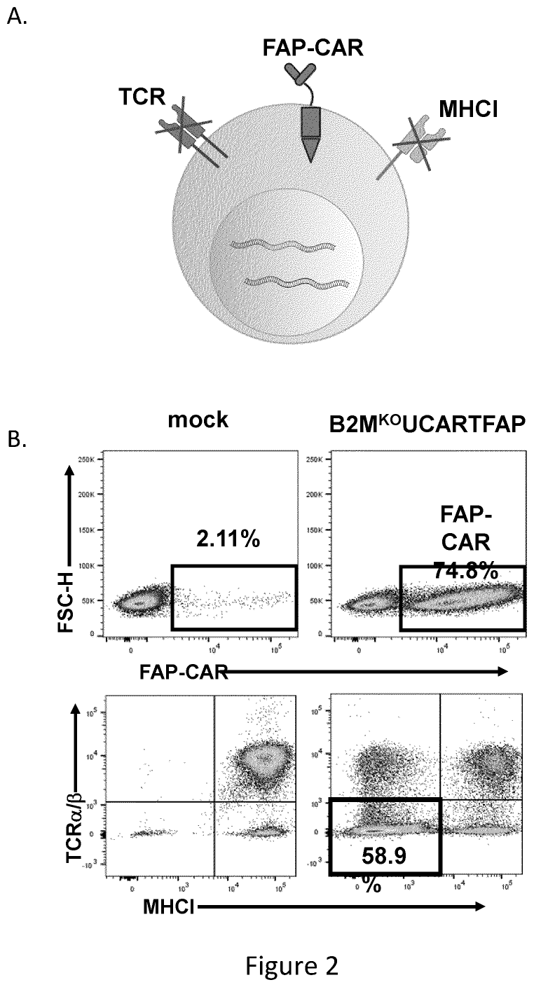

The results showed that engineered β2M^{KO}UCART-FAP cells expressed anti-FAP CAR and were deficient in expression of TCRα/β and HLA-ABC, whereas no CAR expression or gene knockout could be detected in mock transfected T cells (FIG. 2).

### Example 2. UCART-FAP Cell Production by AAV6 Transduction

In this example it is shown that primary T cells can be transfected with TALEN to knockout TRAC and can be transduced with AAV6 to express a CAR against FAP protein.

### Knockout of TRAC and Targeted Expression of Anti-FAP-CAR

Cryopreserved PBMC were thawed at 37° C., washed and re-suspended in OpTmizer medium supplemented with AB human serum (5%) for overnight incubation at 37° C. in 5% CO₂ incubator. Cells were then activated with Transact in OpTmizer medium supplemented with AB human serum (5%) and recombinant human interleukin-2 (rhIL-2, 350 IU/mL) in a CO₂ incubator (culture medium). Three days after activation the amplified T-cells were electroporated with the 5 μg of mRNAs encoding TRAC TALEN arms (SEQ ID NO: 108 and SEQ ID NO: 109). Transfection was performed using Pulse Agile technology by applying two 0.1 mS pulses at 800 V followed by four 0.2 mS pulses at 130 V in 0.4 cm gap cuvettes in Cytoporation buffer T (BTX Harvard Apparatus, Holliston, Massachusetts). The electroporated cells were then immediately transferred into prewarmed OpTmizer serum-free media and incubated at 37° C. for 15 min and for another 15 min at 30° C. The cells were then concentrated and incubated in the presence of AAV6 particles (MOI=2.5E5 vg/cells) comprising one of the donor matrices depicted in FIG. 2. These donor matrices are composed of 300 bp of the TRAC left and right Homology arms, a self-cleaving 2A peptide allowing the expression of the anti-FAP CAR of SEQ ID NO: 10). After 2 h of culture at 30° C., OpTmizer media supplemented by 10% AB serum and IL-2 was added to the cell suspension, and the mix was incubated for 16 h under the same culture conditions. Cells were subsequently cultivated at 37° C. in the presence of 5% CO₂ and analyzed for TRAC knockout and anti-FAP CAR expression five days later.

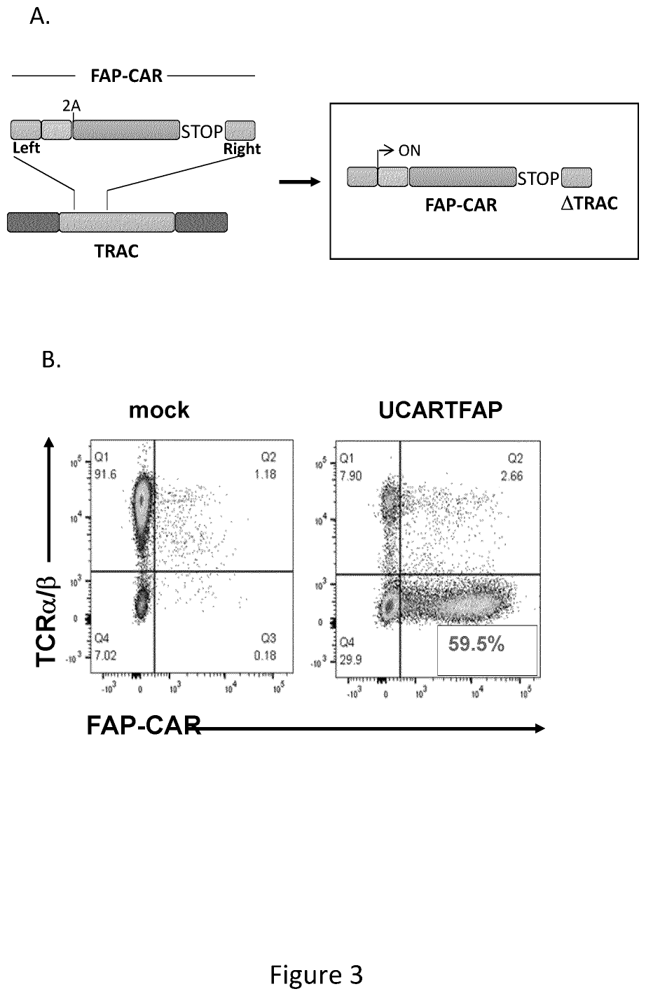

The results showed that more than 59% of engineered UCART-FAP cells were deficient in expression of TCRα/β and expressed the anti-FAP CAR, whereas no CAR expression or gene knockout could be detected in mock transfected T cells (FIG. 3).

### Example 3. Specific Cytolytic Activity of β2M^{KO}UCART-FAP Cells Against Triple-Negative Breast Cancer (TNBC) Patient Derived Cancer-Associated Fibroblasts (CAF)

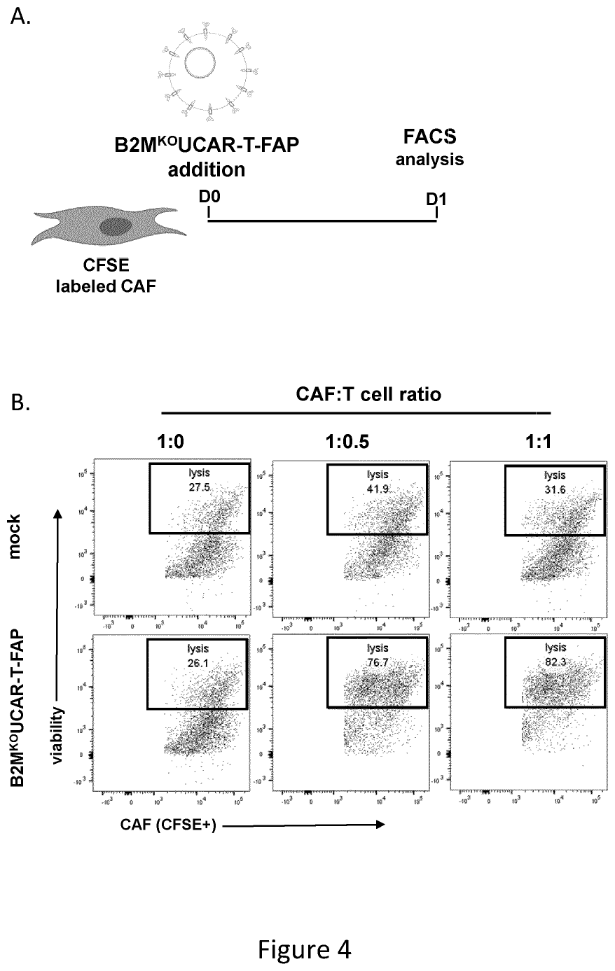

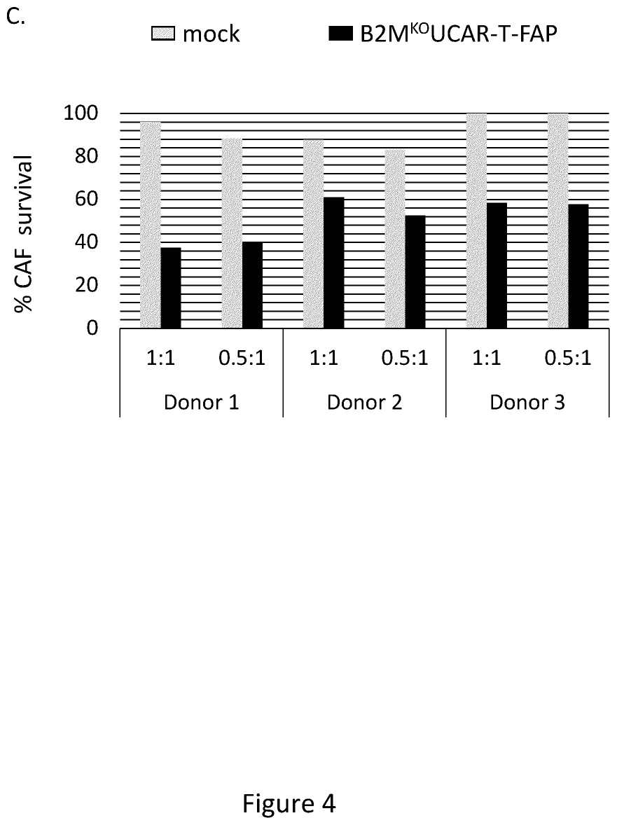

To study the cytolytic activity of the β2M^{KO}UCART-FAP cells engineered in Example 1, relevant CAFs (sourced from BioIVT) were fluorescently labelled with CFSE (0.5 mM) and co-incubated at 37° C., 5% CO₂ with either mock or β2M^{KO}UCART-FAP cells at CAF:T cell ratio of 1:0, 1:0.5 and 1:1 for one day (FIG. 4A). CFSE-labelled CAFs were harvested and stained with a fixable viability dye and analyzed by flow cytometry. CFSE-CAF cells positive for the viability dye were quantitated as dead cells and percentage of CAF lysis was determined for every condition (FIG. 4B).

CAF survival quantification performed on 3 different donors (FIG. 4C) indicates that β2M^{KO}UCART-FAP cells successfully exhibited cytolytic activity against their CAF target cells.

### Example 4. Combinatorial Targeting of Triple-Negative Breast Cancer with β2M^{KO}UCART-FAP and β2M^{KO}UCART-MESO

This example demonstrates the therapeutic advantage of combining β2M^{KO}UCART-FAP treatment with other tumor cell-antigen targeting-UCART, in this example anti-Mesothelin UCART (MESO-UCART) cells.

### Tumor-CAF Spheroid Seeding

To determine whether addition of β2M^{KO}UCART-FAP cells enhances overall anti-tumor cytotoxicity of β2M^{KO}UCART-MESO cells, a 3-dimensional spheroid model of triple negative breast cancer (TNBC) was established. This model allows to mimic the tumor microenvironment, including the spatial organization and properties of an actual tumor. 104 triple-negative breast tumor cells HCC70, transduced to express GFP and reporter gene Nanoluciferase (HCC70-NL-GFP) were seeded either alone or with TNBC-derived CAFs at a 1:1 ratio on low adherence 96-well round bottom plates, in DMEM+10% FBS media. Under these conditions, tumor cells and CAF cells organize themselves into spheroids mimicking in vivo tumor properties.

### β2MN^{KO}UCART-MESO Generation

To express a Mesothelin-CAR (MESO-CAR) on the surface of primary T cells, cryopreserved PBMC were thawed at 37° C., washed and re-suspended in OpTmizer medium supplemented with AB human serum (5%) for overnight incubation at 37° C. in 5% CO₂ incubator. Cells were then activated with Transact in OpTmizer medium supplemented with AB human serum (5%) and recombinant human interleukin-2 (rhIL-2, 350 IU/mL) in a CO₂ incubator (culture medium). Three days after activation, T cells were transduced with lentiviral particle containing a nucleic acid sequence of SEQ ID NO: 128, coding for the anti-MESO CAR of amino acid sequence SEQ ID NO: 106, at an MOI of 15.

Two days after transduction, cells were electroporated with 5 μg of mRNAs encoding TRAC TALEN® arms (SEQ ID NO: 108 and SEQ ID NO: 109) and 5 μg of mRNAs encoding β2M TALEN® arms (SEQ ID NO: 112 and SEQ ID NO: 113). Transfection was performed using Pulse Agile technology by applying two 0.1 mS pulses at 800V followed by four 0.2 mS pulses at 130 V in 0.4 cm gap cuvettes in Cytoporation buffer T (BTX Harvard Apparatus, Holliston, Massachusetts). The electroporated cells were then immediately transferred into prewarmed Optmizer serum-free media and incubated at 37° C. for 15 min and then at 30° C. for 16 h. Cells were thereafter cultivated at 37° C. in the presence of 5% CO₂.

Cytolytic Activity of Combination of B2M^{KO}UCART-MESO Cells and B2M^{KO}UCART-FAP Against Tumor-CAF Spheroids

Cytolytic activity of B2M^{KO}UCART-MESO cells against HCC70-NL-GFP tumor cells in tumor-CAF spheroids was determined by adding B2M^{KO}UCART-MESO to spheroids plated as described above, two days after spheroid seeding, at tumor cell:CAR-T ratio of 1:5. To determine the potential advantage of the combination with B2M^{KO}UCART-FAP cell, B2M^{KO}UCART-FAP were also added to tumor-CAF spheroids at tumor cell:CAR-T ratio of 5:1, either alone or with B2M^{KO}UCART-MESO cells. Mock transfected, non-transduced cells were used as control. 72 h post co-incubation with UCART cells, HCC70-NL-GFP lysis was determined by performing an assay to determine nanoluciferase activity in residual live HCC70-NL-GFP cells, as per manufacturer instructions (Catalog No. N1110, Promega). B2M^{KO}UCART-MESO as well as B2M^{KO}UCART-FAP cells were able to induce around 50% survival of the tumor cells. Most importantly the combination of B2M^{KO}UCART-MESO cells with B2M^{KO}UCART-FAP cells could reduce further the tumor cell survival down to 26%.

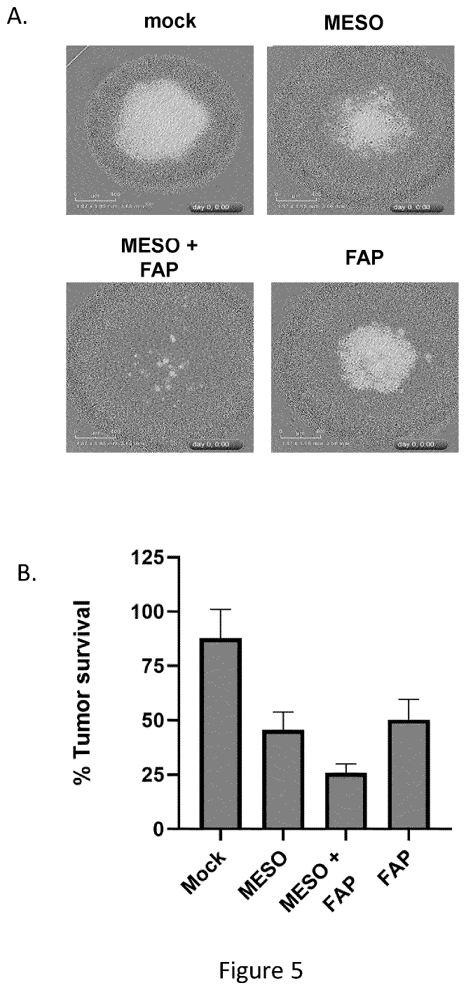

Altogether these results indicate that B2M^{KO}UCART-FAP cells combination with B2M^{KO}UCART-MESO cells significantly enhanced tumor cell lysis and tumor-CAF spheroid regression, relative to B2M^{KO}UCART-MESO treatment alone (FIG. 5).

These results reveal that B2M^{KO}UCART-FAP is able to turn a “cold” tumor (i.e. resisting to T cell killing) into a “hot” tumor (i.e. prone to T cell killing). Therefore, B2M^{KO}UCART-FAP has a potential to be combined with other immunotherapy treatment for eliciting a stronger immune response.

### Example 5. Combination of CAR-T-FAP and Anti-PD-1 Antibody in Mouse Model

This example demonstrates the therapeutic advantage of combining CART-FAP treatment with anti-PD-1 checkpoint inhibitor for treating breast cancer in vivo.

### Generating Murine Model of Breast Cancer

To assess the therapeutic advantage of combining CART-FAP treatment with anti-PD-1 checkpoint inhibitor in vivo, a tumor model with an intact immune system is needed in order to assess TIL levels and checkpoint inhibition impact. A murine breast tumor model with mouse CART-FAP cells as surrogates was generated for proof-of-concept.

0.5×10⁶ 4-T1 mouse breast cancer cells (ATCC) were orthotopically implanted in the left inguinal mammary fat pad of 8 weeks old, female, immune competent BALB/cJ mice. The resulting mammary tumor established closely recapitulates the physiology of human breast tumors, with cancer-associated fibroblasts in the tumor microenvironment and poor T cell infiltration (Liao D et al. 2009, PLOS One, 4:11). It is therefore a suitable model to study the cytotoxic activity of CART-FAP cells on CAF and the subsequent effect on T cell tumor infiltration. It also demonstrates the potential advantage of combining CART-FAP and anti-PD-1 therapies.

### Mouse CART-FAP Cell Generation

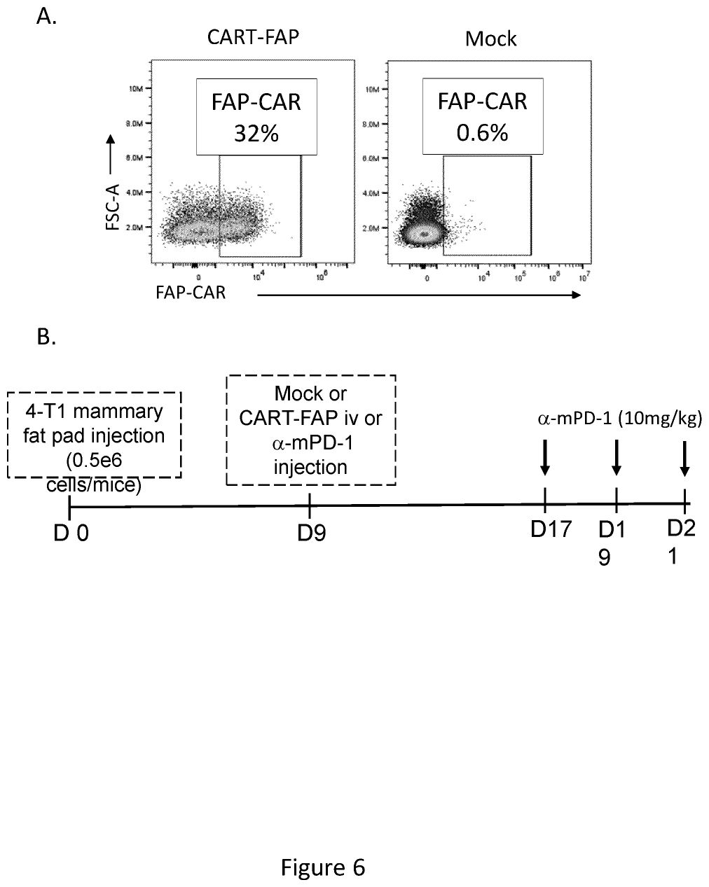

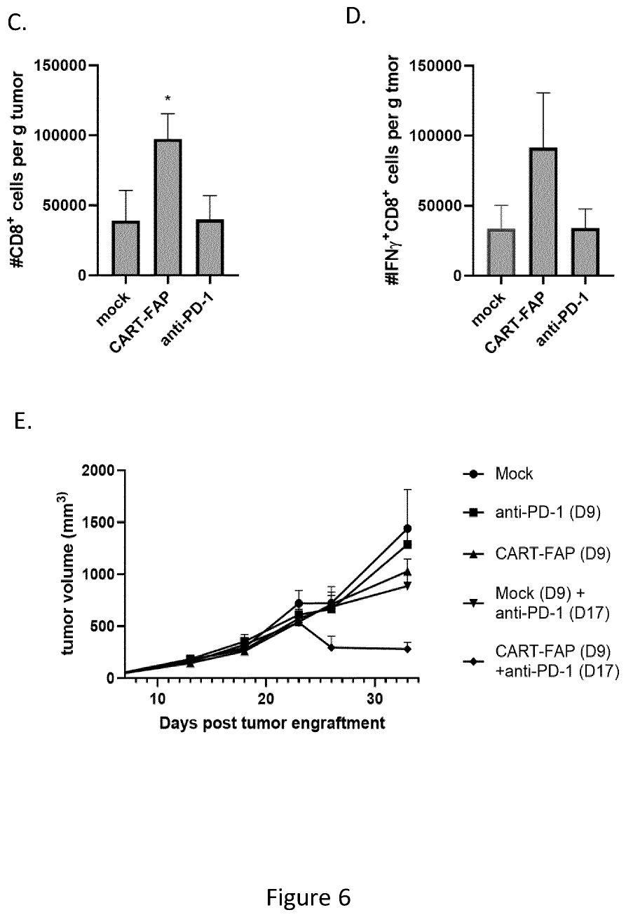

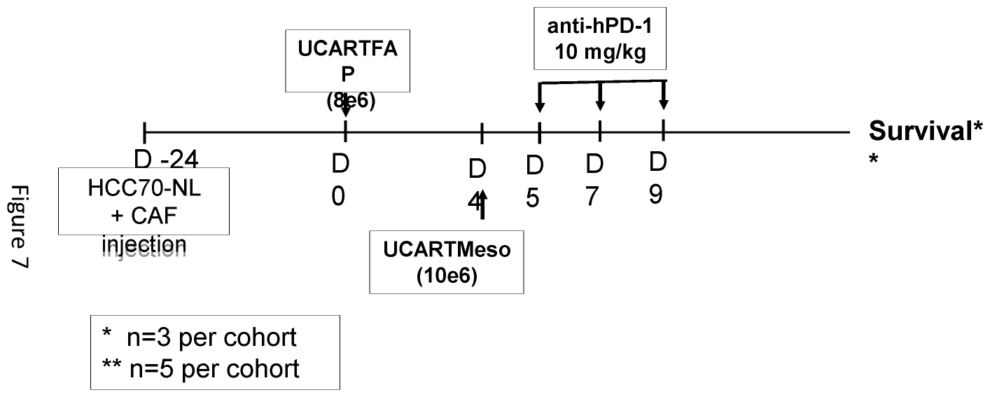

Primary murine splenic T cells were isolated using the “EasySep mouse T cell isolation kit” (StemCell Technologies) and activated with Dynabeads mouse T cell activator (Gibco) with 100 U/ml IL-2 overnight. After 24 hours, cells (1×10⁶ cells/well) were transduced with lentiviral particles containing a nucleotide sequence encoding the CLSFAP3-CAR of amino acid sequence SEQ ID NO: 31, directed against human and murine FAP, at an MOI of 25 in a 24-well plate coated with Retronectin (50 ug/mL; Takara), and centrifuged at room temperature for 45 minutes at 1200 g. After overnight incubation, cells were expanded with 100 U/ml of IL-2 for additional 14 days at 37° C. in the presence of 5% CO₂, with re-activation using Dynabeads at Day 7. Cells were analysed by flow cytometry. The results show that around 30% of the T cells expressed the CLSFAP3-CAR after expansion (FIG. 6A).

### Anti-Tumor Activity of Combination of Murine CART-FAP Cells and Anti-PD-1 Checkpoint Inhibitor in Orthotopic Breast Tumor Mouse Model.

To assess the anti-tumor efficacy of CART-FAP in combination with an anti-PD-1 checkpoint inhibitor, 4-T1 breast tumor bearing mice modelled as described above were treated once the tumors reached a volume of ˜50 mm³ with either 10×10⁶ murine CART-FAP cells or with an anti-mouse PD-1 antibody (In VivoMab anti-mouse PD-1 (CD279), Clone: RMP1-14, BioXCell) (FIG. 6B). Eight days later (D17), a subset of these mice was euthanised, tumors were excised and processed to single-cell suspension by digestion with Accutase at 37° C. for 15 minutes. Flow cytometry analysis of the tumor cell suspension revealed significantly increased infiltration (FIG. 6C) and activation (FIG. 6D) of CD8 T cells in CART-FAP treated tumors, relative to mock or anti-PD-1 treated cohorts. The remaining mice subset was further treated with an anti-mouse PD-1 (BioXCell, as outlined in FIG. 6B). This additional anti-PD1 treatment resulted in significant regression of the primary breast tumor compared to the mice cohorts which have only been pre-treated with CART-FAP (FIG. 6E).

These results, thus, demonstrate the ability of CART-FAP to promote CD8 T cell infiltration in tumors and further synergise with anti-PD-1 checkpoint blockade for enhanced tumor regression.

### Example 6. Combinatorial Targeting of Triple-Negative Breast Cancer with B2M^{KO}UCART-FAP Cells, CD52^{KO}TGFbR2^{KO}UCART-MESO Cells and Anti-PD1 Monoclonal Antibody

This example demonstrates the therapeutic advantage of combining B2M^{KO}UCART-FAP treatment with CD52^{KO}TGFbR2^{KO}UCART-MESO and anti-PD-1 checkpoint inhibitor for treating triple-negative breast cancer in vivo.

### Human UCART Cell Generation

B2M^{KO}UCART-FAP cells were generated as described in Example 1. The generation of CD52^{KO}TGFbR2^{KO}UCART-MESO was performed as followed. Cryopreserved PBMC were thawed at 37° C., washed and re-suspended in OpTmizer medium supplemented with AB human serum (5%) for overnight incubation at 37° C. in 5% CO₂ incubator. Cells were then activated with Transact in OpTmizer medium supplemented with AB human serum (5%) and recombinant human interleukin-2 (rhIL-2, 350 IU/mL) in a CO₂ incubator (culture medium). Three days after activation, T cells were transduced with lentiviral particle containing a nucleotide sequence encoding an anti-MESO CAR of SEQ ID NO: 106 at an MOI of 15. The nucleotide sequence used in this example was SEQ ID NO: 128.

### Knockout of TRAC, CD52 and TGFBRII

Three days after transduction, anti-MESO-CAR-T cells were electroporated with 0.25 ug of mRNAs encoding TRAC TALEN® arms (SEQ ID NO: 108 and SEQ ID NO: 109) and 0.25 ug of mRNAs encoding CD52 TALEN® arms (SEQ ID NO: 110 and SEQ ID NO: 111) per million cells. Transfection was performed using Pulse Agile technology by applying two 0.1 mS pulses at 800 V followed by four 0.2 mS pulses at 130 V in 0.4 cm gap cuvettes in Cytoporation buffer T (BTX Harvard Apparatus, Holliston, Massachusetts). The electroporated cells were then immediately transferred into prewarmed Optmizer serum-free media and incubated at 37° C. for 15 min and then at 30° C. for 16 h. Cells were thereafter cultivated at 37° C. in the presence of 5% CO₂.

Three days after electroporation, CD52^{KO}UCART-MESO were electroporated with mRNAs encoding TGFBRII TALEN® arms (SEQ ID NO: 141 and SEQ ID NO: 142). Transfection was performed using Pulse Agile technology by applying two 0.1 mS pulses at 800 V followed by four 0.2 mS pulses at 130 V in 0.4 cm gap cuvettes in Cytoporation buffer T (BTX Harvard Apparatus, Holliston, Massachusetts). The electroporated cells were then immediately transferred into prewarmed Optmizer serum-free media and incubated at 37° C. for 15 min and then at 30° C. for 16 h. Cells were thereafter cultivated at 37° C. in the presence of 5% CO₂.

Anti-Tumor Activity of Combination of B2M^{KO}UCART-FAP Cells, CD52^{KO}TGFbR2^{KO}UCART-MESO Cells and Anti-PD1 mAb in an In Vivo Mouse Model.

To determine the therapeutic advantage of combining B2M^{KO}UCART-FAP treatment with CD52^{KO}TGFbR2^{KO}UCART-MESO and anti-PD-1 checkpoint inhibitor in vivo, 8-week-old, female NSG mice were orthotopically implanted with 3×10⁶ human triple-negative breast cancer cell line HCC70-NanoLuc-GFP mixed with 3×10⁶ human triple-negative breast tumor derived cancer-associated fibroblasts in the left inguinal mammary fat pad. 24 days post tumor implantation, tumor-bearing mice were intravenously injected with 8×10⁶ mock transfected or B2M^{KO}UCART-FAP cells. Four days later, these mice were i.v. injected with 10×10⁶ CD52^{KO}TGFbR2^{KO}UCART-MESO cells. Anti-hPD-1 (BioXCell, Catalog No. SIM0003) treatment was initiated the following day, as indicated in FIG. 7.

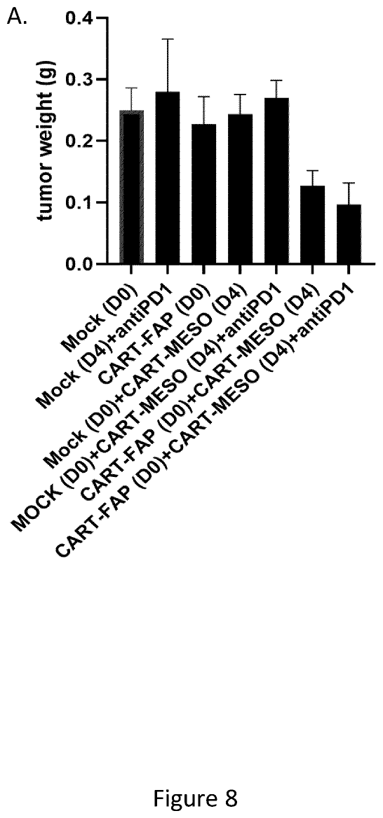

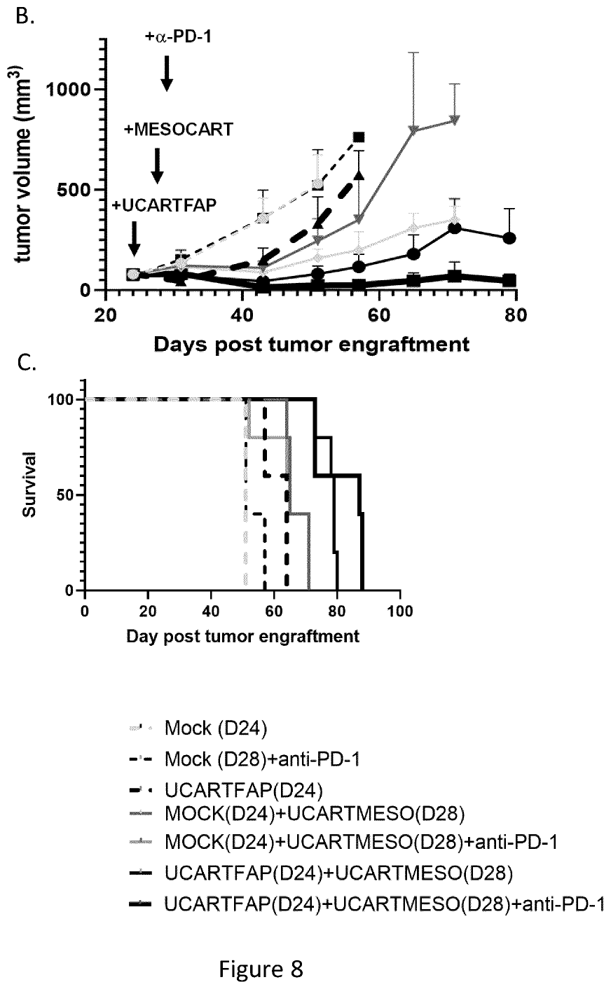

Anti-PD-1 treatment of tumor-bearing mice alone or in combination with CD52^{KO}TGFbR2^{KO}UCART-MESO cells did not affect tumor progression (FIG. 8). Tumors were also unresponsive to single treatment with CD52^{KO}TGFbR2^{KO}UCART-MESO cells. Combination of B2M^{KO}UCART-FAP treatment with CD52^{KO}TGFbR2^{KO}UCART-MESO resulted in significant tumor regression, indicating the advantage of B2M^{KO}UCART-FAP-mediated depletion of CAFs in potentiating CD52^{KO}TGFbR2^{KO}UCART-MESO anti-tumor activity. Furthermore, B2M^{KO}UCART-FAP pre-treatment, followed by anti-PD-1 and CD52^{KO}TGFbR2^{KO}UCART-MESO combination treatment led to the highest level of tumor regression and significantly enhanced mouse survival. Our results thus demonstrate the critical role of B2M^{KO}UCART-FAP-mediated tumor microenvironment reprogramming to enhance infiltration of CD52^{KO}TGFbR2^{KO}UCART-MESO and induce positive response to anti-PD-1 checkpoint blockade.

1-47. (canceled)

48. An allogeneic treatment method of treating a solid tumor in a patient in need thereof, comprising
lymphodepleting the patient's immune cells, and
administering to the patient (i) an effective amount of engineered TCR-negative immune cells expressing at their cell surface a Chimeric Antigen Receptor (CAR) directed against Fibroblast Activation Protein (FAP), and (ii) an effective amount of at least one immune checkpoint antagonist,
wherein the engineered TCR-negative immune cells are engineered T-cells comprising an inactivated TCR or (ii) Natural Killer (NK) cells, and
wherein the engineered TCR-negative immune cells originate from a human who is not the patient.

49. The method of claim 48, wherein the CAR directed against Fibroblast Activation Protein (FAP-CAR), comprises:
(a) an extracellular ligand binding-domain comprising VH and VL amino acid sequences from a monoclonal anti-FAP antibody,
(b) a hinge amino acid sequence selected from a FcγRIII hinge, a CD8α hinge and an IgG1 hinge,
(c) a transmembrane domain amino acid sequence comprising a CD8α transmembrane domain or a CD28 transmembrane domain, and
(d) a cytoplasmic domain comprising amino acid sequences from a CD3 zeta signaling domain and a co-stimulatory domain from 4-1BB or from CD28.

50. The method of claim 48, wherein the CAR directed against Fibroblast Activation Protein comprises an extracellular binding-domain comprising:
the H-CDRs of SEQ ID NO: 1, SEQ ID NO: 2, and SEQ ID NO: 3, and the L-CDRs of SEQ ID NO: 4, SEQ ID NO: 5, and SEQ ID NO: 6, and at least 80% identity with VH of amino acid sequence SEQ ID NO: 7 and VL of amino acid sequence SEQ ID NO: 8;
the H-CDRs of SEQ ID NO: 12, SEQ ID NO: 13, and SEQ ID NO: 14, and the L-CDRs of SEQ ID NO: 15, SEQ ID NO: 16, and SEQ ID NO: 17, and at least 80% identity with VH of amino acid sequence SEQ ID NO: 18 and VL of amino acid sequence SEQ ID NO: 19;
the H-CDRs of SEQ ID NO: 23, SEQ ID NO: 24, and SEQ ID NO: 25, and the L-CDRs of SEQ ID NO: 26, SEQ ID NO: 27, and SEQ ID NO: 28, and at least 80% identity with VH of amino acid sequence SEQ ID NO: 29 and VL of amino acid sequence SEQ ID NO: 30; and/or
the H-CDRs of SEQ ID NO: 34, SEQ ID NO: 35, and SEQ ID NO: 36, and the L-CDRs of SEQ ID NO: 37, SEQ ID NO: 38, and SEQ ID NO: 39, and at least 80% identity with VH of amino acid sequence SEQ ID NO: 40 and VL of amino acid sequence SEQ ID NO: 41.

51. The method of claim 48, wherein the CAR directed against Fibroblast Activation Protein comprises an extracellular binding-domain comprising the amino acid sequence SEQ ID NO: 9, SEQ ID NO: 20, SEQ ID NO: 31, or SEQ ID NO: 42.

52. The method of claim 48, further comprising administering (iii) an effective amount of engineered TCR-negative immune cells expressing at their cell surface a CAR binding an antigen associated with a cancer selected from Mesothelin, Trop2, MUC1, EGFR, and VEGF, wherein the engineered immune cells originate from a human who is not the patient.

53. The method of claim 48, wherein the engineered T-cells have been genetically modified to suppress or repress expression of at least one MHC protein selected from β2m and HLA, in the T-cells.

54. The method of claim 48, wherein the engineered T-cells have been genetically modified to suppress or repress expression of an immune checkpoint protein and/or the receptor thereof, in the T-cells.

55. The method of claim 48, wherein the engineered T-cells have been genetically modified to confer resistance to at least one immune suppressive or chemotherapy drug, and optionally to comprise a suicide gene.

56. The method of claim 48, wherein the engineered T-cells derive from inflammatory T-lymphocytes, cytotoxic T-lymphocytes, or helper T-lymphocytes.

57. The method of claim 48, wherein the immune checkpoint antagonist is an antibody directed against an immune checkpoint protein and/or a receptor thereof, wherein the immune checkpoint protein or receptor thereof is selected from the group consisting of PD1, PDL1, CTLA4, LAG3, TIM3, TIGIT, VISTA, GITR and BTLA.

58. The method of claim 57, wherein the immune checkpoint antagonist is an anti-PD1 antibody or an anti-PDL1 antibody.

59. The method of claim 58, wherein the immune checkpoint antagonist is an anti-PD1 antibody selected from the group consisting of pembrolizumab, nivolumab, cemiplimab, and spartalizumab, or an anti-PDL1 antibody selected from the group consisting of durvalumab, atezolizumab and avelumab.

60. The method of claim 48, wherein administration of the engineered T-cells and the administration of the immune checkpoint antagonist are carried out sequentially.

61. The method of claim 60, wherein the administration of said immune checkpoint antagonist starts between 1 week and 3 months after administration of the engineered TCR-negative immune cells of (i).

62. A pharmaceutical composition comprising (i) engineered T-cells comprising an inactivated TCR and expressing at their cell surface a Chimeric Antigen Receptor (CAR) directed against Fibroblast Activation Protein (FAP) (UCART-FAP), and (ii) an immune checkpoint antagonist, wherein both components (i) and (ii) are formulated for separate allogeneic administration, in a patient from whom the engineered T-cells do not originate.

63. The pharmaceutical composition of claim 62, further comprising (iii) engineered TCR-negative immune cells expressing at their cell surface a CAR binding an antigen associated with a cancer selected from Mesothelin, Trop2, MUC1, EGFR, and VEGF, wherein components (i), (ii) and (iii) are formulated for separate allogeneic administration, in a patient from whom the engineered T-cells do not originate.


SEQUENCE LISTING (142 sequences)

SEQ ID NO: 1 - protein, 9 aa, artificial
  H-CDR1
YTFTEYTIH

SEQ ID NO: 2 - protein, 15 aa, artificial
  H-CDR2
GINPNNGIPNYNQKF

SEQ ID NO: 3 - protein, 15 aa, artificial
  H-CDR3
RRIAYGYDEGHAMDY

SEQ ID NO: 4 - protein, 14 aa, artificial
  L-CDR1
QSLLYSRNQKNYLA

SEQ ID NO: 5 - protein, 11 aa, artificial
  L-CDR2
LLIFWASTRES

SEQ ID NO: 6 - protein, 9 aa, artificial
  L-CDR3
QQYFSYPLT

SEQ ID NO: 7 - protein, 124 aa, artificial
  VH
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 8 - protein, 112 aa, artificial
  VL
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEI

SEQ ID NO: 9 - protein, 253 aa, artificial
  scFv
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGSTSGSGKPGSGEGSTKDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEI

SEQ ID NO: 10 - protein, 511 aa, artificial
  FAP1-41BB-CAR
MALPVTALLLPLALLLHAARPQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGSTSGSGKPGSGEGSTKDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 499 aa, artificial
  FAP1-CD28-CAR
MALPVTALLLPLALLLHAARPQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGSTSGSGKPGSGEGSTKDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEITTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 9 aa, artificial
  H-CDR1
YTFTDYYMK

SEQ ID NO: 13 - protein, 15 aa, artificial
  H-CDR2
DIYPNNGEIPYNQKF

SEQ ID NO: 14 - protein, 12 aa, artificial
  H-CDR3
VRGYYYGLAMDY

SEQ ID NO: 15 - protein, 11 aa, artificial
  L-CDR1
TGAVTTSNYAN

SEQ ID NO: 16 - protein, 11 aa, artificial
  L-CDR2
GLIGATNNRAP

SEQ ID NO: 17 - protein, 9 aa, artificial
  L-CDR3
ALWYSNHFI

SEQ ID NO: 18 - protein, 119 aa, artificial
  VH
EVQLQQSGPELVKPGASVRMSCKASGYTFTDYYMKWVKQSLGKSLEWIGDIYPNNGEIPYNQKFKGKATLTADKTSSTAYMQLNSLTSEDSAVYYCVRGYYYGLAMDYWGQGTSVTSVV

SEQ ID NO: 19 - protein, 108 aa, artificial
  VL
QAVVTQESALTSPGETVTLTCRSSTGAVTTSNYANWVQEKPDRLFTGLIGATNNRAPGVPARFSGSLIGDKAALTITGAQTEDEAIYFCALWYSNHFIFGSGTKVTVL

SEQ ID NO: 20 - protein, 242 aa, artificial
  scFv
EVQLQQSGPELVKPGASVRMSCKASGYTFTDYYMKWVKQSLGKSLEWIGDIYPNNGEIPYNQKFKGKATLTADKTSSTAYMQLNSLTSEDSAVYYCVRGYYYGLAMDYWGQGTSVTSVVGGGGSGGGGSGGGGSQAVVTQESALTSPGETVTLTCRSSTGAVTTSNYANWVQEKPDRLFTGLIGATNNRAPGVPARFSGSLIGDKAALTITGAQTEDEAIYFCALWYSNHFIFGSGTKVTVL

SEQ ID NO: 21 - protein, 515 aa, artificial
  FAP2-41BB-CAR
MALPVTALLLPLALLLHAARPEVQLQQSGPELVKPGASVRMSCKASGYTFTDYYMKWVKQSLGKSLEWIGDIYPNNGEIPYNQKFKGKATLTADKTSSTAYMQLNSLTSEDSAVYYCVRGYYYGLAMDYWGQGTSVTSVVGGGGSGGGGSGGGGSQAVVTQESALTSPGETVTLTCRSSTGAVTTSNYANWVQEKPDRLFTGLIGATNNRAPGVPARFSGSLIGDKAALTITGAQTEDEAIYFCALWYSNHFIFGSGTKVTVLGGGGSGGGGSGGGGSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 495 aa, artificial
  FAP2-CD28-CAR
MALPVTALLLPLALLLHAARPEVQLQQSGPELVKPGASVRMSCKASGYTFTDYYMKWVKQSLGKSLEWIGDIYPNNGEIPYNQKFKGKATLTADKTSSTAYMQLNSLTSEDSAVYYCVRGYYYGLAMDYWGQGTSVTSVVGGGGSGGGGSGGGGSQAVVTQESALTSPGETVTLTCRSSTGAVTTSNYANWVQEKPDRLFTGLIGATNNRAPGVPARFSGSLIGDKAALTITGAQTEDEAIYFCALWYSNHFIFGGGGGSGGGGSGGGGSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 9 aa, artificial
  H-CDR1
YTFTENIIH

SEQ ID NO: 24 - protein, 15 aa, artificial
  H-CDR2
WFHPGSGSIKYNEKF

SEQ ID NO: 25 - protein, 11 aa, artificial
  H-CDR3
HGGTGRGAMDY

SEQ ID NO: 26 - protein, 12 aa, artificial
  L-CDR1
KSVSTSAYSYMH

SEQ ID NO: 27 - protein, 11 aa, artificial
  L-CDR2
LLIYLASNLES

SEQ ID NO: 28 - protein, 9 aa, artificial
  L-CDR3
QHSRELPYT

SEQ ID NO: 29 - protein, 120 aa, artificial
  VH
QVQLVQSGAEVKKPGASVKVSCKASGYTFTENIIHWVRQAPGQGLEWMGWFHPGSGSIKYNEKFKDRVTMTADTSTSTVYMELSSLRSEDTAVYYCARHGGTGRGAMDYWGQGTLVTVSS

SEQ ID NO: 30 - protein, 112 aa, artificial
  VL
DIQMTQSPSSLSASVGDRVTITCRASKSVSTSAYSYMHWYQQKPGKAPKLLIYLASNLESGVPSRFSGSGSGTDFTLTISSLQPEDFATYYCQHSRELPYTFGQGTKLEIKR

SEQ ID NO: 31 - protein, 247 aa, artificial
  scFv
QVQLVQSGAEVKKPGASVKVSCKASGYTFTENIIHWVRQAPGQGLEWMGWFHPGSGSIKYNEKFKDRVTMTADTSTSTVYMELSSLRSEDTAVYYCARHGGTGRGAMDYWGQGTLVTVSSGGGGSGGGGSGGGGSDIQMTQSPSSLSASVGDRVTITCRASKSVSTSAYSYMHWYQQKPGKAPKLLIYLASNLESGVPSRFSGSGSGTDFTLTISSLQPEDFATYYCQHSRELPYTFGQGTKLEIKR

SEQ ID NO: 32 - protein, 520 aa, artificial
  FAP3-41BB-CAR
MALPVTALLLPLALLLHAARPQVQLVQSGAEVKKPGASVKVSCKASGYTFTENIIHWVRQAPGQGLEWMGWFHPGSGSIKYNEKFKDRVTMTADTSTSTVYMELSSLRSEDTAVYYCARHGGTGRGAMDYWGQGTLVTVSSGGGGSGGGGSGGGGSDIQMTQSPSSLSASVGDRVTITCRASKSVSTSAYSYMHWYQQKPGKAPKLLIYLASNLESGVPSRFSGSGSGTDFTLTISSLQPEDFATYYCQHSRELPYTFGQGTKLEIKRGGGGSGGGGSGGGGSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 508 aa, artificial
  FAP3-CD28-CAR
MALPVTALLLPLALLLHAARPQVQLVQSGAEVKKPGASVKVSCKASGYTFTENIIHWVRQAPGQGLEWMGWFHPGSGSIKYNEKFKDRVTMTADTSTSTVYMELSSLRSEDTAVYYCARHGGTGRGAMDYWGQGTLVTVSSGGGGSGGGGSGGGGSDIQMTQSPSSLSASVGDRVTITCRASKSVSTSAYSYMHWYQQKPGKAPKLLIYLASNLESGVPSRFSGSGSGTDFTLTISSLQPEDFATYYCQHSRELPYTFGQGTKLEIKRGGGGSGGGGSGGGGSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 9 aa, artificial
  H-CDR1
FTFSSYAMS

SEQ ID NO: 35 - protein, 17 aa, artificial
  H-CDR2
VSAIIGSGASTYYADSV

SEQ ID NO: 36 - protein, 9 aa, artificial
  H-CDR3
KGWFGGFNY

SEQ ID NO: 37 - protein, 9 aa, artificial
  L-CDR1
QSVTSSYLA

SEQ ID NO: 38 - protein, 13 aa, artificial
  L-CDR2
LLINVGSRRATGI

SEQ ID NO: 39 - protein, 9 aa, artificial
  L-CDR3
QQGIMLPPT

SEQ ID NO: 40 - protein, 117 aa, artificial
  VH
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 41 - protein, 108 aa, artificial
  VL
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 42 - protein, 242 aa, artificial
  scFv
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSGSTSGSGKPGSGEGSTKEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 43 - protein, 513 aa, artificial
  FAP4-41BB-CAR
MALPVTALLLPLALLLHAARPEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSGGGGSGGGGSGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 488 aa, artificial
  FAP4-CD28-CAR
MALPVTALLLPLALLLHAARPEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSGSTSGSGKPGSGEGSTKEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 17 aa, artificial
  Linker1
GSTSGSGKPGSGEGSTK

SEQ ID NO: 46 - protein, 15 aa, artificial
  Linker2
GGGGSGGGGSGGGGS

SEQ ID NO: 47 - protein, 9 aa, artificial
  CD3 H-CDR1
YTFTRYTMH

SEQ ID NO: 48 - protein, 20 aa, artificial
  CD3 H-CDR2
WIGYINPSRGYTNYNQKFKD

SEQ ID NO: 49 - protein, 10 aa, artificial
  CD3 H-CDR3
YYDDHYCLDY

SEQ ID NO: 50 - protein, 14 aa, artificial
  CD3 L-CDR1
TMTCRASSSVSYMN

SEQ ID NO: 51 - protein, 11 aa, artificial
  CD3 L-CDR2
RWIYDTSKVAS

SEQ ID NO: 52 - protein, 9 aa, artificial
  CD3 L-CDR3
QQWSSNPLT

SEQ ID NO: 53 - protein, 243 aa, artificial
  CD3 ScFv of BiTE
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 54 - protein, 9 aa, artificial
  CD3a H-CDR1
FTFSGYGMH

SEQ ID NO: 55 - protein, 18 aa, artificial
  CD3a H-CDR2
SVAYITSSSINIKYADAV

SEQ ID NO: 56 - protein, 7 aa, artificial
  CD3a H-CDR3
FDWDKNY

SEQ ID NO: 57 - protein, 8 aa, artificial
  CD3a L-CDR1
QDISNYLN

SEQ ID NO: 58 - protein, 11 aa, artificial
  CD3a L-CDR2
LLIYYTNKLAD

SEQ ID NO: 59 - protein, 9 aa, artificial
  CD3a L-CDR3
QQYYNYPWT

SEQ ID NO: 60 - protein, 239 aa, artificial
  CD3a ScFv of BiTE
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKR

SEQ ID NO: 61 - protein, 9 aa, artificial
  CD16 H-CDR1
FTFDDYGMS

SEQ ID NO: 62 - protein, 18 aa, artificial
  CD16 H-CDR2
WVSGINWNGGSTGYADSV

SEQ ID NO: 63 - protein, 8 aa, artificial
  CD16 H-CDR3
GRSLLFDY

SEQ ID NO: 64 - protein, 11 aa, artificial
  CD16 L-CDR1
QGDSLRSYYAS

SEQ ID NO: 65 - protein, 11 aa, artificial
  CD16 L-CDR2
LVIYGKNNRPS

SEQ ID NO: 66 - protein, 9 aa, artificial
  CD16 L-CDR3
NSRDSSGNH

SEQ ID NO: 67 - protein, 240 aa, artificial
  CD16 ScFv of BiTE
MEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 68 - protein, 9 aa, artificial
  Trop2 H-CDR1
YTFTNYGMN

SEQ ID NO: 69 - protein, 14 aa, artificial
  Trop2 H-CDR2
MGWINTYTGEPTYT

SEQ ID NO: 70 - protein, 12 aa, artificial
  Trop2 H-CDR3
GGFGSSYWYFDV

SEQ ID NO: 71 - protein, 8 aa, artificial
  Trop2 L-CDR1
QDVSIAVA

SEQ ID NO: 72 - protein, 11 aa, artificial
  Trop2 L-CDR2
LLIYSASYRYT

SEQ ID NO: 73 - protein, 9 aa, artificial
  Trop2 L-CDR3
QQHYITPLT

SEQ ID NO: 74 - protein, 243 aa, artificial
  Trop2 ScFv of BiTE
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 75 - protein, 5 aa, artificial
  MUC1 H-CDR1
NYGLS

SEQ ID NO: 76 - protein, 16 aa, artificial
  MUC1 H-CDR2
ENHPGSGIIYHNEKFR

SEQ ID NO: 77 - protein, 9 aa, artificial
  MUC1 H-CDR3
SSGTRGFAY

SEQ ID NO: 78 - protein, 16 aa, artificial
  MUC1 L-CDR1
RSSQSIVHSNGNTYLE

SEQ ID NO: 79 - protein, 11 aa, artificial
  MUC1 L-CDR2
LLIYKVSNRFS

SEQ ID NO: 80 - protein, 9 aa, artificial
  MUC1 L-CDR3
FQGSHGPWT

SEQ ID NO: 81 - protein, 285 aa, artificial
  MUC1 ScFv of BiTE
MEWIWIFLFILSGTAGVQSQVQLQQSGAELARPGASVKLSCKASGYTFTNYGLSWVKQRTGQGLEWIGENHPGSGIIYHNEKFRGKATLTADKSSSTAYVQLSSLTSEDSAVYFCARSSGTRGFAYWGQGTLVTVSAGGGGSGGGGSGGGGSMKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHGPWTFGGGTKLEIKRA

SEQ ID NO: 82 - protein, 9 aa, artificial
  Meso H-CDR1
INNNNYYWT

SEQ ID NO: 83 - protein, 19 aa, artificial
  Meso H-CDR2
WIGYIYYSGSTFYNPSLKS

SEQ ID NO: 84 - protein, 9 aa, artificial
  Meso H-CDR3
EDTMTGLDV

SEQ ID NO: 85 - protein, 8 aa, artificial
  Meso L-CDR1
QSINNYLN

SEQ ID NO: 86 - protein, 11 aa, artificial
  Meso L-CDR2
LLIYAASSLQS

SEQ ID NO: 87 - protein, 8 aa, artificial
  Meso L-CDR3
QQTYSNPT

SEQ ID NO: 88 - protein, 240 aa, artificial
  MESO ScFv of BiTE
QVQLQESGPGLVKPSQTLSLTCTVSGGSINNNNYYWTWIRQHPGKGLEWIGYIYYSGSTFYNPSLKSRVTISVDTSKTQFSLKLSSVTAADTAVYYCAREDTMTGLDVWGQGTTVTVSSGGGGSGGGGSGGGGSDIQMTQSPSSLSASVGDRVTITCRASQSINNYLNWYQQKPGKAPTLLIYAASSLQSGVPSRFSGSRSGTDFTLTISSLQPEDFAAYFCQQTYSNPTFGQGTKVEVK

SEQ ID NO: 89 - protein, 9 aa, artificial
  CD40 H-CDR1
FTFSDYYMY

SEQ ID NO: 90 - protein, 18 aa, artificial
  CD40 H-CDR2
WVAYINSGGGSTYYPDTV

SEQ ID NO: 91 - protein, 10 aa, artificial
  CD40 H-CDR3
RGLPFHAMDY

SEQ ID NO: 92 - protein, 8 aa, artificial
  CD40 L-CDR1
QGISNYLN

SEQ ID NO: 93 - protein, 11 aa, artificial
  CD40 L-CDR2
LLIYYTSILHS

SEQ ID NO: 94 - protein, 9 aa, artificial
  CD40 L-CDR3
QQFNKLPPT

SEQ ID NO: 95 - protein, 242 aa, artificial
  CD40 ScFv1 of BiTE
EVKLVESGGGLVQPGGSLKLSCATSGFTFSDYYMYWVRQTPEKRLEWVAYINSGGGSTYYPDTVKGRFTISRDNAKNTLYLQMSRLKSEDTAMYYCARRGLPFHAMDYWGQGTSVTVSGSTSGSGKPGSGEGSTKDIQMTQTTSSLSASLGDRVTISCSASQGISNYLNWYQQKPDGTVKLLIYYTSILHSGVPSRFSGSGSGTDYSLTIGNLEPEDIATYYCQQFNKLPPTFGGGTKLEIK

SEQ ID NO: 96 - protein, 9 aa, artificial
  CD40-2 H-CDR1
YTFTSYWMH

SEQ ID NO: 97 - protein, 19 aa, artificial
  CD40-2 H-CDR2
IGNIDPSNGETHYNQKFDR

SEQ ID NO: 98 - protein, 16 aa, artificial
  CD40-2 H-CDR3
ERIYYSGSTYDGYFDV

SEQ ID NO: 99 - protein, 7 aa, artificial
  CD40-2 L-CDR1
SSLSYMH

SEQ ID NO: 100 - protein, 11 aa, artificial
  CD40-2 L-CDR2
RWIYDTSKLAS

SEQ ID NO: 101 - protein, 9 aa, artificial
  CD40-2 L-CDR3
QQWSSNPWT

SEQ ID NO: 102 - protein, 249 aa, artificial
  CD40 ScFv2 of BiTE
EVQLVQSGAEGVKKPGSSVKVSCKASGYTFTSYWMHWVRQAPGQGLEWIGNIDPSNGETHYNQKFDRATLTVDKSTSTAYMEGLSSLRSEDTAVYYCARERIYYSGSTYDGYFDVWGQGTTVTVSSGSTSGSGKPGSGEGSTKDIQLTQSPSPLSASVGDRVTITCSASSSLSYMHWYQQKPGKSPKRWIYDTSKLASGVPSRFSGSGSGTEYTLTISSLQPEDFATYYCQQWSSNPWTFGGGTKVEIK

SEQ ID NO: 103 - protein, 491 aa, artificial
  TROP2-CD3 BiTE
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 104 - protein, 484 aa, artificial
  Mesothelin-CD3 BiTE
QVQLQESGPGLVKPSQTLSLTCTVSGGSINNNNYYWTWIRQHPGKGLEWIGYIYYSGSTFYNPSLKSRVTISVDTSKTQFSLKLSSVTAADTAVYYCAREDTMTGLDVWGQGTTVTVSSGGGGSGGGGSGGGGSDIQMTQSPSSLSASVGDRVTITCRASQSINNYLNWYQQKPGKAPTLLIYAASSLQSGVPSRFSGSRSGTDFTLTISSLQPEDFAAYFCQQTYSNPTFGQGTKVEVKGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKR

SEQ ID NO: 105 - protein, 524 aa, artificial
  MUC1-CD3 BiTE
MEWIWIFLFILSGTAGVQSQVQLQQSGAELARPGASVKLSCKASGYTFTNYGLSWVKQRTGQGLEWIGENHPGSGIIYHNEKFRGKATLTADKSSSTAYVQLSSLTSEDSAVYFCARSSGTRGFAYWGQGTLVTVSAGGGGSGGGGSGGGGSMKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHGPWTFGGGTKLEIKRAEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKR

SEQ ID NO: 106 - protein, 528 aa, artificial
  MESO-CAR
MALPVTALLLPLALLLHAARPQVQLQQPGAELVKPGASMKLSCKASGYTFTSYWMHWVKQRPGQGLEWIGMIHPNSDNTIYYEKFKSKATLTVDKSSSTAYMQLSSLTSEDSAVYYCAIIITPVVPKFDYWGQGTTLTVSSGGGGSGGGGSGGGGSDIVMTQSHQFMSTSVGDRVSVTCKASHDVGTSVAWYQQKPGQSPKLLIYWASTRHTGVPDRFTGSGSGTDFTLTISNVQSEDLADYFCQQYSSYPLTFGAGTKLELKRASDPGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - protein, 244 aa, artificial
  MESO-scFv
QVQLQQPGAELVKPGASMKLSCKASGYTFTSYWMHWVKQRPGQGLEWIGMIHPNSDNTIYYEKFKSKATLTVDKSSSTAYMQLSSLTSEDSAVYYCAIIITPVVPKFDYWGQGTTLTVSSGGGGSGGGGSGGGGSDIVMTQSHQFMSTSVGDRVSVTCKASHDVGTSVAWYQQKPGQSPKLLIYWASTRHTGVPDRFTGSGSGTDFTLTISNVQSEDLADYFCQQYSSYPLTFGAGTKLELKRA

SEQ ID NO: 108 - protein, 925 aa, artificial
  TRAC_T01-L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 109 - protein, 925 aa, artificial
  TRAC_T01-R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 110 - protein, 925 aa, artificial
  CD52_T01-L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 111 - protein, 925 aa, artificial
  CD52_T01-R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 112 - protein, 925 aa, artificial
  B2M_T01-L4
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 113 - protein, 925 aa, artificial
  B2M_T01-R4
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 114 - protein, 21 aa, artificial
  CD8alpha signal peptide
MALPVTALLLPLALLLHAARP

SEQ ID NO: 115 - protein, 20 aa, artificial
  Alternative signal peptide
METDTLLLWVLLLWVPGSTG

SEQ ID NO: 116 - protein, 16 aa, artificial
  FcgammaRIII hinge
GLAVSTISSFFPPGYQ

SEQ ID NO: 117 - protein, 45 aa, artificial
  CD8alpha hinge
TTTPAPRPPTPAPTIASQPLSLRPEACRPAAGGAVHTRGLDFACD

SEQ ID NO: 118 - protein, 231 aa, artificial
  IgG1 hinge
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 24 aa, artificial
  CD8alpha transmembrane domain
IYIWAPLAGTCGVLLLSLVITLYC

SEQ ID NO: 120 - protein, 27 aa, artificial
  CD28 transmembrane domain
FWVLVVVGGVLACYSLLVTVAFIIFWV

SEQ ID NO: 121 - protein, 27 aa, artificial
  4-1BB transmembrane domain
IISFFLALTSTALLFLLFFLTLRFSVV

SEQ ID NO: 122 - protein, 42 aa, artificial
  4-1BB co-stimulatory domain
KRGRKKLLYIFKQPFMRPVQTTQEEDGCSCRFPEEEEGGCEL

SEQ ID NO: 123 - protein, 41 aa, artificial
  CD28 co-stimulatory domain
RSKRSRLLHSDYMNMTPRRPGPTRKHYQPYAPPRDFAAYRS

SEQ ID NO: 124 - protein, 112 aa, artificial
  CD3zeta signalling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - DNA, 49 bp, artificial
  TRAC_T01-target
ttgtcccacagatatccagaaccctgaccctgccgtgtaccagctgaga

SEQ ID NO: 126 - DNA, 49 bp, artificial
  CD52_T02-target
ttcctcctactcaccatcagcctcctggttatggtacaggtaagagcaa

SEQ ID NO: 127 - DNA, 47 bp, artificial
  B2M_T02-target
ttagctgtgctcgcgctactctctctttctggcctggaggctatcca

SEQ ID NO: 128 - DNA, 1587 bp, artificial
  MESO-CAR
atggctctgcccgtcaccgctctgctgctgccactggccctgctgctgcacgcagcaaggccacaggtgcagctgcagcagcctggcgcagagctggtgaagcctggcgccagcatgaagctgtcctgcaaggcctctggctacacattcacctcctattggatgcactgggtgaagcagcgcccaggccagggactggagtggatcggcatgatccaccccaactctgacaataccatctactatgagaagtttaagagcaaggccacactgaccgtggataagagctcctctacagcctacatgcagctgagctccctgacctccgaggactctgccgtgtactattgcgccatcatcatcacacccgtggtgcctaagttcgattattggggccagggcaccacactgaccgtgtctagcggaggaggaggaagcggaggaggaggatccggcggcggcggctctgacatcgtgatgacacagagccaccagtttatgagcacctccgtgggcgaccgggtgagcgtgacctgtaaggcctcccacgatgtgggcacctctgtggcctggtaccagcagaagccaggccagagccccaagctgctgatctattgggcctccacaaggcacaccggagtgccagaccgcttcacaggatctggaagcggcaccgacttcaccctgaccatcagcaacgtgcagtccgaggacctggccgattacttctgtcagcagtactcctcttatcctctgacatttggcgcaggaaccaagctggagctgaagagggcctctgatccaggctccggcggaggaggatcctgcccttacagcaacccatccctgtgctctggaggaggaggatcttgtccctatagcaatcctagcctgtgctccggcggaggaggcagcaccacaaccccagcaccaaggccacctacacctgcaccaaccatcgcatcccagccactgtctctgaggccagaggcatgcagacctgcagcaggcggcgccgtgcacaccagaggcctggacttcgcctgcgatatctacatctgggcacctctggcaggaacatgtggcgtgctgctgctgtccctggtcatcaccctgtattgtaagcgaggccggaagaaactgctgtatattttcaaacagccctttatgagacctgtgcagactacccaggaggaagacggctgcagctgtaggttccccgaggaagaggaaggcgggtgtgagctgagggtcaagtttagccgctccgcagatgcccctgcttaccagcaggggcagaatcagctgtataacgagctgaatctgggacggagagaggaatacgacgtgctggataaaaggcgcgggagagaccccgaaatgggaggcaagccacgacggaaaaacccccaggagggcctgtacaatgaactgcagaaggacaaaatggcagaggcctatagtgaaatcgggatgaagggagagagaaggcgcggcaaagggcacgatggcctgtaccaggggctgtctactgccaccaaggacacctatgatgctctgcatatgcaggcactgcctccaaggtga

SEQ ID NO: 129 - DNA, 1536 bp, artificial
  FAP1-41BB-CAR
atggcactgccagtgaccgcactgctgctgccactggcactgctgctgcacgcagcacggcctcaggtgcagctggtgcagagcggagcagaggtgaagaagccaggagcctccgtgaaggtgtcttgcaagacaagcagatacacattcaccgagtataccatccactgggtgaggcaggcacctggacagaggctggagtggatcggaggcatcaaccctaacaatggcatcccaaactacaatcagaagtttaagggcagggtgacaatcaccgtggacacatctgccagcaccgcctatatggagctgagctccctgcgcagcgaggataccgccgtgtactattgcgcccggagaaggatcgcctacggctatgacgagggccacgccatggattactggggccagggcacactggtgaccgtgtctagcggctccacatctggaagcggcaagccaggctccggagagggatctacaaaggacatcgtgatgacccagtctccagatagcctggccgtgtccctgggagagagggcaaccatcaattgtaagtcctctcagagcctgctgtattccagaaaccagaagaattacctggcctggtatcagcagaagcctggccagccccctaagctgctgatcttctgggcatctacaagggagagcggagtgccagacagattttccggctctggcttcggcaccgattttacactgaccatcagctccctgcaggcagaggacgtggcagtgtactattgccagcagtacttctcctatcccctgacatttggccagggcaccaaggtggagatcttcgtgcctgtgtttctgccagcaaagcctaccacaaccccagcaccaaggccaccaacacctgcaccaaccatcgcatcccagccactgtctctgaggcctgaggcatgcaggccagcagcaggaggagcagtgcacacccgcggcctggactttgcctgcgatatctacatctgggcaccactggcaggaacatgtggagtgctgctgctgtccctggtcatcaccctgtactgtaaccaccggaataagcggggcagaaagaagctgctgtatatcttcaagcagcccttcatgaggcccgtgcagacaacccaggaggaggacggctgctcttgtaggttcccagaggaggaggagggaggatgtgagctgcgggtgaagtttagcagatccgcagatgcacctgcataccagcagggacagaaccagctgtataacgagctgaatctgggaagaagggaggagtacgacgtgctggataagagaaggggaagggacccagagatgggaggcaagccaaggaggaagaaccctcaggagggactgtacaatgagctgcagaaggacaagatggccgaggcctattctgagatcggcatgaagggagagagaaggaggggcaagggacacgatggactgtaccagggcctgagcacagccaccaaggacacctatgatgccctgcacatgcaggccctgcctccaagataa

SEQ ID NO: 130 - DNA, 1548 bp, artificial
  FAP2-41BB-CAR
atggcactgccagtgaccgcactgctgctgccactggcactgctgctgcacgcagcacggcctgaggtgcagctgcagcagtctggacctgagctggtgaagccaggagcatccgtgcgcatgtcttgtaaggccagcggctacaccttcacagattactatatgaagtgggtgaagcagtccctgggcaagtctctggagtggatcggcgacatctatcccaacaatggcgagatcccttacaaccagaagtttaagggcaaggccaccctgacagccgacaagacaagctccaccgcctatatgcagctgaatagcctgacctccgaggattctgccgtgtactattgcgtgagaggctactattacggactggcaatggactactggggacagggcacaagcgtgacctccgtggtgggaggaggaggctccggaggaggaggctctggaggcggcggcagccaggcagtggtgacacaggagagcgccctgacctccccaggagagaccgtgacactgacctgtcggtctagcacaggcgccgtgaccacaagcaactacgccaattgggtgcaggagaagcctgatagactgttcacaggcctgatcggcgccaccaacaatagggcaccaggagtgccagcacgcttttccggatctctgatcggcgacaaggccgccctgacaatcaccggagcacagaccgaggatgaggccatctatttctgcgccctgtggtacagcaaccacttcatctttggctccggcacaaaggtgaccgtgctgggcggcggcggcagcggcggcggcggctccggcggcggcggcagcttcgtgcctgtgtttctgccagcaaagcctaccacaaccccagcaccaaggccaccaacacctgcaccaaccatcgcatcccagccactgtctctgaggcctgaggcatgcaggccagcagcaggaggagcagtgcacacccgcggcctggactttgcctgcgatatctacatctgggcaccactggcaggaacatgtggagtgctgctgctgtccctggtcatcaccctgtactgtaaccaccggaataagcggggcagaaagaagctgctgtatatcttcaagcagcccttcatgaggcccgtgcagacaacccaggaggaggacggctgctcttgtaggttcccagaggaggaggagggaggatgtgagctgcgggtgaagtttagcagatccgcagatgcacctgcataccagcagggacagaaccagctgtataacgagctgaatctgggaagaagggaggagtacgacgtgctggataagagaaggggaagggacccagagatgggaggcaagccaaggaggaagaaccctcaggagggactgtacaatgagctgcagaaggacaagatggccgaggcctattctgagatcggcatgaagggagagagaaggaggggcaagggacacgatggactgtaccagggcctgagcacagccaccaaggacacctatgatgccctgcacatgcaggccctgcctccaagataa

SEQ ID NO: 131 - DNA, 1563 bp, artificial
  FAP3-41BB-CAR
atggcactgccagtgaccgcactgctgctgccactggcactgctgctgcacgcagcacggcctcaggtgcagctggtgcagtctggagcagaggtgaagaagccaggcgcctctgtgaaggtgagctgtaaggcctccggctatacctttacagagaacatcatccactgggtgcgccaggcacctggacagggactggagtggatgggctggttccacccaggctctggcagcatcaagtacaatgagaagtttaaggaccgggtgaccatgacagccgatacctccacatctaccgtgtatatggagctgagctccctgagatccgaggacacagccgtgtactattgcgcaaggcacggaggaaccggaaggggagccatggattactggggccagggcacactggtgaccgtgtctagcggaggaggaggctccggaggaggaggatctggcggcggcggcagcgacatccagatgacacagtccccatcctctctgagcgcctccgtgggcgatagggtgacaatcacctgtagagcctctaagagcgtgtccacctctgcctacagctatatgcactggtaccagcagaagccaggcaaggcccccaagctgctgatctatctggcctctaacctggagagcggagtgccttcccggttcagcggctccggatctggaacagactttacactgaccatcagctccctgcagccagaggatttcgccacctactattgccagcacagcagggagctgccctacacatttggccagggcaccaagctggagatcaagaggggcggcggcggctccggcggaggaggctccggaggcggcggcagcttcgtgcctgtgtttctgccagcaaagcctaccacaaccccagcaccaaggccaccaacacctgcaccaaccatcgcatcccagccactgtctctgaggcctgaggcatgcaggccagcagcaggaggagcagtgcacacccgcggcctggactttgcctgcgatatctacatctgggcaccactggcaggaacatgtggagtgctgctgctgtccctggtcatcaccctgtactgtaaccaccggaataagcggggcagaaagaagctgctgtatatcttcaagcagcccttcatgaggcccgtgcagacaacccaggaggaggacggctgctcttgtaggttcccagaggaggaggagggaggatgtgagctgcgggtgaagtttagcagatccgcagatgcacctgcataccagcagggacagaaccagctgtataacgagctgaatctgggaagaagggaggagtacgacgtgctggataagagaaggggaagggacccagagatgggaggcaagccaaggaggaagaaccctcaggagggactgtacaatgagctgcagaaggacaagatggccgaggcctattctgagatcggcatgaagggagagagaaggaggggcaagggacacgatggactgtaccagggcctgagcacagccaccaaggacacctatgatgccctgcacatgcaggccctgcctccaagataa

SEQ ID NO: 132 - DNA, 1542 bp, artificial
  FAP4-41BB-CAR
atggcactgccagtgaccgcactgctgctgccactggcactgctgctgcacgcagcacggcctgaggtgcagctgctggagagcggcggcggcctggtgcagcccggcggcagcctgaggctgagctgcgccgccagcggcttcaccttcagcagctacgccatgagctgggtgaggcaggcccccggcaagggcctggagtgggtgagcgccatcatcggcagcggcgccagcacctactacgccgacagcgtgaagggcaggttcaccatcagcagggacaacagcaagaacaccctgtacctgcagatgaacagcctgagggccgaggacaccgccgtgtactactgcgccaagggctggttcggcggcttcaactactggggccagggcaccctggtgaccgtgagcagcggaggaggaggctccggaggaggaggatctggcggcggcggcagcggcggcggcggctccggcggaggaggctccggaggcggcggcagcgagatcgtgctgacccagagccccggcaccctgagcctgagccccggcgagagggccaccctgagctgcagggccagccagagcgtgaccagcagctacctggcctggtaccagcagaagcccggccaggcccccaggctgctgatcaacgtgggcagcaggagggccaccggcatccccgacaggttcagcggcagcggcagcggcaccgacttcaccctgaccatcagcaggctggagcccgaggacttcgccgtgtactactgccagcagggcatcatgctgccccccaccttcggccagggcaccaaggtggagatcaagttcgtgcctgtgtttctgccagcaaagcctaccacaaccccagcaccaaggccaccaacacctgcaccaaccatcgcatcccagccactgtctctgaggcctgaggcatgcaggccagcagcaggaggagcagtgcacacccgcggcctggactttgcctgcgatatctacatctgggcaccactggcaggaacatgtggagtgctgctgctgtccctggtcatcaccctgtactgtaaccaccggaataagcggggcagaaagaagctgctgtatatcttcaagcagcccttcatgaggcccgtgcagacaacccaggaggaggacggctgctcttgtaggttcccagaggaggaggagggaggatgtgagctgcgggtgaagtttagcagatccgcagatgcacctgcataccagcagggacagaaccagctgtataacgagctgaatctgggaagaagggaggagtacgacgtgctggataagagaaggggaagggacccagagatgggaggcaagccaaggaggaagaaccctcaggagggactgtacaatgagctgcagaaggacaagatggccgaggcctattctgagatcggcatgaagggagagagaaggaggggcaagggacacgatggactgtaccagggcctgagcacagccaccaaggacacctatgatgccctgcacatgcaggccctgcctccaagataa

SEQ ID NO: 133-134 - skipped (2 entries)

SEQ ID NO: 135 - protein, 447 aa, artificial
  Pembrolizumab Heavy Chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 136 - protein, 218 aa, artificial
  Pembrolizumab Light Chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 448 aa, artificial
  Ipilumab Heavy Chain
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 215 aa, artificial
  Ipilumab Light Chain
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 446 aa, artificial
  Relatlimab Heavy Chain
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 140 - protein, 214 aa, artificial
  Relatlimab Light Chain
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 925 aa, artificial
  TGFBR2_L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 142 - protein, 925 aa, artificial
  TGFBR2_R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD